(19) United States
(12) Patent Application Publication
(10) Pub. No.: US 2026/0249298 A1
(43) Pub. Date: Aug. 27, 2026

(54) IN VITRO DIAGNOSTIC SYSTEM

(71) Applicant: Sherlock Biosciences, Inc., Newton, MA (US)

(72) Inventors: Gregory A. Dale, Wayland, MA (US); Shih-Jie Lo, Burlington, MA (US); Brian James, Newton, MA (US); Eric Schneider, Catonsville, MD (US); Andrea Bowdon, Baltimore, MD (US); John A. Schanzle, Belmont, MA (US); Guy Thompson, II, Watertown, MA (US); Marco De Angeli, Barzana (IT)

(21) Appl. No.: 19/161,586

(22) PCT Filed: Mar. 29, 2024
(86) PCT No.: PCT/US24/22143

§ 371 Date: Sep. 2, 2025

Related U.S. Application Data
(60) Provisional application No. 63/456,444, filed on Mar. 31, 2023.
(60) Provisional application No. 63/469,294, filed on May 26, 2023.

(51) Int. Cl. B01L 7/00; B01L 3/00; C12Q 1/686

(52) U.S. Cl. CPC B01L 7/52; B01L 3/5029; C12Q 1/686; B01L 2200/026; B01L 2200/0668; B01L 2200/10; B01L 2200/16; B01L 2300/021; B01L 2300/046; B01L 2300/0816; B01L 2300/0864; B01L 2300/18

(57) ABSTRACT

A method and a system for identifying an attribute of a cartridge being inserted into an electronic reader includes providing identifying mark(s) on a cartridge label (or on the cartridge itself) such that one or more LEDs internal to the reader may illuminate the identifying mark(s) enabling one or more photodetectors internal to the reader to measure an optical signature from each identifying mark. The attribute of the cartridge may be indicative of one or more assays that may be performed using the cartridge, in conjunction with the reader.

### CROSS-REFERENCE TO RELATED APPLICATIONS

This application claims priority to U.S. Provisional Patent Application Nos. 63/456,444 filed Mar. 31, 2023, and 63/469,294 filed May 26, 2023, the disclosures of each of which are incorporated by reference herein in their entireties.

### BACKGROUND

The ability to rapidly diagnose diseases—particularly highly infectious diseases—is critical to preserving human health. For example, the development and widespread use of rapid, accurate COVID-19 diagnostic tests allowed infected individuals to be quickly identified and isolated, which assisted with containment of the disease. The COVID-19 pandemic has catalyzed the development and adoption of rapid testing for use in point-of-care (POC) setting or home settings around the world, and the management of many other infectious diseases may be enhanced by improved diagnostic tests.

### SUMMARY

Provided herein are diagnostic devices, systems, and methodologies useful for detecting target nucleic acid sequences. Devices, as provided herein, are able to be performed in a point-of-care (POC) setting or home setting without specialized equipment. Devices according to the present disclosure are low cost and easy to use.

In one aspect, the present embodiments are directed to a method and a system for identifying an attribute of a cartridge being inserted into an electronic reader that includes: providing identifying mark(s) on a cartridge label (or on the cartridge itself) such that one or more LEDs internal to the reader may illuminate the identifying mark(s) enabling one or more photodetectors internal to the reader to measure an optical signature from each identifying mark. The attribute of the cartridge may be indicative of one or more assays that may be performed using the cartridge, in conjunction with the reader.

In one aspect, the present embodiments are directed to a method of identifying an attribute of a cartridge configured to be inserted into an electronic reader, the method including: providing the electronic reader, the electronic reader including at least one internal LED, at least one internal photodetector, and at least one internal microprocessor communicatively coupled to both the at least one photodetector and the at least one LED; providing the cartridge, the cartridge including at least one identification mark; inserting the cartridge into the reader; illuminating, by the at least one LED, the at least one identification mark, thereby creating an illuminated identification mark; sensing, by the photodetector, an optical signature of the illuminated identification mark; and identifying, by the at least one microprocessor, at least one attribute of the cartridge based on the illuminated identification mark.

In some embodiments, illuminating the at least one identification mark occurs as the cartridge is inserted into the electronic reader.

In some embodiments, the at least one identification mark includes at least one bar code.

In some embodiments, the at least one LED includes at least a first LED and a second LED, wherein the at least one bar code includes: a first barcode used to quantify the speed at which the cartridge is inserted into the electronic reader, the first barcode being illuminated by the first LED, and a second barcode used to identify the at least one attribute of the cartridge, the second barcode being illuminated by the second LED, and wherein the at least one microprocessor uses the quantified speed to calibrate the optical signature of the second barcode, thereby allowing a proper determination of the cartridge identification to be made.

In some embodiments, the at least one internal photodetector includes at least one of a photodiode and a phototransistor.

In some embodiments, the LED includes at least one of a red LED and a blue LED.

In some embodiments, the at least one internal photodetector includes a dual mode photodetector configured to measure light emitted from at least one LED within both a first spectrum and a second spectrum, and the second spectrum does not overlap with the first spectrum.

In some embodiments, the first spectrum includes wavelengths in a range from about 600 nm to about 660 nm, and the second spectrum includes wavelengths in a range from about 730 nm to about 900 nm.

In some embodiments, the at least one identification mark includes one or more printed marks on a label disposed on a surface of the cartridge.

In some embodiments, the at least one LED includes from about 4 to about 12 identification LEDs, the one or more printed marks comprises from about 4 to about 12 printed marks, each printed mark being positioned to correspond to a position of one of the identification LEDs such that presence or lack of presence of a printed mark at a position may be sensed by the identification LED at the corresponding position.

In some embodiments, the number of identification LEDs is greater than the number of printed marks.

In some embodiments, a unique combination of marked positions on the label is associated with the attribute of the cartridge.

In some embodiments, the attribute includes a type of one or more assays that may be performed using the cartridge.

In some embodiments, the microprocessor executes one or more preloaded routines based on the attribute that is determined as a result of the method of identifying an attribute of a cartridge.

In some embodiments, the one or more printed marks includes one or more position sensing features.

In some embodiments, the one or more position sensing features includes: a first position sensing feature positioned on the label such that a centerline of the first position sensing feature is positioned so as to be slightly above a centerline of a first component of the reader when the cartridge is positioned correctly within the reader; and a second position sensing feature positioned on the label such that a centerline of the second position sensing feature is positioned so as to be slightly below a centerline of a second component of the reader when the cartridge is positioned correctly within the reader; the method including determining, by the microprocessor, that the cartridge is or is not positioned correctly within the reader based on: 1) the position of the first position sensing feature relative to the first component of the reader, and 2) the position of the second position sensing feature relative to the second component of the reader, wherein each of the first component and the second component includes at least one of the at least one internal photodetector and the at least one LED.

In some embodiments, wherein the reader is configured to identify the attribute of the cartridge based on multiple modes of operation, the multiple modes of operation including: static identification including identification of the attribute, via the at least one identification mark and the reader, after the cartridge is inserted into the reader; and dynamic identification including identification of the attribute, via the at least one identification mark and the reader, while the cartridge is being inserted into the reader.

In some embodiments, the one or more printed marks is printed with black ink on the label on top of a background that is at least one of white and gray.

In some embodiments, the cartridge includes at least one lyophilized bead disposed therein, the at least one lyophilized bead including at least one of a lyophilized lysis bead and a lyophilized polymerase chain reaction (PCR) bead.

In another aspect, the present embodiments are directed to a method including: obtaining a biological sample from a subject; incubating the biological sample with at least one of a reagent and a buffer, thereby producing a biological solution; performing a lysis step on the biological solution; passively cooling the biological solution; amplifying one or more target nucleic acid(s) in the biological solution by isothermal amplification; incubating the biological solution with a composition including: a CRISPR/Cas enzyme having collateral cleavage activity; a guide RNA that specifically hybridizes with one target nucleic acid; and a detectably labeled nucleic acid probe, wherein hybridization of the guide RNA with the target nucleic acid induces or increases collateral cleavage activity of the CRISPR/Cas enzyme and the CRISPR/Cas enzyme cleaves the detectably labelled nucleic acid probe, wherein cleavage of the detectably labelled nucleic acid probe results in an increase in detectable label; and determining the target nucleic acid is present in the biological sample based on detecting an increase in the detectable label.

In some embodiments, the lysis step includes a thermal lysis step performed at a temperature in a range from about 70 degrees C to about 95 degrees C, and wherein the thermal lysis step is performed in a first heating zone.

In some embodiments, passively cooling the biological solution includes flowing the biological solution through an internal passage of a cartridge, wherein the internal passage is vertically oriented, and wherein flowing the biological solution through the internal passage includes gravity flow.

In some embodiments, the isothermal amplification step includes amplifying the biological solution at a temperature in a range from about 50 degrees C to about 70 degrees C, and the isothermal amplification step is performed in a second heating zone.

In some embodiments, the isothermal amplification step includes loop-mediated isothermal amplification (LAMP).

In some embodiments, the detectably labeled nucleic acid probe is labeled with a fluorescent label.

In some embodiments, the fluorescent label includes a fluorescent group at the 5′ end and a quenching group at the 3′ end.

In some embodiments, determining the target nucleic acid is present in the biological sample includes: optically illuminating the biological solution; and detecting at least one fluorescent signature using at least one of a photodiode and a phototransistor, the at least one fluorescent signature being indicative of presence of at least one target nucleic acid.

In some embodiments, optically illuminating the biological solution includes optically illuminating the biological solution using a light-emitting diode (LED) to emit light at a wavelength in a range from about 430 nm to about 500 nm.

In some embodiments, detecting at least one fluorescent signature includes detecting the at least one fluorescent signature without amplifying the at least one fluorescent signature.

In some embodiments, the target nucleic acid is eukaryotic and/or prokaryotic.

In some embodiments, the target nucleic acid is protozoan, bacterial, viral, and/or fungal.

In some embodiments, the target nucleic acid is from Chlamydia trachomatis, Neisseria gonorrhoeae, influenza A, influenza B, SARS-COV-2, respiratory syncytial virus (RSV), and/or Trichomonas vaginalis.

In some embodiments, detecting at least one fluorescent signature includes passing the at least one fluorescent signature through a gel filter.

In another aspect, the present embodiments are directed to a method of detecting the presence of at least one target nucleic acid, the method including: obtaining from a subject, a biological sample via a sample container; incubating the biological sample with at least one of a reagent and a buffer via the sample container, thereby producing a biological solution; inserting the sample container into a cartridge such that the biological solution flows into an interior chamber of the cartridge, the interior chamber including a first heating zone; inserting the cartridge into an electronic reader including multiple heating elements for creating the first heating zone and a second heating zone within the cartridge; performing a lysis step on the biological solution within the first heating zone; passively cooling the biological solution by opening an internal passage of the cartridge such that the biological solution flows via gravity feed into the internal passage, the internal passage being fluidly downstream of, and vertically below, the interior chamber; amplifying one or more target nucleic acid(s) in the biological solution by isothermal amplification within the second heating zone which comprises multiple reaction chambers fluidly downstream of the internal passage; wherein each of the multiple reaction chambers includes: a CRISPR/Cas enzyme having collateral cleavage activity; a guide RNA that specifically hybridizes with one target nucleic acid; and a detectably labeled nucleic acid probe, wherein hybridization of the guide RNA with the target nucleic acid induces or increases collateral cleavage activity of the CRISPR/Cas enzyme and the CRISPR/Cas enzyme cleaves the detectably labelled nucleic acid probe, and wherein cleavage of the detectably labeled nucleic acid probe results in an increase in detectable label; illuminating the biological solution within each of the multiple reaction chambers via a plurality of optical energy sources, each energy source of the plurality of optical energy sources being disposed in the vicinity of one of the multiple reaction chambers; and determining the presence of at least one target nucleic acid within the biological solution based on presence or level of the detectable label via a detection device.

In another aspect, the present embodiments are directed to a system for performing a nucleic acid diagnostic test, including: a durable electronic device capable of accepting a consumable cartridge; and the consumable cartridge configured to be installed into the electronic device and including reagents used in the nucleic acid diagnostic test.

In some embodiments, the diagnostic test uses one or more reagents for CRISPR/Cas detection.

In some embodiments, two or more separate amplification reactions occur within the consumable cartridge.

In some embodiments, 8 amplification reactions occur within the consumable cartridge.

In some embodiments, fluorescence detection is used to measure molecular amplification.

In some embodiments, excitation is used to aid fluorescence detection.

In some embodiments, optical filtration is used to aid fluorescence detection.

In some embodiments, thermal processing of the sample is conducted within the consumable cartridge.

In some embodiments, thermal lysis of the sample is conducted within the consumable cartridge.

In some embodiments, gravity is used for fluidic motivation within the consumable cartridge.

In some embodiments, at least one result is displayed as a combination of: 1) lighted indicators on the electronic device, and 2) graphics on the consumable cartridge.

In some embodiments, the electronic device is configured to operate with multiple types of consumable cartridges.

In some embodiments, the electronic device automatically detects the configuration of the consumable cartridge.

In some embodiments, the electronic device uses optical excitation and detection to determine if a reaction chamber in the consumable cartridge contains a reagent.

In some embodiments, the determination occurs in a continuous manner.

In some embodiments, the determination occurs in less than 1 second.

In some embodiments, the electronic device uses optical excitation and detection to determine if a reaction chamber in the consumable cartridge contains a liquid.

In some embodiments, the liquid contains gas.

In some embodiments, the liquid includes a sample to be tested.

In some embodiments, the determination occurs in a continuous manner.

In some embodiments, the determination occurs in less than 1 second.

In some embodiments, the electronic device uses optical excitation and detection to determine if a reaction chamber contains a gas.

In another aspect, the present embodiments are directed to an electronic device for performing a nucleic acid diagnostic test in conjunction with a cartridge, the device including: an electronic subsystem that executes a test sequence based on preprogrammed parameters and unique parameters based on the type of cartridge installed; a mechanical subsystem that accepts and locates the cartridge; a thermal subsystem that heats two reaction zones within the cartridge; an optical subsystem that excites, filters, and detects fluorescence in real time; and a microfluidic control subsystem that actuates features on the cartridge to control fluidic flow within the cartridge.

In some embodiments, the mechanical subsystem supports each of the electronic subsystem, the thermal subsystem, the optical subsystem, and the microfluidic control subsystem.

In another aspect, the present embodiments are directed to a microfluidic cartridge for performing a nucleic acid diagnostic test in conjunction with an electronic device, the microfluidic cartridge including: an outer casing that interfaces with the electronic device; reagents contained within the casing; a fluidic valve that is actuated by the electronic device; filter elements that allow passage of air through the outer casing and retain liquids; and visual indication that communicates identifying information to the electronic device through at least one of absorbance and reflectance at predetermined locations.

In another aspect, the present embodiments are directed to a diagnostic cartridge identification method, including: providing an apparatus for identifying a cartridge, the apparatus comprising: an optical module for measuring an optical signature within a first spectrum, wherein the optical module measures separate optical targets within the first spectrum for identifying a cartridge type. In some embodiments, the optical module is configured to measure fluorescence in a second spectrum, the second spectrum being different than the first spectrum, the second spectrum for detecting the presence, within the cartridge, of at least one nucleic acid of a predetermined group of nucleic acids, the predetermined group including nucleic acids that are each associated with one or more indications.

In some embodiments, the nucleic acid includes a human sample. In some embodiments, the cartridge includes one or more reagents for CRISPR/Cas detection.

In some embodiments, the cartridge includes an identifying label comprising one or more optical targets. In some embodiments, the cartridge includes at least one printed barcode label.

In some embodiments, the method includes using a dual mode photodetector to measure both the optical signature within the first spectrum and fluorescence within the second spectrum. In some embodiments, dual mode photodetector utilizes two separate spectrums enabled by two different wavelength LEDs. In some embodiments, the LEDs are computer controlled.

In some embodiments, the at least one printed barcode is printed ink (e.g., black ink, colored ink, or combination of inks).

In some embodiments, the optical targets include fluorescent ink including specific spectral properties.

In some embodiments, the method further comprises: inserting the cartridge into the apparatus; illuminating the identifying label by one of the two LEDs; sensing the optical signature associated with the optical signature; and identifying the cartridge type based on the sensed optical signature.

In another aspect, the present embodiments are directed to a system including the reader and cartridge as provided herein.

In another aspect, the present embodiments are directed to a system for detecting the presence of a nucleic acid associated with at least one indication in a biological solution, the system including: an electronic reader for performing one or more assays and displaying the results thereof; a cartridge configured to be inserted into the reader, the cartridge including a cartridge assembly; and a sample collective vessel for collecting a biological sample and transferring it into an interior chamber of the cartridge.

In some embodiments, the system includes an internal assembly including the cartridge assembly once the cartridge is inserted into the reader, the internal assembly further including: a heating unit comprising one or more heating elements; and an optical assembly including at least one LED and at least one photodetector.

In some embodiments, the cartridge assembly includes a lysis chamber for receiving the biological sample from the sample collection vessel, and one or more reaction chambers disposed fluidly downstream from the lysis chamber.

In some embodiments, the one or more heating elements include a first heating element for maintaining the lysis chamber at a first temperature and a second heating element for maintaining the one or more reaction chambers at a second temperature.

In some embodiments, the cartridge assembly includes: at least one of a buffer and a reagent disposed within the lysis chamber; at least one lyophilized lysis bead disposed within the lysis chamber; and a lyophilized PCR bead disposed within each one of the one or more reaction chambers.

In some embodiments, each of the one or more reaction chambers includes a transparent dome.

In some embodiments, the cartridge assembly includes a polymer casing forming a back surface of the cartridge and a film layer including a front surface of the cartridge, the polymer casing and film layer sandwiching each of the buffer, reagent, at least one lyophilized lysis bead and/or lyophilized PCR bead therebetween.

In some embodiments, the film layer includes a polypropylene laminate.

In some embodiments, the at least one LED includes a first LED in optical communication with a reaction chamber of the one or more reaction chambers, and wherein the at least one LED is configured to illuminate an interior of the one or more reaction chambers.

In some embodiments, the at least one LED includes a second LED in optical communication with at least one identification marker disposed on a surface of the cartridge.

In some embodiments, the at least one photodetector is configured to measure fluorescence emitted from illuminated interior of the one or more reaction chambers.

In some embodiments, each of the at least one LED and the at least one photodetector is integrated into a printed circuit board assembly (PCBA).

In some embodiments, the heating unit is disposed adjacent a front surface of the cartridge and the PCBA is disposed adjacent a back surface of the cartridge.

In some embodiments, the heating unit is integrated into the PCBA.

In some embodiments, the one or more reaction chambers include multiple reaction chambers, and wherein a type of the lyophilized PCR bead in a first reaction chamber of the multiple reaction chambers is different from a type of lyophilized PCR bead in a second reaction chamber of the multiple reaction chambers.

In some embodiments, the one or more reaction chambers include multiple reaction chambers, and each of the multiple reaction chambers includes a different type of lyophilized PCR bead configured to be used in a different reaction.

In some embodiments, the one or more reaction chambers include multiple reaction chambers, and each of the multiple reaction chambers comprises a same type of lyophilized PCR bead configured to be used in a same reaction.

In some embodiments, light emitted from the at least one LED excites at least one nucleic acid contained within the one or more reaction chambers without passing through an optical lens.

In some embodiments, the system includes a mechanical assembly, wherein the mechanical assembly is configured to laterally move the cartridge within the reader after the cartridge is inserted into the reader, upon the closing of a lid of the reader, the mechanical assembly comprising: at least one linkage coupling the lid to an internal apparatus disposed within the reader; and a cam coupled to both the at least one linkage and the internal apparatus, the cam converting the closing movement of the lid to lateral movement of the internal apparatus.

In some embodiments, the cartridge includes at least one identification mark configured to be illuminated by the at least one LED. In some embodiments, the at least one identification mark includes at least one printed barcode. In some embodiments, the at least one identification mark includes one or more printed shapes. In some embodiments, the at least one identification mark is printed in ink (e.g., black ink, colored ink, or combination of inks). In some embodiments, the at least one identification mark is associated with the at least one indication.

In some aspects, the present disclosure provides a CRISPR-based in vitro detection system. Exemplary detection system is shown in FIG. 1. In some aspects, the present disclosure provides a sample collection. In some aspects, a sample collection includes a sterile swab and a buffer/reagent container. In some aspects, the present disclosure provides a cartridge. In some aspects, a cartridge is disposable. In some aspects, the present disclosure provides a reader. In some aspects, a reader is a powered reader. In some aspects, a powered reader is a USB-powered processing device. In some aspects, the powered reader may be battery-powered.

A diagnostic device, as provided herein, provides an accurate, easy-to-use at-home test to detect a target nucleic acid, e.g., pathogens, such as viruses or bacteria, on a device that is designed for multiplex detection. An exemplary in vitro diagnostic system is a molecular test for COVID/Flu multiplex with nasal swab sample. Another exemplary in vitro test is a molecular test for a Sexually Transmitted Infection (STI) panel including a multiplex positive and negative control result using a genital (e.g., vaginal) swab.

The present disclosures include detection devices and methods of detecting one or more target nucleic acids with distinct advantages over the currently available diagnostic testing products, including, but not limited to: accuracy of molecular testing, enhanced by CRISPR technology; sensitivity and selectivity equivalent to lab-based PCR testing; multiplex capability where a single sample can run multiple assays (e.g., 8 separate chambers available for assay and control reactions); menu expandability, with rapid assay and test kit development, two programmable heating zones (e.g., thermal lysis is performed separately from amplification reactions); a low-cost durable reader designed for home use; no need for calibration or maintenance; and low-cost operation provided by microfluidic gravity flow. Furthermore, detection devices and methods of the present disclosure are easy to use. Systems of the present disclosure run from initiation to results without user intervention during test sequences.

The present disclosures also provide cartridges. In some aspects, a cartridge is suitable for use in a detection system according to the present disclosures. In some aspects, a cartridge is configured for a reader, as provided herein. A cartridge according to the present disclosures provides a number of advantages, including but not limited to: reagent cartridge identification with very low-cost components; barcode reader can read barcodes using as few as 4 additional LED components; software and photodetector components are shared to provide dual purposes; spectral coding of label cartridge ID targets is visible to common photodetector via selective LED wavelength excitation while preserving a fluorescence measurement channel with same photodetector; and unique label ID patterns provide static and dynamic sensing modes as well as position information.

### BRIEF DESCRIPTION OF THE DRAWINGS

FIG. 1A illustrates a sample collection container of an in vitro diagnostic testing platform, according to aspects of the present embodiments.

FIG. 1B illustrates a cartridge of an in vitro diagnostic testing platform, according to aspects of the present embodiments.

FIG. 1C illustrates a reader of an in vitro diagnostic testing platform, according to aspects of the present embodiments.

FIG. 1D illustrates a sample collection container inserted into a cartridge, which is then inserted into a reader of an in vitro diagnostic testing platform, according to aspects of the present embodiments.

FIG. 1E illustrates an exemplary detection device system of an in vitro diagnostic testing platform, according to aspects of the present embodiments.

FIG. 2A illustrates a positive reader result indicator, according to aspects of the present embodiments.

FIG. 2B illustrates a negative reader result indicator, according to aspects of the present embodiments.

FIG. 3 illustrates a combined summary of the user workflow and assay workflow, according to aspects of the present embodiments.

FIG. 4A illustrates a nasal swab collection, according to aspects of the present embodiments.

FIG. 4B illustrates elution of a sample in a buffer in a sample collection container, according to aspects of the present embodiments.

FIG. 5 illustrates a sample transfer to a cartridge, according to aspects of the present embodiments.

FIG. 6 illustrates cartridge installation, according to aspects of the present embodiments.

FIG. 7 illustrates reader status indicator LEDs, according to aspects of the present embodiments.

FIG. 8A illustrates cartridge lysis heating, according to aspects of the present embodiments.

FIG. 8B illustrates lysis thermal control in sectional view, according to aspects of the present embodiments.

FIG. 9 illustrates transfer of a biological solution to reaction chambers, according to aspects of the present embodiments.

FIG. 10A illustrates reaction chamber heating in sectional view, according to aspects of the present embodiments.

FIG. 10B illustrates reaction chamber thermal control in sectional view, according to aspects of the present embodiments.

FIG. 11A illustrates exemplary LAMP curves including SARS Cov-2 Assay with Target, according to aspects of the present embodiments.

FIG. 11B illustrates exemplary LAMP curves including negative control, according to aspects of the present embodiments.

FIG. 12 illustrates an exemplary run summary template, according to aspects of the present embodiments.

FIG. 13 illustrates a reader clamp mechanism in an open position, in perspective view (left) and side view (right), according to aspects of the present embodiments.

FIG. 14 illustrates a reader clamp mechanism in a closed position, in perspective view (left) and side view (right), according to aspects of the present embodiments.

FIG. 15A illustrates an optical module including an optical printed circuit board assembly (PCBA), according to aspects of the present embodiments.

FIG. 15B illustrates an optical module including an optical printed circuit board assembly (PCBA) overlaid with a cartridge inserted, according to aspects of the present embodiments.

FIG. 16 illustrates a blue LED emission spectrum, according to aspects of the present embodiments.

FIG. 17 illustrates fluorescein emission and excitation spectra, according to aspects of the present embodiments.

FIG. 18 illustrates a transmission spectrum for a Kodak Wratten 2-12 optical filter, according to aspects of the present embodiments.

FIG. 19A illustrates an optical PCBA with photodiodes, according to aspects of the present embodiments.

FIG. 19B illustrates an optical PCBA with phototransistors, according to aspects of the present embodiments.

FIG. 20A illustrates heater temperatures profiles for the lysis chamber, according to aspects of the present embodiments.

FIG. 20B illustrates heater temperature profiles for the reaction chamber, according to aspects of the present embodiments.

FIG. 21 illustrates temperature profiles for multiple reaction chambers, according to aspects of the present embodiments.

FIG. 22 illustrates a basic system operating sequence, according to aspects of the present embodiments.

FIG. 23A illustrates system timing diagrams for the overall system, according to aspects of the present embodiments.

FIG. 23B illustrates system timing diagrams for the cartridge ID sequence, according to aspects of the present embodiments.

FIG. 24A illustrates a system timing diagram for a cartridge ID sequence in high speed mode (A), according to aspects of the present embodiments.

FIG. 24B illustrates a system timing diagram for a fill detection algorithm sequence, according to aspects of the present embodiments.

FIG. 25A illustrates a system timing diagram for LAMP fluorescence measurement timing (A), according to aspects of the present embodiments.

FIG. 25B illustrates a system timing diagram for a LAMP fluorescence measurement A/D timing detail, according to aspects of the present embodiments.

FIG. 26 illustrates details of the cartridge assembly in cross-section view, according to aspects of the present embodiments.

FIG. 27 illustrates details of the cartridge assembly in exploded perspective view, according to aspects of the present embodiments.

FIG. 28A illustrates details of the static cartridge identification features, according to aspects of the present embodiments.

FIG. 28B illustrates details of the static cartridge identification features, according to aspects of the present embodiments.

FIG. 28C illustrates details of the static cartridge identification features, according to aspects of the present embodiments.

FIG. 28D illustrates details of the static cartridge identification features, according to aspects of the present embodiments.

FIG. 29 illustrates details of a respiratory cartridge panel, according to aspects of the present embodiments.

FIG. 30 illustrates details of an STI cartridge panel, according to aspects of the present embodiments.

FIG. 31 illustrates LED circuit details, according to aspects of the present embodiments.

FIG. 32 illustrates a cartridge panel with ambient light blocking color, according to aspects of the present embodiments.

FIG. 33 illustrates an exemplary LED emission spectrum, according to aspects of the present embodiments.

FIG. 34 illustrates an exemplary LED emission spectrum, according to aspects of the present embodiments.

FIG. 35 illustrates a cross-sectional side view of the reaction chamber assembly, according to aspects of the present embodiments.

FIG. 36 illustrates an enlarged cross-sectional side view of the reaction chamber assembly, according to aspects of the present embodiments.

FIG. 37 illustrates a virus detection method, according to aspects of the present embodiments.

FIG. 38 illustrates a cartridge identification method, according to aspects of the present embodiments.

FIG. 39 illustrates a fill detection method, according to aspects of the present embodiments.

FIG. 40 illustrates a LAMP fluorescence measurement timing sequence, according to aspects of the present embodiments.

FIG. 41 A illustrates an exemplary industrial design concept, according to aspects of the present embodiments.

FIG. 41B illustrates an exemplary industrial design concept, according to aspects of the present embodiments.

FIG. 41C illustrates an exemplary industrial design concept, according to aspects of the present embodiments.

FIG. 41D illustrates an exemplary industrial design concept, according to aspects of the present embodiments.

FIG. 41E illustrates an exemplary industrial design concept, according to aspects of the present embodiments.

FIG. 41F illustrates an exemplary industrial design concept, according to aspects of the present embodiments.

FIG. 42A illustrates an alternate user workflow concept, according to aspects of the present embodiments.

FIG. 42B illustrates an alternate user workflow concept, according to aspects of the present embodiments.

FIG. 42C illustrates an alternate user workflow concept, according to aspects of the present embodiments.

FIG. 43 illustrates a cross-sectional side view of the reaction chamber assembly in an alternate configuration, according to aspects of the present embodiments.

### DEFINITIONS

Ambient temperature: As used herein, the term “ambient temperature” is the temperature of surroundings. In general, the term ambient temperature is to be understood as the temperature of any object or environment surrounding an item. Measuring an ambient temperature can be accomplished by using a thermometer or sensor. The ambient temperature of an item is dependent on the temperature of the surrounding of the item. The surroundings can have any temperature, such as a temperature below 95° C., such as below 90° C. , such as below 85° C., such as below 80° C., such as below 75° C., such as below 70° C., such as below 65° C., such as below 60° C., such as below 55° C., such as below 50° C., such as below 45° C., such as below 40° C,, such as below 35° C., such as below 30° C., such as below 25° C., such as below 24° C., such as below 23° C., such as below 22° C., such as below 21° C., such as below 20° C. Exemplary ambient temperature ranges include 5° C. to 50° C., such as 10° C. to 40° C., such as 15° C. to 35° C., such as 20° C. to 30° C., such as 20° C. to 25° C., such as 20° C. to 22° C.
Biological Sample: As used herein, the term “biological sample” typically refers to a sample obtained or derived from a biological source (e.g., a tissue or organism or cell culture) of interest, as described herein. In some embodiments, a source of interest is or comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or bronchoalveolar lavages; aspirates; scrapings; bone marrow specimens; tissue biopsy specimens; surgical specimens; other body fluids, secretions, and/or excretions; and/or cells therefrom, and/or combinations or component(s) thereof, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane may be used. Such a “processed sample” may comprise, for example, nucleic acids or proteins extracted from a sample or obtained by subjecting a primary sample to techniques such as amplification or reverse transcription of mRNA, isolation and/or purification of certain components, etc.
Cellular lysate: As used herein, the term “cellular lysate” or “cell lysate” refers to a fluid containing contents of one or more disrupted cells (i.e., cells whose membrane has been disrupted). In some embodiments, a cellular lysate includes both hydrophilic and hydrophobic cellular components. In some embodiments, a cellular lysate includes predominantly hydrophilic components; in some embodiments, a cellular lysate includes predominantly hydrophobic components. In some embodiments, a cellular lysate is a lysate of one or more cells selected from the group consisting of plant cells, microbial (e.g., bacterial or fungal) cells, animal cells (e.g., mammalian cells), human cells, and combinations thereof. In some embodiments, a cellular lysate is a lysate of one or more abnormal cells, such as cancer cells. In some embodiments, a cellular lysate is a crude lysate in that little or no purification is performed after disruption of the cells; in some embodiments, such a lysate is referred to as a “primary” lysate. In some embodiments, one or more isolation or purification steps is performed on a primary lysate; however, the term “lysate” refers to a preparation that includes multiple cellular components and not to pure preparations of any individual component.
Composition: Those skilled in the art will appreciate that the term “composition”, as used herein, may be used to refer to a discrete physical entity that comprises one or more specified components. In general, unless otherwise specified, a composition may be of any form (e.g., gas, gel, liquid, solid, etc.) or combination of forms.
Complementary: As used herein, the term “complementary” refers to the overall relatedness between polymeric molecules, e.g., between polynucleotides. In some embodiments, polynucleotides such as nucleotide sequences (e.g., primer nucleotide sequences or target nucleotide sequences) are considered to be “complementary” to one another if their sequences are at least 80%, at least 85%, at least 90%, at least 95%, at least 99%, or 100% identical. In some embodiments, polynucleotides are considered to be “complementary” to one another if their sequences are at least 80%, at least 85%, at least 90%, at least 95%, at least 99%, or 100% similar. In some embodiments, polynucleotides are considered to be “complementary” to one another if they are capable of hybridizing to each other.
Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.
Detectable entity: The term “detectable entity” as used herein refers to any element, molecule, functional group, compound, fragment or moiety that is detectable. In some embodiments, a detectable entity is provided or utilized alone. In some embodiments, a detectable entity is provided and/or utilized in association with (e.g., joined to) another agent. Examples of detectable entities include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.
Determine: Many methodologies described herein include a step of “determining”. Those of ordinary skill in the art, reading the present specification, will appreciate that such “determining” can utilize or be accomplished through use of any of a variety of techniques available to those skilled in the art, including, for example, specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information, data, and/or materials from a source. In some embodiments, determining involves comparing one or more features of a sample or entity to a comparable reference.
Diagnostic information: As used herein, “diagnostic information” or “information for use in diagnosis” is information that is useful in determining whether a patient has a disease, disorder or condition and/or in classifying a disease, disorder or condition into a phenotypic category or any category having significance with regard to prognosis of a disease, disorder or condition, or likely response to treatment (either treatment in general or any particular treatment) of a disease, disorder or condition.

Similarly, “diagnosis” refers to providing any type of diagnostic information, including, but not limited to, whether a subject is likely to have or develop a disease, disorder or condition, state, staging or characteristic of a disease, disorder or condition as manifested in the subject, information related to the nature or classification of a tumor, information related to prognosis and/or information useful in selecting an appropriate treatment. Selection of treatment may include the choice of a particular therapeutic agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc.
Gel: As used herein, the term “gel” refers to viscoelastic materials whose rheological properties distinguish them from solutions, solids, etc. In some embodiments, a composition is considered to be a gel if its storage modulus (G′) is larger than its modulus (G″). In some embodiments, a composition is considered to be a gel if there are chemical or physical cross-linked networks in solution, which is distinguished from entangled molecules in viscous solution.
In vitro: The term “in vitro” as used herein refers to events that occur in an artificial environment, e.g., in a test tube or reaction vessel, in cell culture, etc., rather than within a multi-cellular organism.
Isolated: As used herein, the term “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; or c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.
Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the systems and/or methods provided herein. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.
Pandemic strain: A “pandemic” influenza strain is one that has or has capacity to cause pandemic infection of human populations. In some embodiments, a pandemic strain has caused pandemic infection. In some embodiments, such pandemic infection involves epidemic infection across multiple territories, and particularly across territories that are separated from one another (e.g., by mountains, bodies of water, as part of distinct continents, etc.) such that infections ordinarily do not pass between them.
Prognostic information and predictive information: As used herein, the terms “prognostic information” and “predictive information” are used to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.
Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.
Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.
Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.
Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane may be used. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.
Specificity: As is known in the art, “specificity” is a measure of the ability of a particular ligand to distinguish its binding partner from other potential binding partners.
Static: As used herein the term “static” in the context of cartridge identification specifies that the cartridge ID process is performed after a cartridge is fully inserted into the reader, and/or that the cartridge ID process occurs when the cartridge is not being moved.
Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject has a disease, disorder or condition, e.g., a disease, disorder or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

### DETAILED DESCRIPTION OF CERTAIN EMBODIMENTS

In some embodiments, the present disclosure provides readers, cartridges, sample collection apparatuses, detection systems, and methods for detecting one or more target nucleic acids.

### A. Reader and Assay Workflow

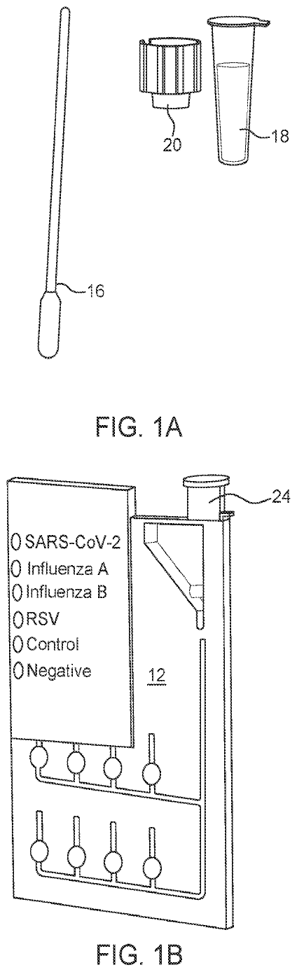

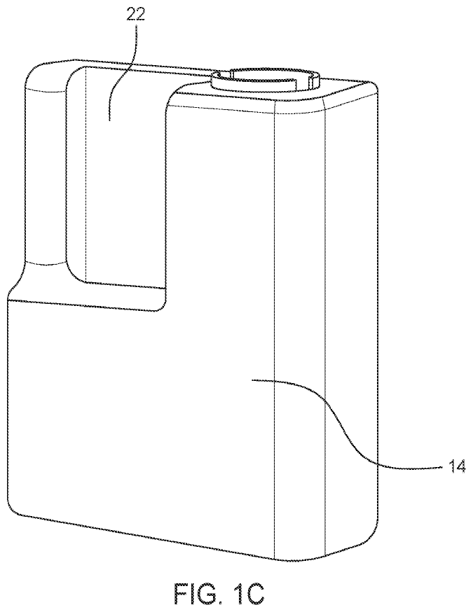

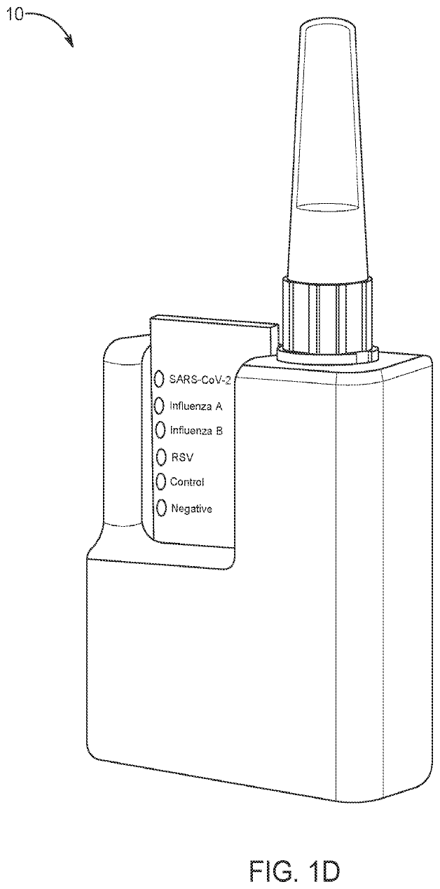

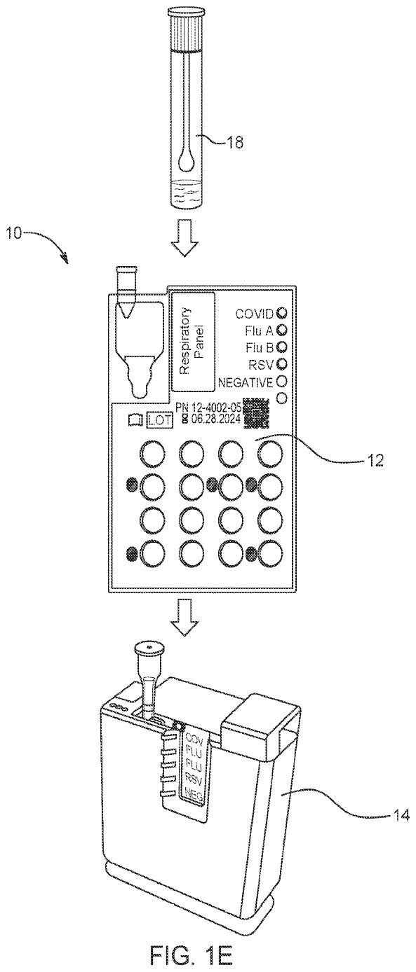

FIG. 1A illustrates a sample collection container 18, cap 20, and swab 16 of an in vitro diagnostic testing platform, according to aspects of the present embodiments. FIG. 1B illustrates a cartridge 12 of an in vitro diagnostic testing platform, according to aspects of the present embodiments. FIG. 1C illustrates a reader 14 of an in vitro diagnostic testing platform, according to aspects of the present embodiments. FIG. 1D illustrates a sample collection container inserted into a cartridge, which is then inserted into a reader of an in vitro diagnostic testing platform, according to aspects of the present embodiments. FIG. 1E illustrates an exemplary detection device system 10 of an in vitro diagnostic testing platform, including a sample collection container 18, a cartridge 12, and a reader 14, according to aspects of the present embodiments.

In some embodiments, the present disclosure provides a reader 14. In some embodiments, a reader is a powered reader 14. In some aspects, a powered reader is a USB-powered processing device. As shown in FIG. 1, a reader 14 may be part of a system 10 (shown in FIG. 1D). A reader is a device that is intended to be very user-friendly with simple workflows that a lay user (non-professional) can use properly without formal training. The system 10 may include the reader 14 (shown in FIG. 1C), a swab 16 for obtaining a biological sample (e.g., saliva, mucus, nasal swab, oral swab, etc.) sample, a fluid container/vessel/or vial 18 for receiving the sample, a cap 20 for fluidly closing the fluid container/vessel/or vial 18 (all shown n Figure IA) and a cartridge 12 shown in FIG. 1B. As shown in FIG. 1C, the reader 14 may include a slot 22 for receiving the cartridge 12. In addition, the cartridge 12 may include a receiving port 24 for interfacing with the vial 18 and/or cap 20 in order to receive the sample within the cartridge 12. FIG. 1E includes a high-level process flow illustrating the sample (e.g., saliva, mucus, nasal swab, oral swab, etc.) being introduced into the cartridge 12 (via the vial 18), and then the cartridge 12 being inserted into the reader 14.

The reader 14 automatically provides fluidic control, thermal control, and optical measurement of the cartridge 12 within a 15-to-45-minute test time. In some embodiments, a reader 14 can accept multiple types of cartridges 12, including an expanded menu after distribution to the end user allowing the end user to program the reader, as needed. In some embodiments, a reader 14 will automatically detect the cartridge 12 type and run one or more indicated test sequences for one or more assays contained therein.

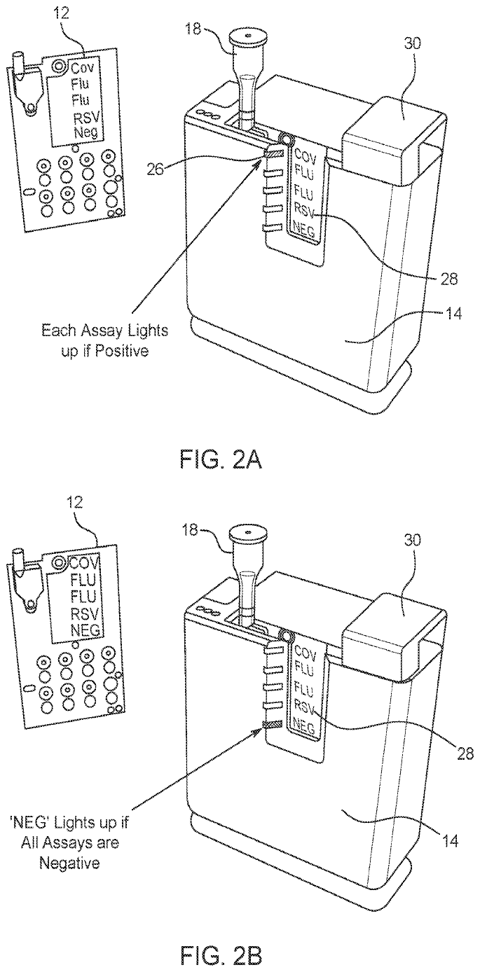

FIG. 2A illustrates a positive reader result indicator, according to aspects of the present embodiments. FIG. 2B illustrates a negative reader result indicator, according to aspects of the present embodiments. In some embodiments, results are displayed by one or more indicators 26 (i.e., lit LEDs) on the reader 14. In some embodiments, results are displayed through the combined indicators of lit LEDs on the reader 14 and corresponding labels 28 (for example, corresponding to such diagnoses as COVID-19, influenza, RSV, negative diagnoses, etc.) on the cartridge 12 (see FIGS. 2A and 2B).

In some embodiments, a reader is a durable device that performs testing processes in conjunction with the consumable elements comprising a cartridge and/or sample collection.

In some embodiments, a reader runs in an upright position. In some embodiments, an upright position allows for gravity-driven movement of fluidics (see FIG. 1C) (i.e., fluids can feed by gravity into the cartridge). In some embodiments, a reader is lightweight. In some embodiments, a reader is portable. In some embodiments, a reader may be powered by a standard USB wall charger, AC adapter, internal battery, disposable battery, and/or rechargeable battery.

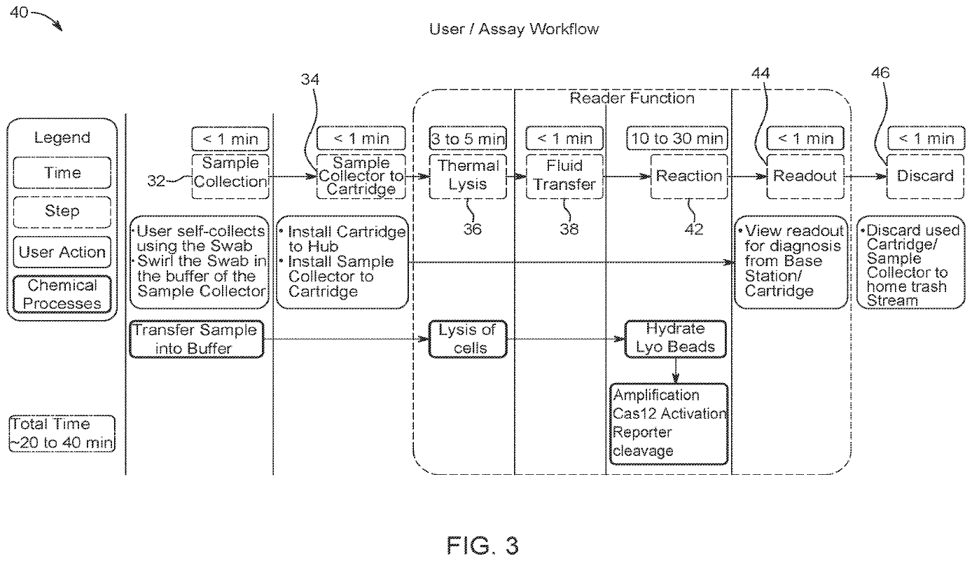

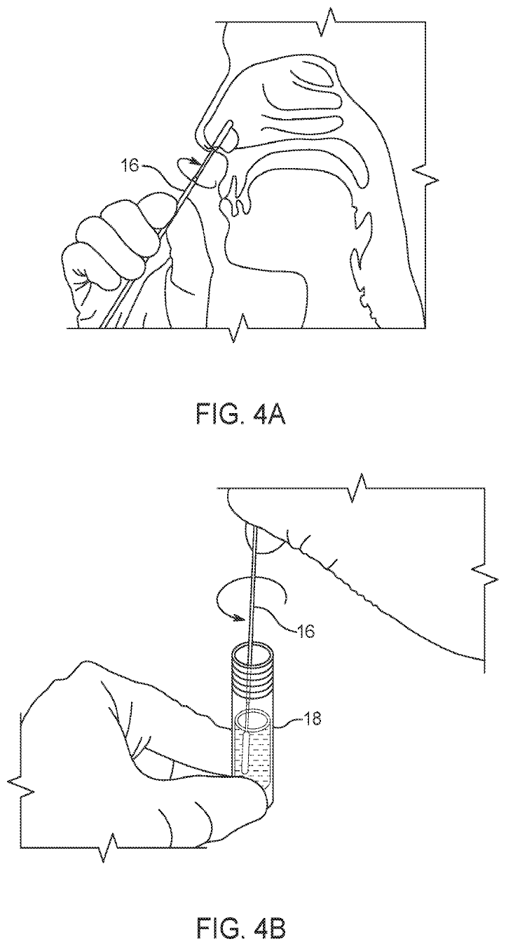

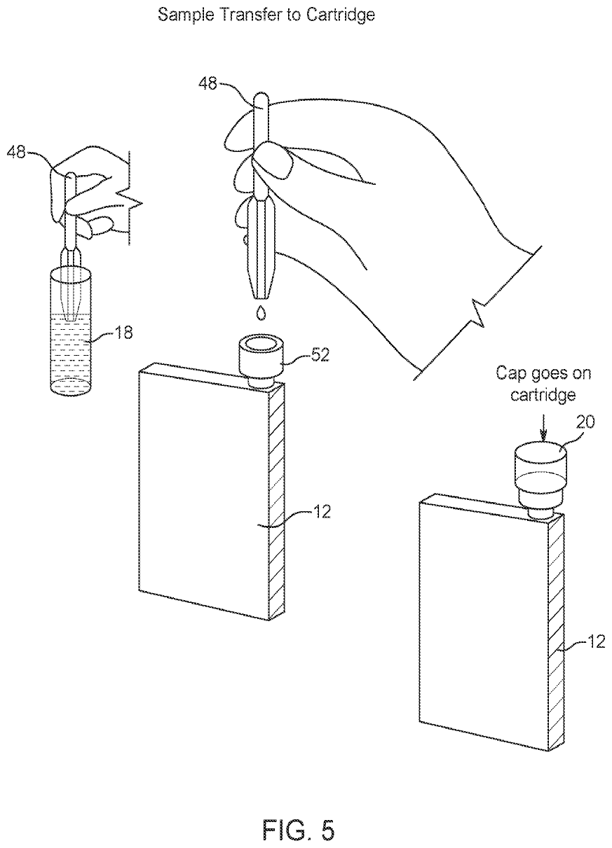

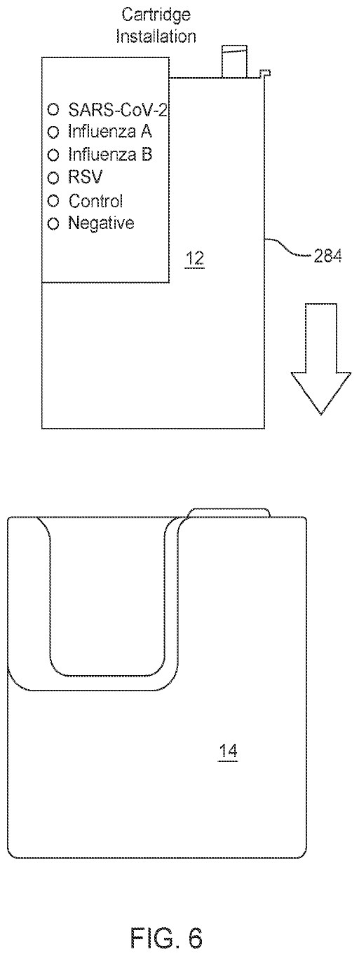

FIG. 3 illustrates a combined summary of the user workflow and assay workflow 40, according to aspects of the present embodiments. The workflow 40 illustrates the time associated with each step of the process, as well as concurrent user actions and chemical processes. For example, at step 32, the workflow 40 may include sample collection 32 for a period of about 1 minute or less during which time the user self-collects a biological sample (for example, from within one or both nostrils) a saliva or mucus or other biological sample using the swab 16 (shown in FIG. 1A), as illustrated in FIG. 4A. The swab 16 is then swirled in the sample collector (or vial) 18 which contains buffer, as shown in FIG. 4B. During the time, the sample is chemically transferred into the buffer. In some embodiments, the buffer may include a Tris buffer, for example, HC1 buffer with a pH of about 8.8, or from about 8.6 to about 9.0. At step 34, the workflow 40 may include inserting the sample collector (or vial) 18 into the cartridge 12 for a period of about a minute or less, as shown in FIGS. 1D, 1E, and 5. In the embodiment of FIGS. 1D and 1E, a cap 20 is placed on the vial 18, and then the vial is turned upside down and inserted into the cartridge 12. In the embodiments of FIG. 5, a suctioning device 48 (such as a syringe or eye dropper) may be used to transfer the sample from the sample collector (or vial) 18 into a reservoir 52 disposed at the top of the cartridge 12. The cap 20 may then be secured onto the top of the reservoir 52. During this step (34) of the workflow 40, the user installs the cartridge 12 into the slot or hub 22 of the reader 14, and also installs the sample collector (or vial) 18 into the cartridge 12 as shown in FIGS. 1E and 6, or alternately transfers the sample to the cartridge as shown in FIG. 5 and as explained above.

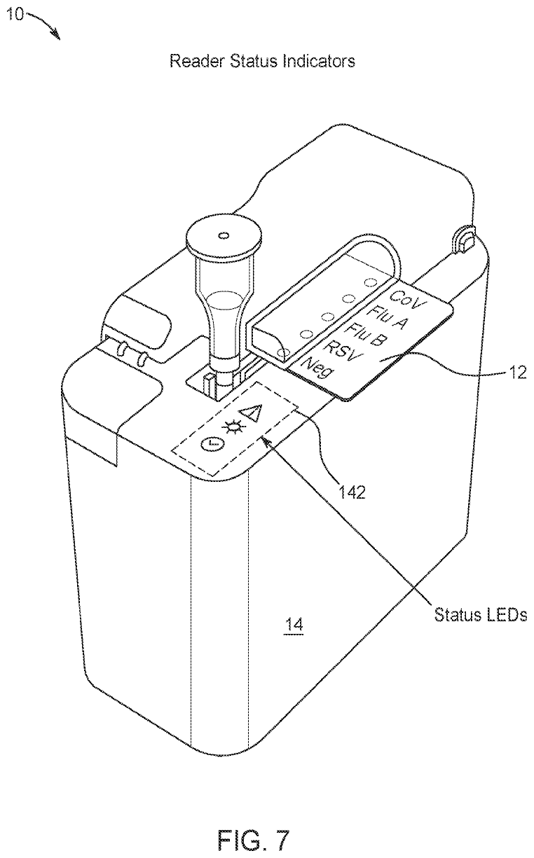

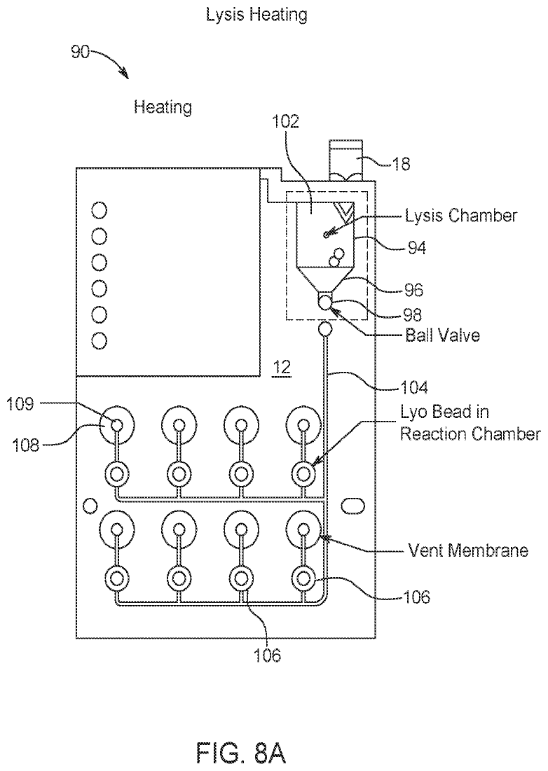

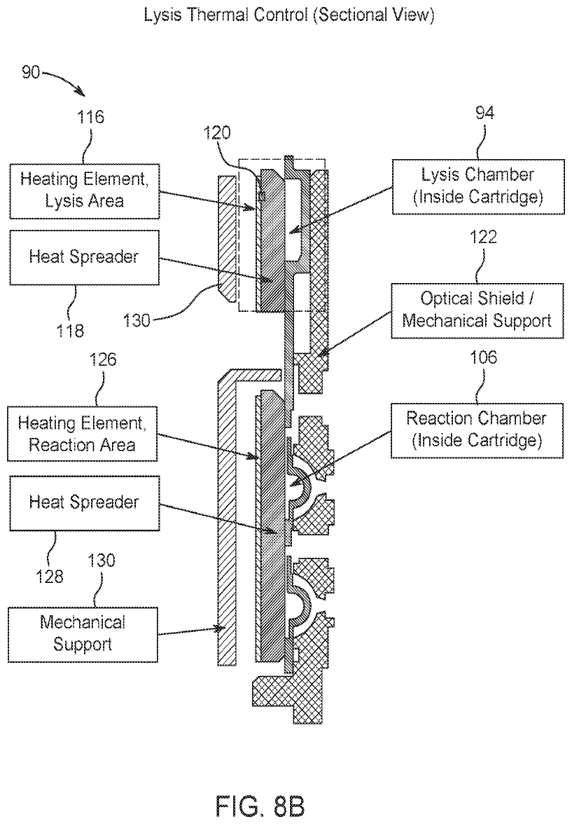

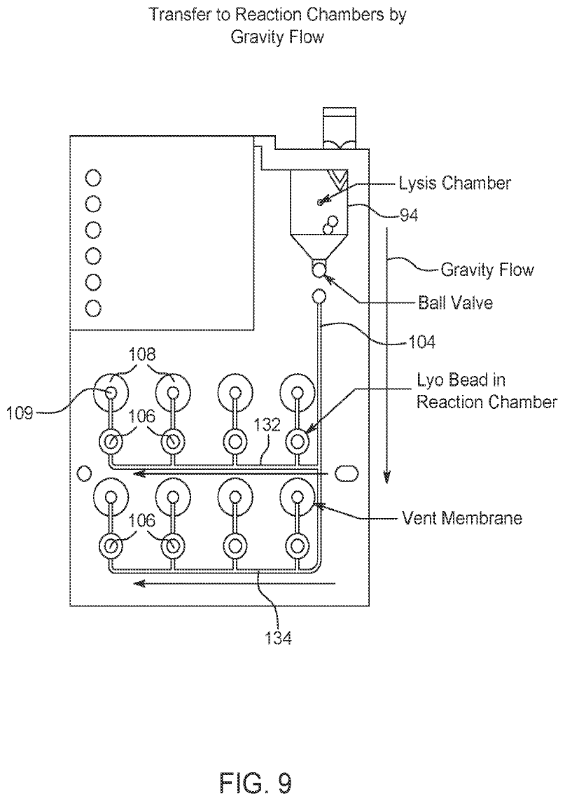

Referring still to FIG. 3, the workflow 40 may include thermal lysis at step 36, which, in some embodiments, may occur for a period of from about 3 minutes to about 5 minutes, and during which lysis of cells is performed. Thermal lysis is further described herein in conjunction with FIGS. 8A and 8B. At step 38, the workflow 40 may include fluid transfer for a period of about 1 minute or less. Fluid transfer is further described herein in conjunction with FIG. 9. At step 42, the workflow 40 may include performing a reaction for a period of about 10 to about 30 minutes. During this time, the workflow 40 may include hydration of lyophilized beads, followed up amplification, Cas-12 activation, and/or reporter cleavage. At step 44, the workflow 40 may include providing a readout of the assay results for a period of about 1 minute or less. During this time, the user views the readout diagnosis from the base station/reader 14 and cartridge, as shown in FIGS. 2A, 2B, and 7. The embodiment/system 10 of FIG. 7 may include an alternate configuration in which the cartridge 12 is inserted horizontally (i.e., rather than vertically) into the reader 14. The embodiment/system 10 of FIG. 7 may also include a panel 142 on the surface of the reader 14 that includes status LEDs. At step 46, the workflow may include discarding the used cartridge 12 and sample collector (or vial) 18 in a conventional home trash receptacle (i.e., special disposal of the cartridge 12 and sample collector (or vial) 18 are not required).

### Mechanical Subsystem

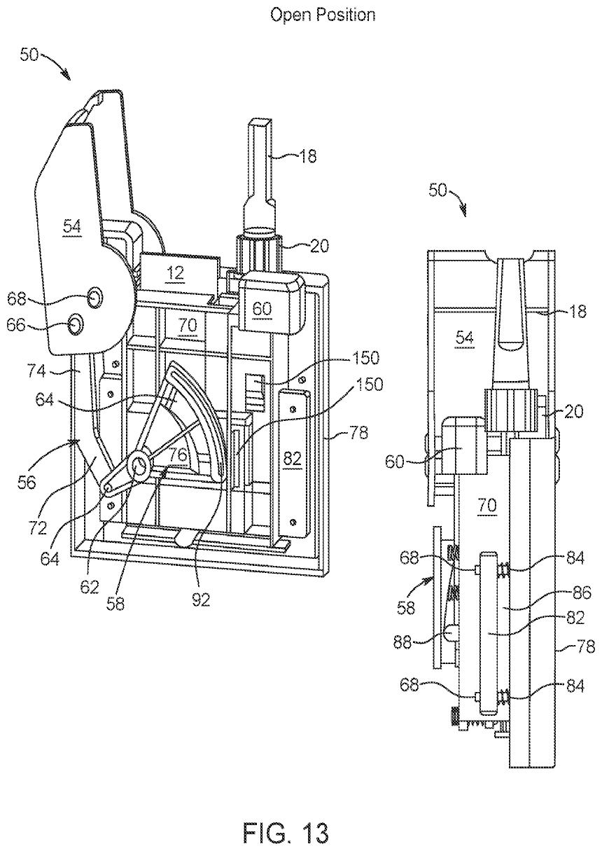

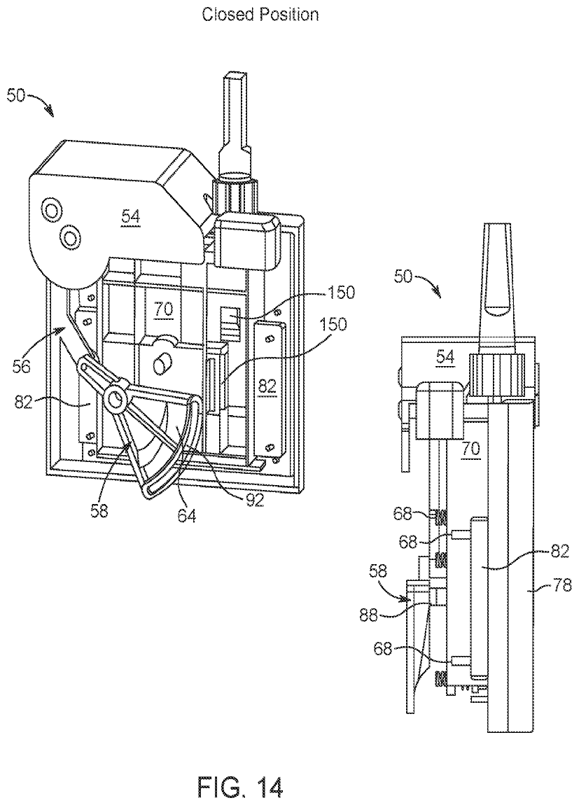

FIGS. 13 and 14 illustrate a reader clamp mechanism in an open position and a closed position, respectively, according to aspects of the present embodiments. In some embodiments, the reader 14 comprises an internal mechanical subsystem or assembly 50. In some embodiments, a mechanical subsystem comprises an internal mechanical assembly and clamping lid 54. An exemplary mechanical subsystem or assembly 50 is illustrated in FIGS. 13 and 14, which show internal components of the reader 14. The front outer casing of the reader 14 is not shown in FIGS. 13 and 14 such that the components of the mechanical sub-assembly 50 are visible. In the embodiments of FIGS. 13 and 14, the lid or clamp 54, when closed, pulls a linkage 56 up, thereby rotating a cam 58 about its axis (or first coupling) 62. The follower 88 is pushed away from the cam 58 by the rotating movement of the cam 58, in turn pushing the cartridge 12 (to the right in the side view images of FIGS. 13 and 14) via support plate 70. Accordingly, when the lid 54 is closed, it ensures the cartridge 12 is secured in the correct position within the reader 14. The cam 58 may pivot about a first coupling 62. The cam 58 may include a fan-shaped portion 76 at one end that includes a curved edge and a curved groove 64 disposed therein. The curved groove 64 has increasing or decreasing depth such that when it interfaces with the follower 88 (see side views in FIGS. 13 and 14), the support plate is translated toward and/or away from the cartridge 12 depending on whether the clamping lid 54 is being closed or opened. At an opposite end from the curved groove 64, the cam is rotatably coupled to the linkage 56 via a second coupling 64. The linkage may include a first linear member 72 and a second linear member 74 that are rigidly coupled and/or monolithic with one another, and in some embodiments, may be oriented such that they create an angle therebetween, the angle ranging from about 125 to 180 degrees, or from about 135 to 175 degrees, or from about 140 to about 170 degrees, or from about 145 to about 165 degrees, or from about 150 to 160 degrees, and/or other subranges therebetween. In some embodiments, the cam 58 may include a curved slot 92 to help support and keep the cam 58 correctly positioned as the cam 58 rotates about the first coupling 62. A tang or protrusion (not shown) extending from the support wall 70 may extend through the curved slot 92 allowing the cam 58 to rotate thereabout when the lid 54 is being opened or closed. The support wall 70 is part of an internal apparatus (i.e., internal to the reader) to help facilitate lateral movement of the cartridge within the reader. The cam 58 helps to convert the closing (i.e., rotational) movement of the reader lid to the lateral movement of the internal apparatus.

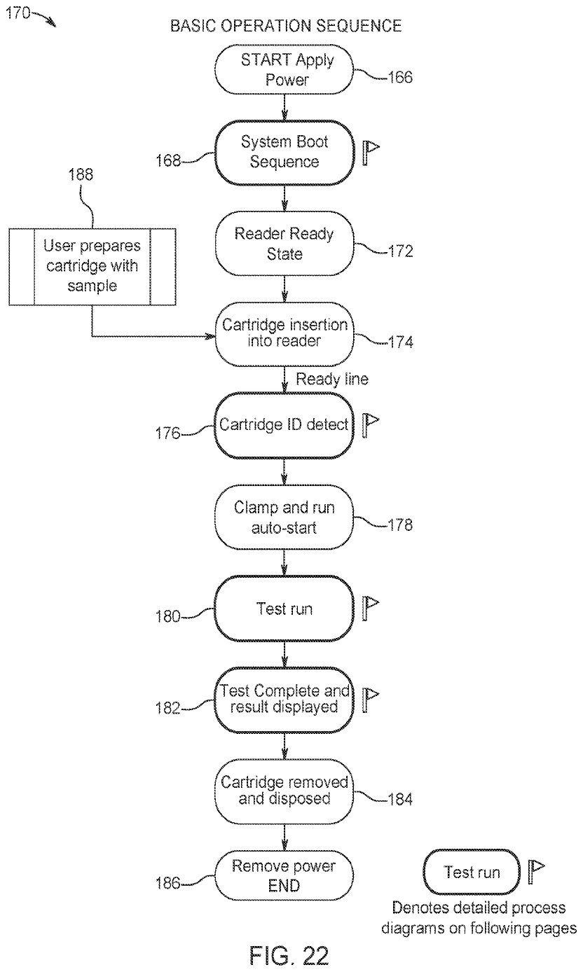

Referring still to FIGS. 13 and 14, at an opposite end from the coupling with the cam 58, the linkage 56 may be rotatably coupled to the lid 54 via a third coupling 66 disposed within the lid 54. The when the lid 54 is opened and closed, the lid 54 rotates about a fourth coupling 68 that couples the lid 54 to the body of the reader 14, and in turn, the linkage 56 and cam 58 are actuated via the third and second couplings 66, 64 respectively. The assembly 50 may include a positioning system or sensor 60 located in adjacent to and/or in the vicinity of the cap 20 and vial 18 when they are inserted into the reader 14. The positioning system 60 senses when the cartridge 12 is properly inserted into and positioned within the reader 14 such that the assay workflow may commence and/or continue, as described herein in conjunction with FIGS. 22-25. The support plate 70 may include one or more support tabs 82 on either side to help facilitate movement of the support plate 70 within the reader 14. The support tabs 82 may be rigidly coupled to the support plate 70 and may be configured with one or more through holes disposed therein such that they may slide on one or more horizontally oriented guides 68. In an open position as shown in FIG. 13, a horizontal gap 86 between the support tab 82 and edge of the outer wall 78 is shown in the side view illustration. By comparison, in the side view illustration of FIG. 14, the gap is not visible because the support plate 70 has translated to the right (toward the outer wall 78) in the closed position. In some embodiments, the assembly 50 may include one or more springs 84 disposed about the horizontal guides 68. As the lid 54 is closed, the springs 84 are compressed. When the lid 54 is opened, the springs 84 expand, thereby pushing the support plate away from the outer wall (i.e., back wall) 78 of the reader 14.

### Heating and Thermal Control

FIG. 13 illustrates a reader clamp mechanism in an open position, in perspective view (left) and side view (right), according to aspects of the present embodiments. FIG. 14 illustrates a reader clamp mechanism in a closed position, in perspective view (left) and side view (right), according to aspects of the present embodiments. In some embodiments, a cartridge 12 is installed into a reader 14 by insertion into a vertical slot 22 when a clamp (i.e., lid 54) is in an open position (FIG. 13). In some embodiments, when a clamp/lid 54 is closed to a position (FIG. 14) where it latches shut (closed position). In some embodiments, an action of closing the clamp drives a mechanical linkage 56 that translates and accurately locates the cartridge 12 to the optical module.

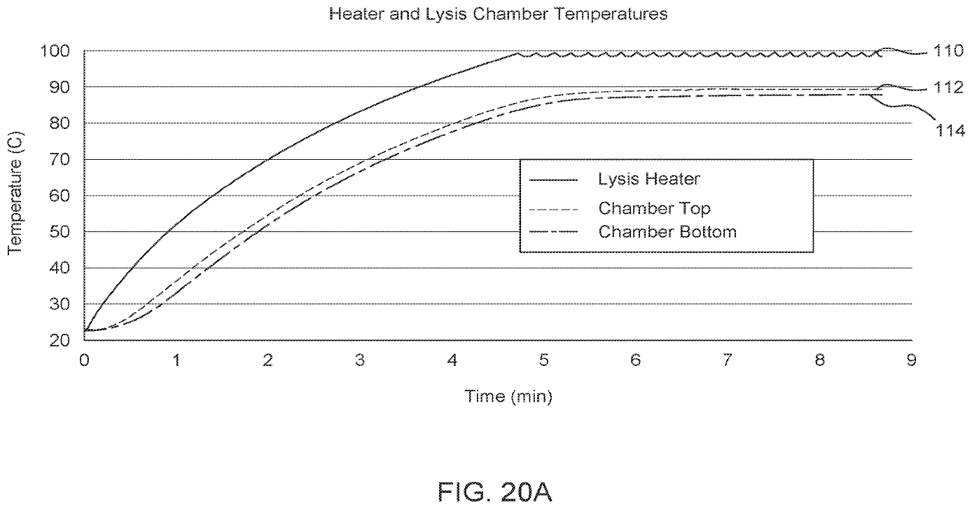

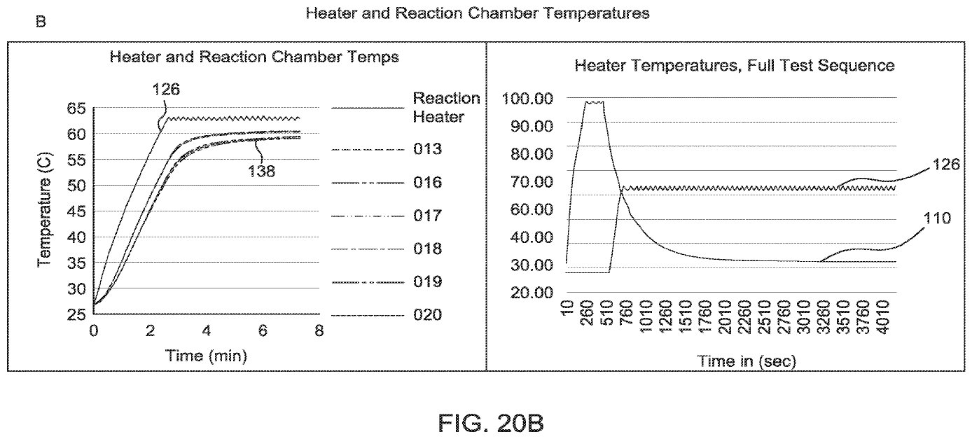

In some embodiments, a reader 14 comprises a reader thermal subsystem. In some embodiments, a reader thermal subsystem 90 heats the cartridge lysis chamber 94 and maintains accurate control using an open-loop strategy (FIGS. 8A and 20).

In some embodiments, a reader thermal subsystem 90 heats a cartridge reaction chamber 94 and maintains accurate control using an open-loop strategy (FIG. 20A). This eliminates the need for on-cartridge sensors. For example, as shown in FIG. 8B, the lysis heating element 116 includes a temperature sensor 120 that is used in a feedback loop that maintains the heater temperature at about 100 degrees C (by repeated activation and deactivation of the heater, as illustrated by the lysis temperature 110 in FIG. 20A). Heat is then transferred to via a heat spreader 118 (for example, a first heat spreader 118, see FIG. 8B) positioned between the heating element 116 and lysis chamber 94 such that the temperature at both the bottom 112 and top 114 of the lysis chamber 94 is maintained at a temperature around 90 degrees C (+/−1-2 degrees C), as shown in FIG. 20A. Accordingly, as long as the temperature 110 at the lysis heating element 116 is maintained at or around 100 degrees C, the temperature in the lysis chamber will be maintained at the target temperature of about 90 degrees C. Therefore, temperature sensors are not needed in the lysis chamber itself. Each of the first and second heating elements 116, 126 may be or include resistance heaters and/or film heaters. In some embodiments, each of the first and second heating elements 116, 126 are located on an opposite side of the cartridge 12 from the PCBA 150. In some embodiments, each of the first and second heating elements 116, 126 are integrated directly into the PCBA 150.

Referring again to FIG. 8A, the lysis chamber 94 may include a bottom tapered portion 96 that helps to funnel biological solution out of the lysis chamber 94 when a ball valve 98 is activated upon the completion of lysis heating. Biological solution may then flow through a vertical passage 104 and eventually into a plurality of reaction chambers 106, each comprising at least one lyophilized bead, and each fluidly coupled to a vent hole 109 covered by a vent membrane 108, both located in the cartridge 12 vertically above the respective reaction chamber 106 to which it is coupled. In some embodiments, the cartridge 12 may include a height from about 70 mm to about 120 mm, or from about 75 mm to about 115 mm, or from about 80 mm to about 110 mm, or from about 85 mm to about 105 mm, or from about 90 mm to about 100 mm, or about 95 mm. In some embodiments, the cartridge 12 may include a width from about 40 mm to about 90 mm, or from about 45 mm to about 85 mm, or from about 50 mm to about 80 mm, or from about 55 mm to about 75 mm, or from about 60 mm to about 70 mm, or about 75 mm. The reader thermal subsystem 90 may also include one or more mechanical supports 130 to which the heat spreader 118 (for example, a first heat spreader 118) and/or heating element 118 may be mounted.

FIG. 9 illustrates transfer of a biological solution to reaction chambers 106, according to aspects of the present embodiments. In the embodiment of FIG. 9, gravity pulls the solution through an internal passage to the 8 reaction chambers shown. A total volume of fluid may be on the order of 500 microliters, which provides enough hydrostatic head to overcome capillary action and drive the fluid to the reaction chambers. After the biological solution exits the lysis chamber 94 via the vertical passage 104, it flows into first and second horizontal passages 132, 134, each fluidly coupled to a plurality (for example, 2, 3, 4, 5, 6) of reaction chambers 106. The hydrostatic pressure created by the biological solution or fluid in the vertical passage 104 is sufficient to push any residual gas in the passages out through the vent membranes 108 and vent holes 109, while also being sufficient to push the biological fluid into each of the reaction chambers 106, via the first and second horizontal passages 132, 134. In some embodiments, the vent membranes 108 include hydrophobic vents that allow each of the reaction chambers 106 to fill (i.e., by allowing gases to exit the passages as they are filled with liquids). Each of the passages 104, 132, 134 are sized such that the overall contained fluid is of a large enough volume to create hydrostatic head, and such that the inner diameters are not so small that they create excessive resistance to flow, or surface tension, while also being small enough to avoid the formation of air bubbles. For example, in some embodiments, the inner diameters of the channels or passages 104, 132, 134 are in a range from about 400 μm to about 1200 μm, or from about 500 μm to about 1100 μm, or from about 600 μm to about 1000 μm, or from about 700 μm to about 900 μm, or from about 750 μm to about 850 μm, or about 800 μm. In some embodiments, the volume of each reaction chamber 106 is from about 30 μL to about 50 μL or from about 35 μL to about 45 μL, or about 40 μL. In some embodiments, the cartridge 12 includes about 5, 6, 7, 8, 9, or 10 reaction chambers 106. In some embodiments, the total volume of the passageways or channels 104, 132, 134 (i.e., dead volume) is from about 50 μL to about 150 μL. Accordingly, the total volume of fluid required to fill the cartridge may range from about 200 μL to about 650 μL, or from about 300 μL to about 550 μL, or from about 400 μL to about 500 μL, and/or other subranges therebetween.

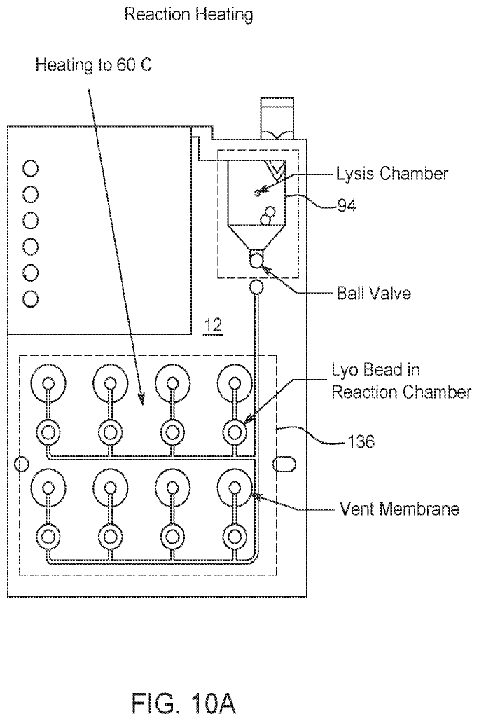

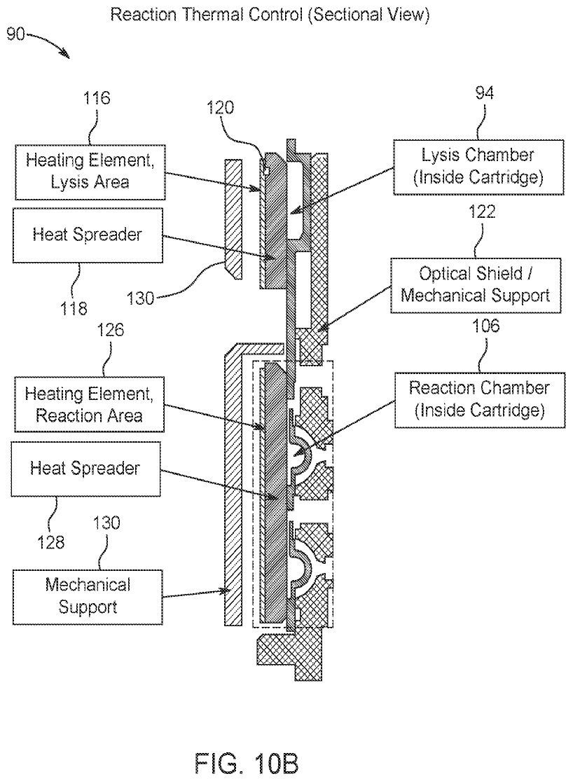

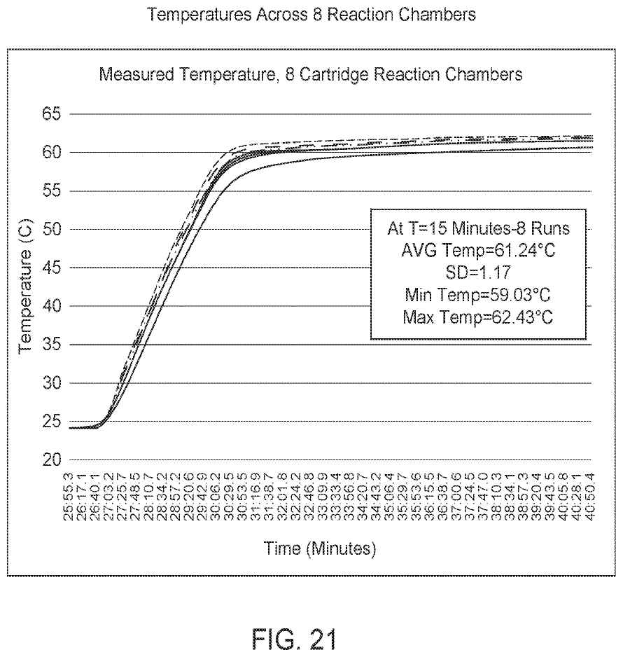

FIG. 10A illustrates reaction chamber heating in sectional view, according to aspects of the present embodiments. Referring to FIG. 10A, the cartridge 12 may include a reaction chamber heating zone 136, according to aspects of the present embodiments. While the biological fluid is flowing from the lysis chamber to the reaction chambers, as described herein, the biological fluid is passively cooled from a temperature of about 90 degrees C to about 60 degrees C. The reaction area heating element 126 (i.e., a second heating element, the lysis area heating element 116 being the first heating element) reaches and maintains a temperature of about 63 degrees C, thereby producing reaction chamber temperatures in a range from about 59 degrees C to about 60.5 degrees C, or about 60 degrees C, as shown in FIG. 20B, left side. FIG. 20B right side shows the lysis heater temperature 110 and the reaction heater temperature 126 both plotted as a function of time. The lysis heater heats up first to a temperature of about 100 degrees C, as described herein. Following lysis, the lysis heater passively cools off and the reaction heater heats up to a temperature of about 63 degrees C. FIG. 10B illustrates a side view of the reaction heater heating zone, according to aspects of the present embodiments. As shown in FIG. 21, each of the temperatures of the eight reaction chambers 106 is maintained at a temperature within a range from about 59.0 deg C to about 62.5 degrees C with an average reaction chamber 106 temperature being about 61.25 degrees C. These reaction chamber 106 temperatures (i.e., in the 59 to 62.5 degrees C range) are achieved by maintaining the reaction chamber heater/heating element 126 in a temperature range from about 63-64 degrees C (again, in an open loop configuration with the reaction chambers 106 themselves).

In some embodiments, reaction temperatures are uniform and repeatable across all cartridge chambers (e.g., eight chambers) (FIG. 20B, left side and FIG. 21).

In some embodiments, a reader 14 comprises a heater (for example, a lysis heater or heating element 116 and a second heater that includes a reaction area heater or heating element 126). In some embodiments, a reader 14 comprises a film heater. In some embodiments, a reader 14 can heat an area of a cartridge. In some embodiments, a reader comprises a film heater to heat an area of a cartridge. In some embodiments, a reader is capable of heating a cartridge lysis chamber, as described herein, to about 80° C. to about 100° C., such as about 85° C. to about 95° C. In some embodiments, a contact pressure between a heating element 116, 126, a heat spreader 118, 128, and/or an area to be heated 94, 106 is supplied by the mechanical clamping mechanism upon installation (see FIGS. 13 and 14). In some embodiments, the heat spreaders are composed of aluminum and/or other conductive materials, and comprise a thickness of from about 0.08 inches to about 0.2 inches.

In some embodiments, a reader 14 is capable of heating a cartridge reaction chamber area 106 to about 50° C. to about 70° C., such as about 60° C. (or from about 58° C. to about 63° C., or from about 58° C. to about 62° C., or from about 59° C. to about 62° C., or from about 59° C. to about 61° C., and/or to other sub-ranges between about 57° C. and about 64° C.) for the duration of a reaction (e.g., for the duration of an amplification, or for the duration of another reaction). In some embodiments, a cartridge reaction chamber 106 area of a cartridge 14 is heated to a target temperature of about 60° C. by a compliant heater plate or heat spreader 128 in a reader 14. In some embodiments, a contact pressure is supplied by the mechanical clamping mechanism upon installation, as described herein.

### Optical Module

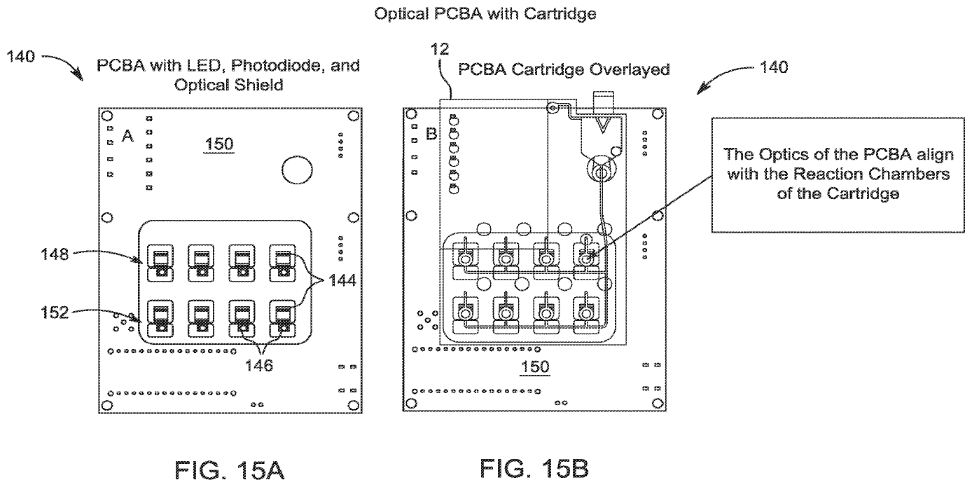

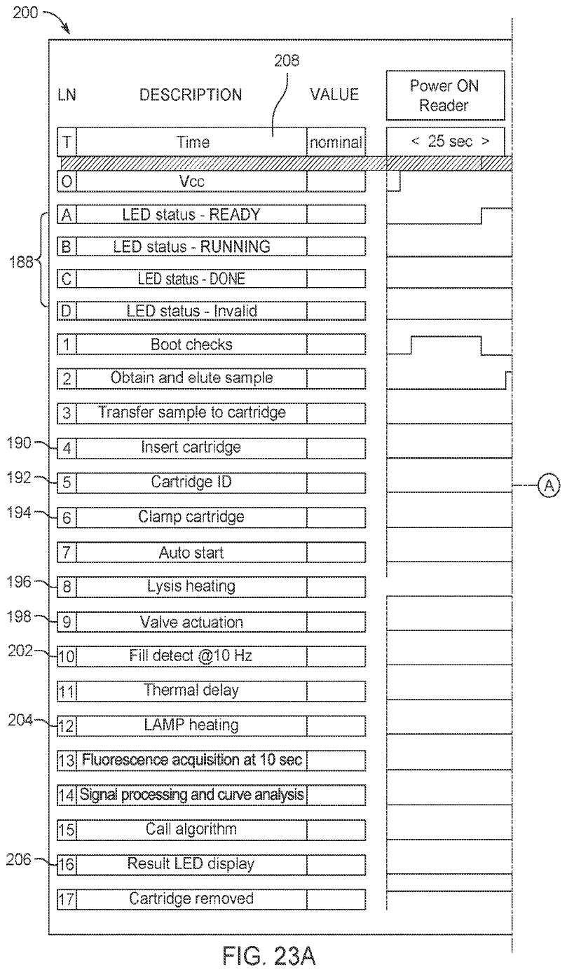

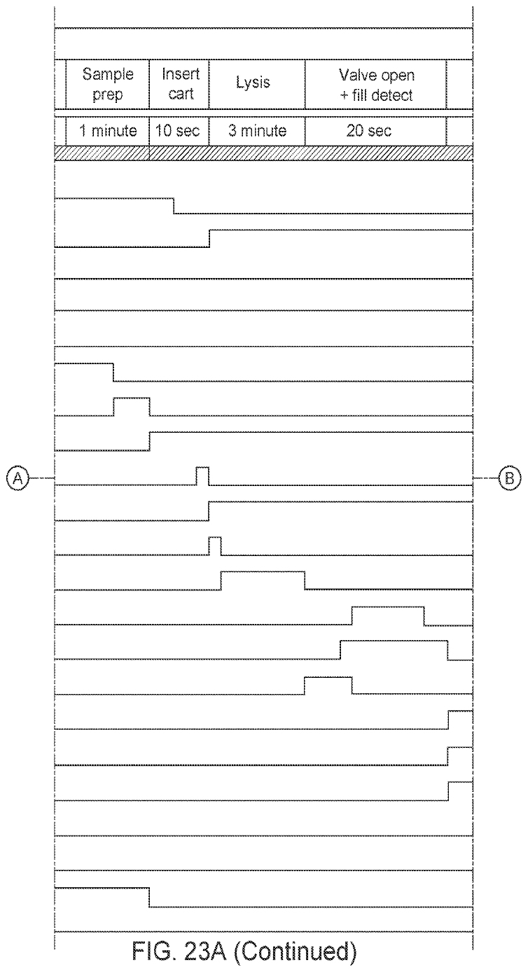

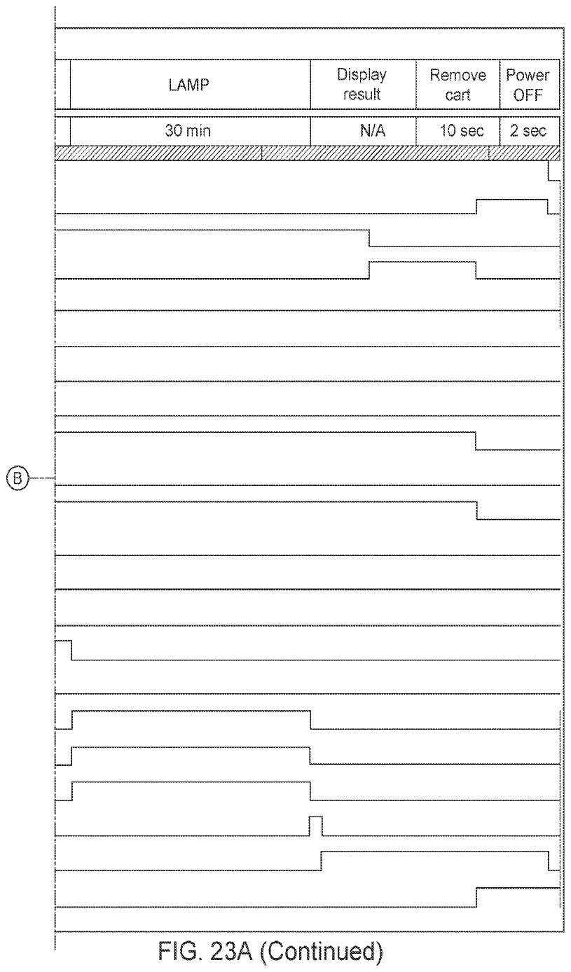

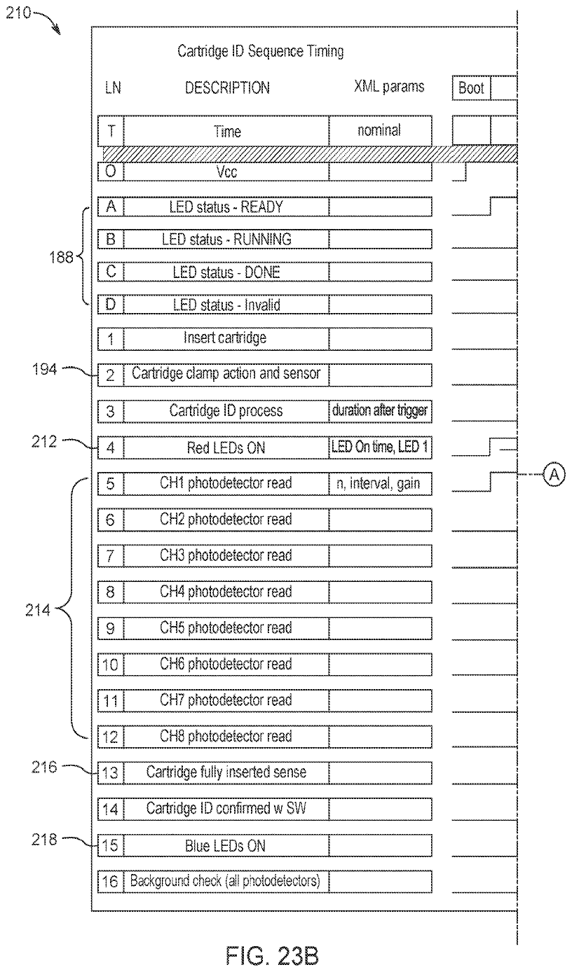

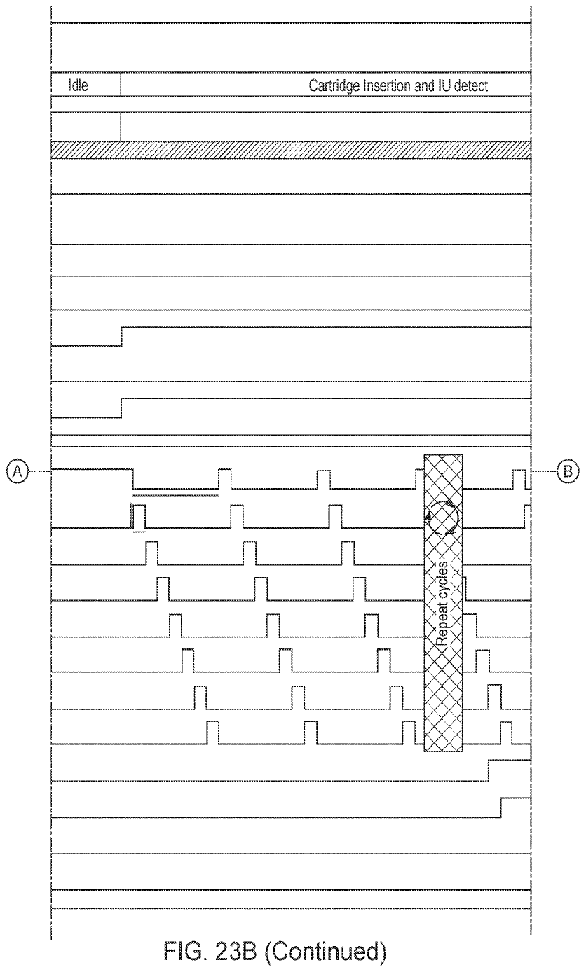

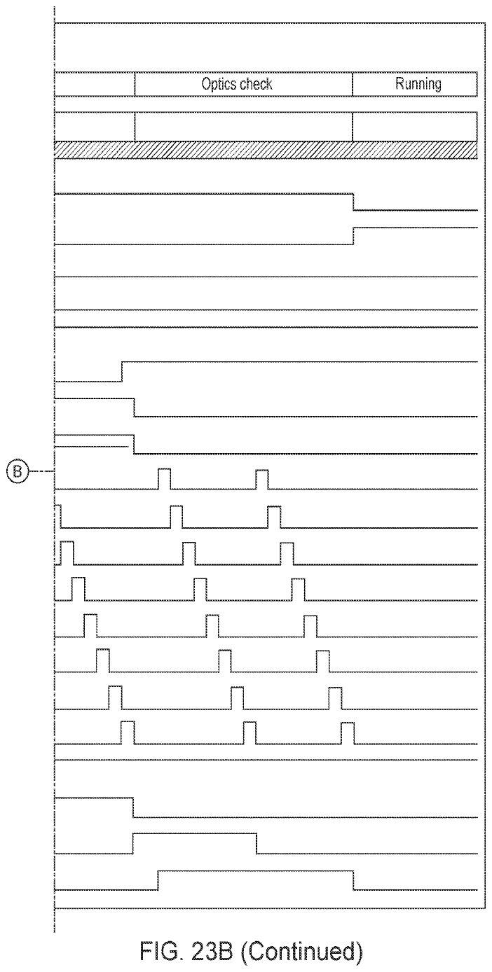

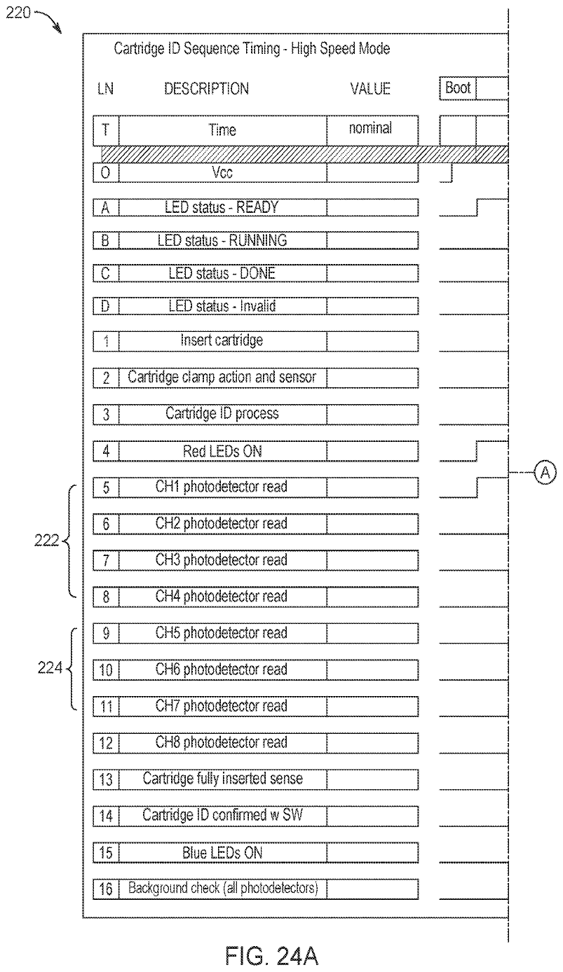

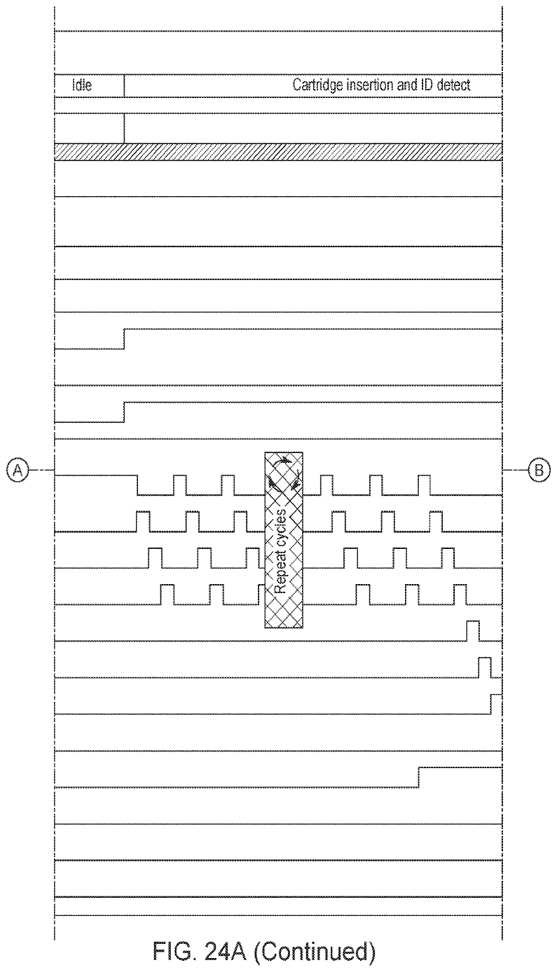

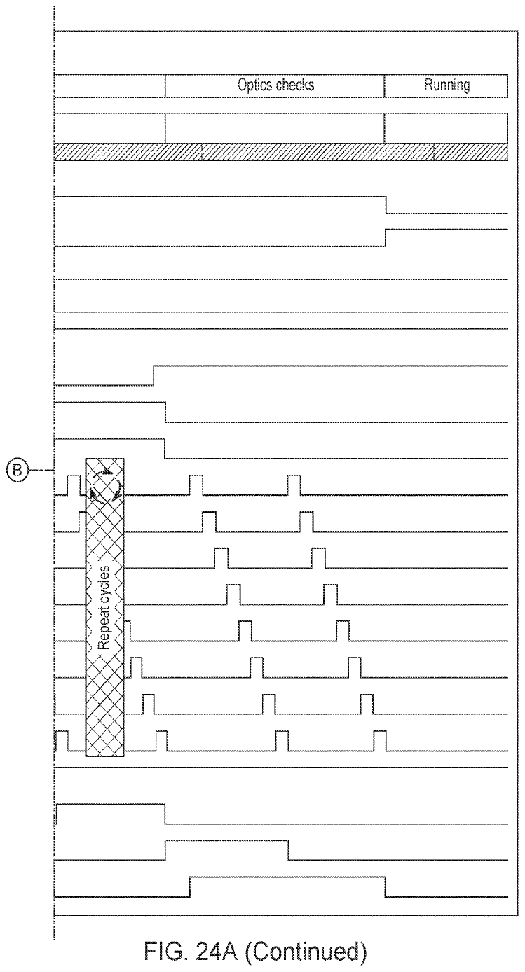

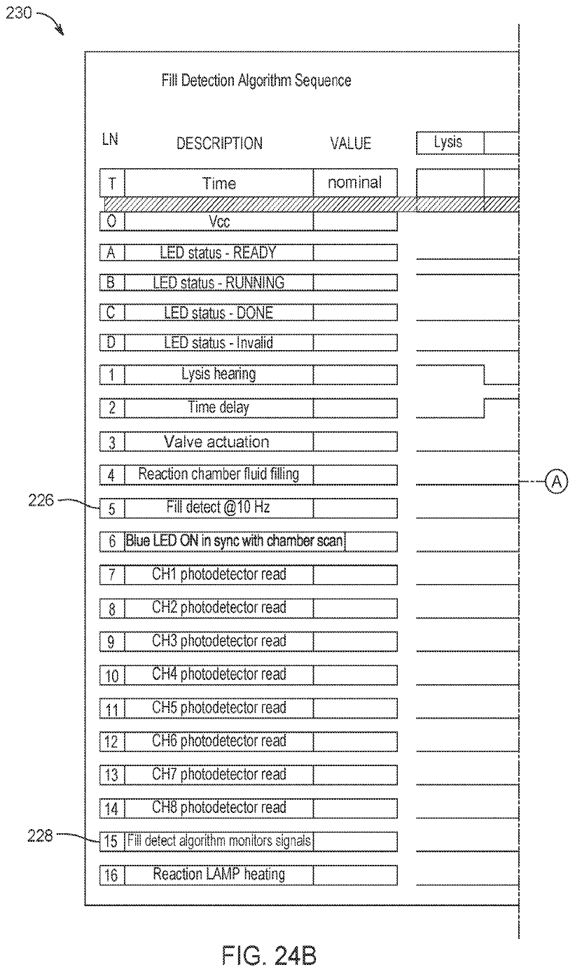

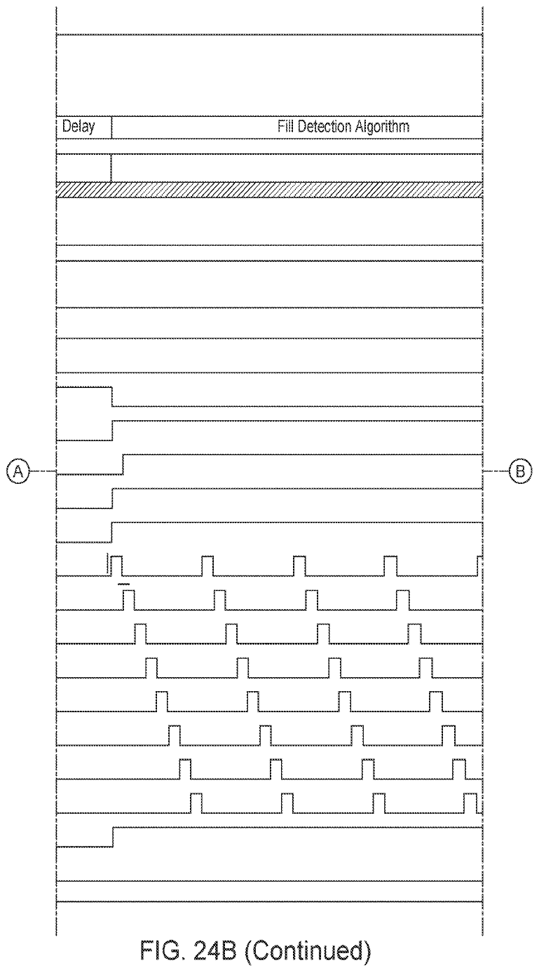

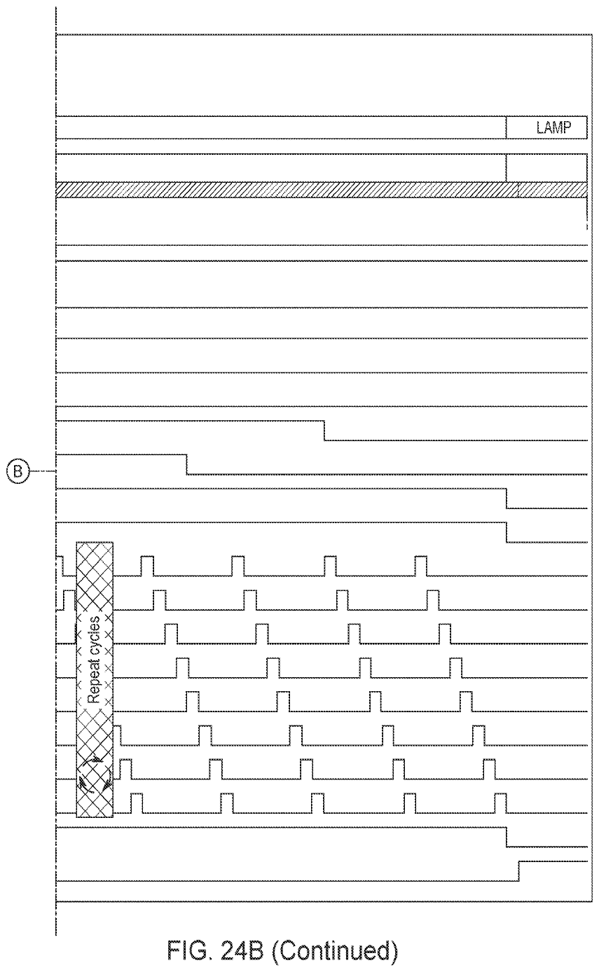

FIG. 15A illustrates an optical module 140 including an optical printed circuit board assembly (PCBA), according to aspects of the present embodiments. FIG. 1B illustrates the same embodiment of FIG. 15A with a cartridge overlaid, according to aspects of the present embodiments. In some embodiments, a reader 14 comprises an optical module 140. An optical module 140 can measure fluorescence. When a cartridge 12 is inserted, the optics module 140 is not yet required in the operating sequence (FIG. 23A and B, and FIG. 24A). In some embodiments, an optical module 140 comprises one or more photodetectors. Photodetectors of the optics module can be used for other tasks such as identifying the cartridge 12 when it is inserted into the reader 14, as discussed in further detail below.

In some embodiments, an optical module is tuned for a specific dye (e.g., a specific excitation and/or emission). In some embodiments, an optical module is tuned for the FAM dye (460 nm excitation, approximately 540 nm emission, or in some embodiments with an absorption/excitation wavelength of 495 nm and an emission wavelength of about 517 nm, and/or in some embodiments with an absorption/excitation wavelength of in a range from about 450 nm and an emission wavelength in a range from about 500 nm to about 550 nm). In some embodiments, the FAM dye comprises a carboxy fluorescein molecule that includes a carboxyl group.

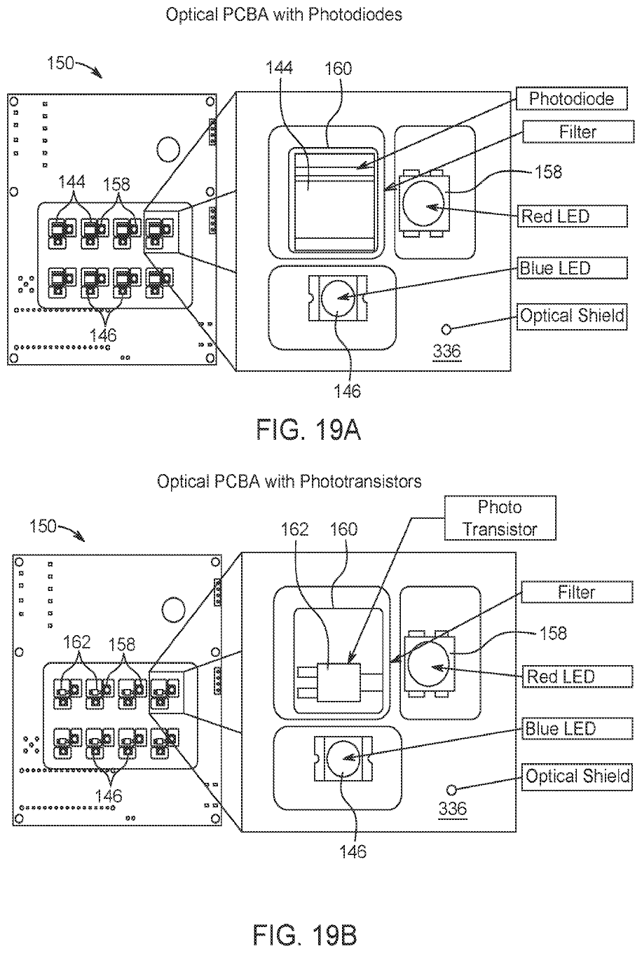

In some embodiments, an optical module 140 comprises a filter. In some embodiments, a filter is a gel filter 160 (shown in FIGS. 19A and 19B). In some embodiments, a filter is a 520 nm long-pass filter. In some embodiments, the gel filter is composed of and/or comprises polyester and/or cellulose acetate and may be adhered to the photodetector 144, 162 via an optical adhesive. The gel filter may include a thickness of from about 0.004 inches to about 0.008 inches (i.e., 4-8 mils).

During insertion of the cartridge 12, if an addition LED excitation wavelength is used that is beyond the 520 nm long pass filter, the photodetectors can be utilized to measure cartridge label ID intensity reflections. It is these reflections that can be uniquely printed in a variety of patterns to provide an identification (ID) feature (also referred to herein as barcode) on a label.

In some embodiments, a label can be as few as 2, 4, 6, 8, 10 and/or other numbers of unique ID's or as many as 256 or more. The operation of the label reading with the optical module 140 can be via a static (when inserted) or dynamic (while inserting) mode. The label color and print materials can vary from simple white label with black ink to fluorescently tagged ink to other color combinations. The label can be affixed to the cartridge 12 via adhesive, or the optical targets may be printed directly or injection molded into the cartridge plastic.

In some embodiments, an optical module 140 uses simple, low-cost components. The optics components align with the reaction chambers of the cartridge upon installation (see FIG. 15B). For example, as shown in FIG. 15A, the optical module 140 may include a first row 148 of optical components and a second row 152 of optical components. For example, each of the first and second rows 148, 152 may include four photodetectors (for example, 4 photodiodes 144) and 4 LEDs 146, for a total of 8 photodiodes 144 and 8 LEDs 146. Each of the 8 LEDs 146 may be spaced to relative to each other to match the spacing of the reaction chambers 106 shown in FIGS. 8A, 9, 10A, and 27 such that the LEDs 146 may be used for illuminating and exciting the reaction chambers 106. Excitation light from an LED on the PCBA reaches a corresponding reaction chamber, and fluorescence from a sample in the reaction chamber travels to the photodetector 144. As shown in FIGS. 13 and 14, the PCBA (i.e., printed circuit board) 150 is visible through the support wall 70 within the reader 14. In FIG. 15B, the cartridge 12 is shown overlaid on top of the PCBA 150 illustrating the reaction chambers 106 of the cartridge 12 lining up with the LEDs 146 and photodetectors 144 of the optical module 140. In the view shown in FIG. 15B, the heat spreaders 118, 128, and heating elements 116, 126 would be placed in front of the cartridge 12 (i.e., coming out of the page). Stated otherwise, once the cartridge 12 is installed in the reader 14 and positioned in place according to the description for FIGS. 13 and 14 above, the cartridge 12 is sandwiched between the heat spreaders 118, 128 and the PCBA 150. Accordingly, when the cartridge 12 is installed in the reader 14, the PCBA assembly will be adjacent to the cartridge 12 on a first side such that the LEDs 146 may excite the reaction chambers 106 and the photodetectors 144 may detect the fluorescence emitted therefrom, while at the same time, the heat spreaders 118, 128 and heating elements 116, 126 will be adjacent the cartridge 12 on a second side of the cartridge (the second side of the cartridge being opposite the first side of the cartridge) such that the heat spreaders 118, 128 and heating elements 116, 126 may heat both the lysis chamber 94 and reaction chambers 106.

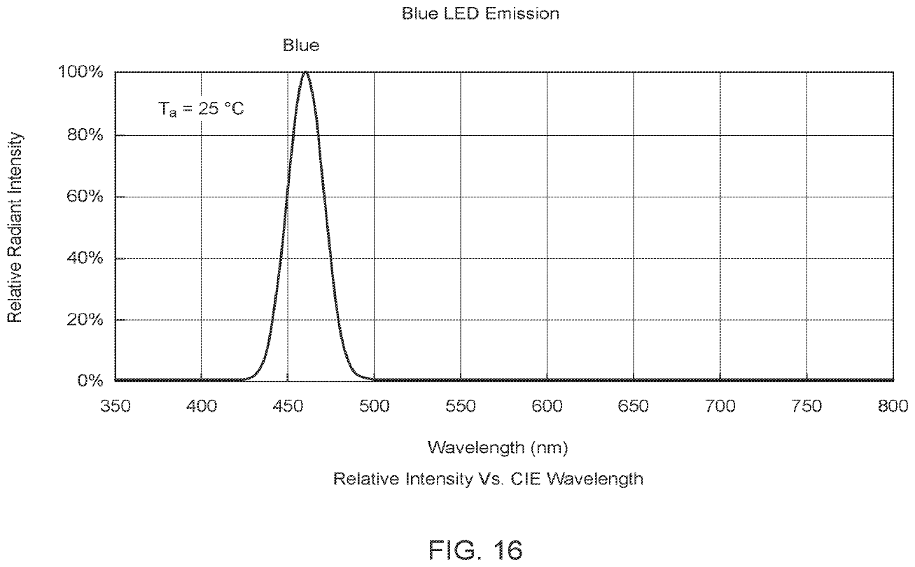

In some embodiments, during amplification reaction, the reaction chambers 106 of the cartridge 12 are excited by blue LEDs 156 (centered at about 465 nm wavelength, or for example, in a range from about 430 nm to about 490 nm) as shown in FIG. 16.

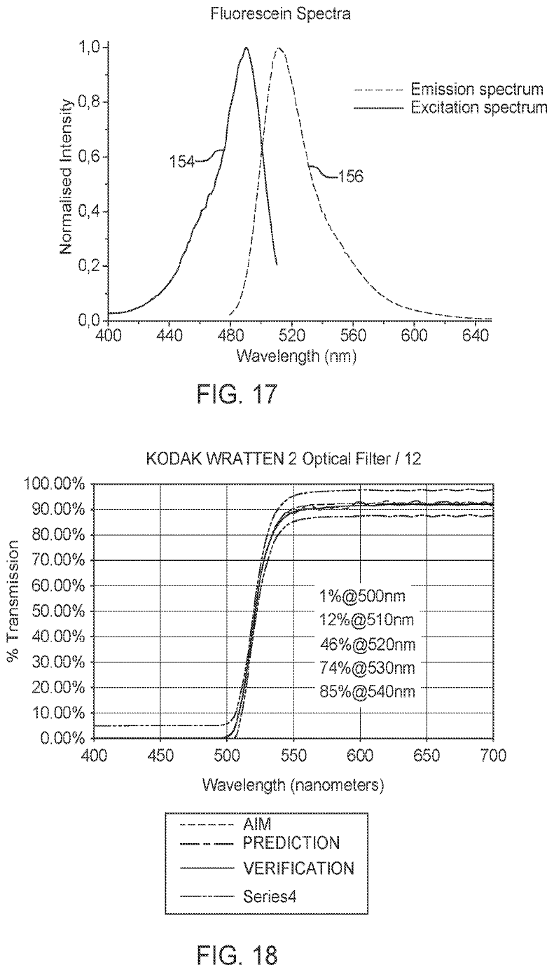

FIG. 17 illustrates fluorescein emission and excitation spectra, according to aspects of the present embodiments. In some embodiments, a positive reaction (amplification) in a reaction chamber 106 produces a logistic-type increase in fluorescent response to the excitation using a fluorescent dye (i.e., Fluorescein) (FIG. 17). For example, as shown in FIG. 17, an excitation spectrum 154 may range from about 440 nm to about 520 nm and may be centered at about 490 nm, while an emission spectrum 156 may range from about 480 nm to about 560 nm and may be centered at about 515 nm.

FIG. 18 illustrates a transmission spectrum for a Kodak Wratten 2-12 optical filter, according to aspects of the present embodiments. In some embodiments, a reflecting fluorescent emission from the reaction chambers are filtered through a low-pass filter 160 (i.e., Kodak Wratten 2-12 gel filter) (FIG. 18). As shown in FIG. 18, the transmission is only about 1% at wavelengths of 500 nm and lower, about 12% at a wavelength of 510 nm, about 46% at 520 nm, about 74% at 530 nm, about 85% at 540 nm, and more than 90% (for example, about 95%) at wavelengths of 550 nm and higher.

Therefore, according to aspects of the present embodiments, the low-pass filter may be configured such that substantially all of the light in the excitation spectrum 154 is filtered out, while substantially all of the light in the emission spectrum 156 is able to be detected. The filter 160 is shown in FIGS. 19A and 19B.

FIG. 19A illustrates an optical PCBA with photodiodes, according to aspects of the present embodiments. FIG. 19B illustrates an optical PCBA with phototransistors, according to aspects of the present embodiments. In some embodiments, two optical architectures have been successfully realized-one using photodiodes, and one using phototransistors (FIG. 19A and B). In some embodiments, red LEDs 158 are used for cartridge identification. For example, as shown in FIG. 19A, in some embodiments, the PCBA 150 may include the photodiodes 144, the first set of LEDs 146 (i.e., blue LEDs) and a second set of LEDs 158 (i.e., red LEDs). In some embodiments, the blue LEDs 146 are used for excitation of the reaction chambers 106 while the red LEDs 158 are used for identifying the cartridge 12, as further described below. In some embodiments, the red LEDs 158 are positioned adjacent the photodetectors 144, 162 about 90 degrees from the position of the blue LEDs 146 relative to the photodetectors 144, 162. Stated otherwise, the red and blue LEDs 158, 146 may be spaced about 90 degrees apart, relative to the photodetectors 144, 162. The PCBA 150 may also include the filter 160 (i.e., low-pass filter 160) and an optical shield 336. In some embodiments, the positions of the blue LEDs 146 and the red LEDs 158 are reversed (and positions of other features such as identifying markers may accordingly be adjusted as well to match the positions of the red LEDs 158, for example). In the embodiment in FIG. 19B, the PCBA 150 includes a phototransistor 162 in place of the photodiode 144 of FIG. 19A. Accordingly, the PCBA 150 may include a plurality of photodetectors that include photodiodes 144 and/or photo transistors 162. In some embodiments, a lateral spacing (center-to-center) between each of the blue and red LEDs 146, 158 and the photodetectors 144, 162 may be about 3 mm to about 9 mm, or from about 4 mm to about 8 mm, or from about 5 mm to about 7 mm, or about 5 mm, or about 6 mm, or from about 5 mm to about 6 mm.

### Software/Firmware

In some embodiments, a reader comprises software and/or firmware. The system software requirements are defined as a subsystem in an SRS (Software Requirements Specification).

### Overall Sequence Control

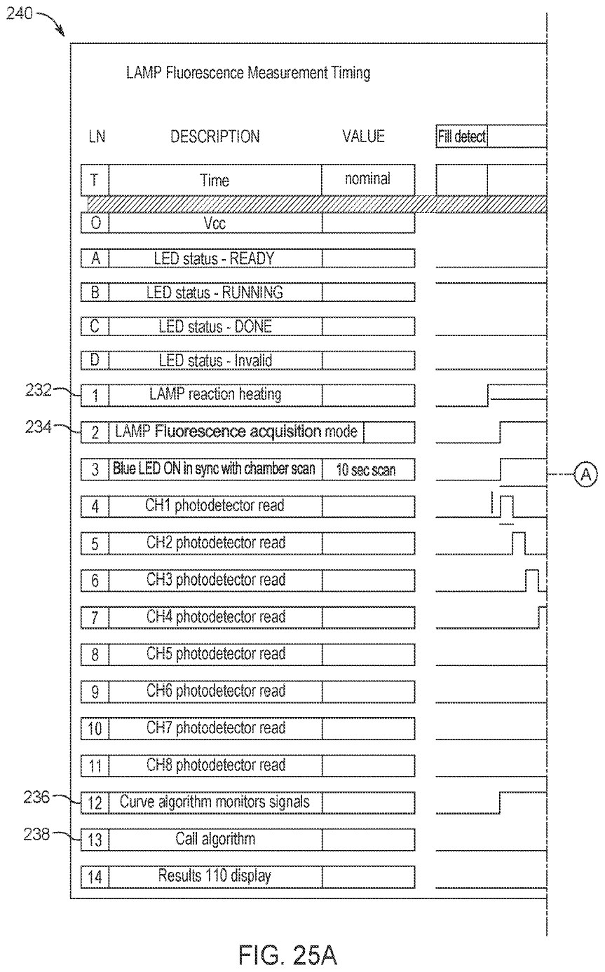

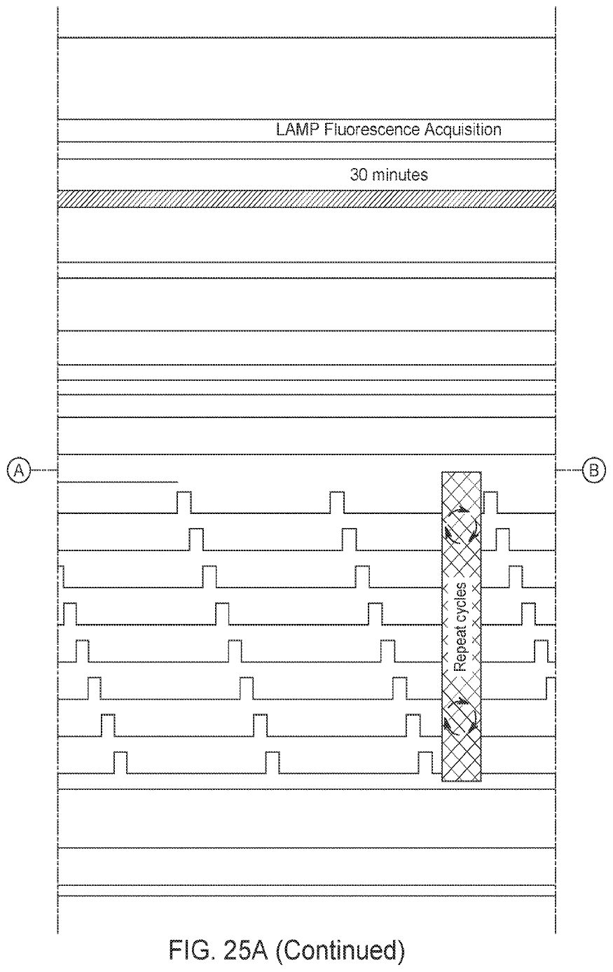

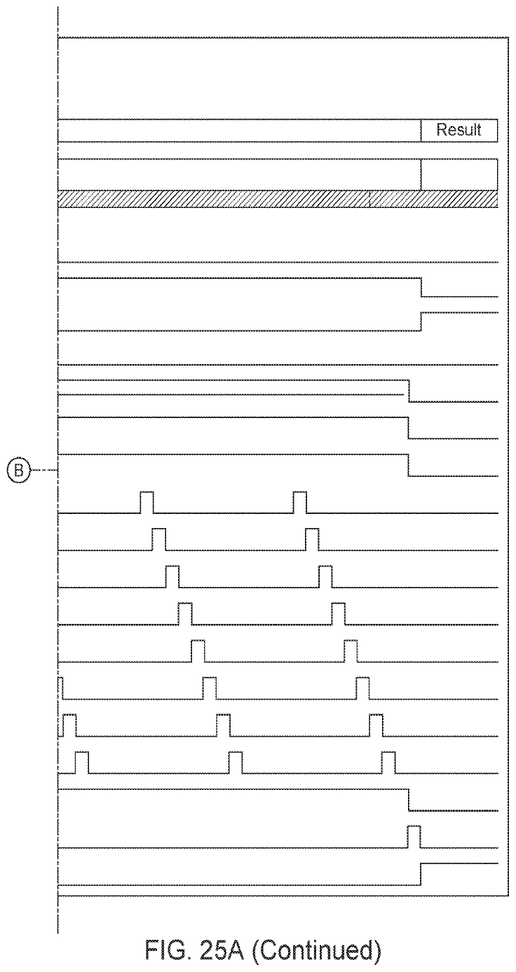

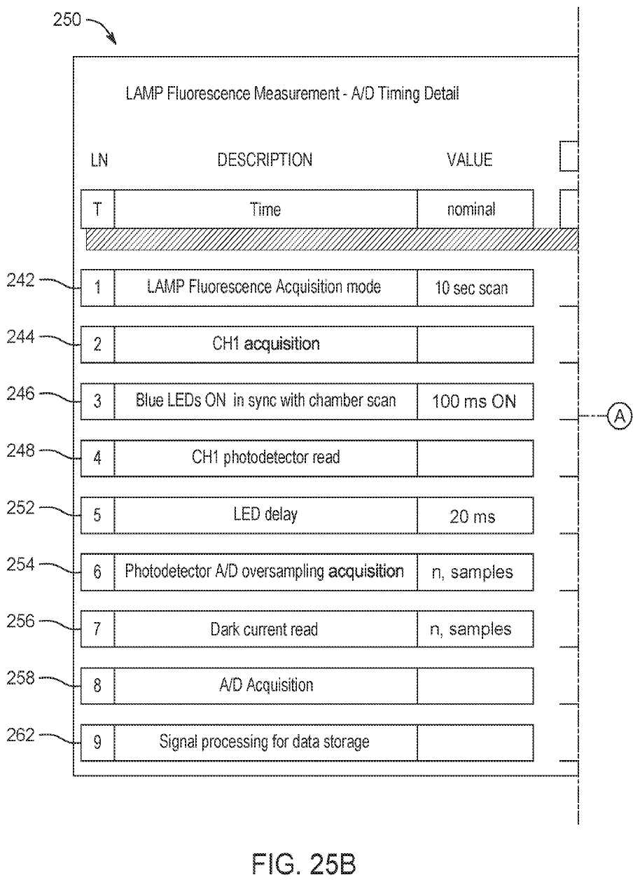

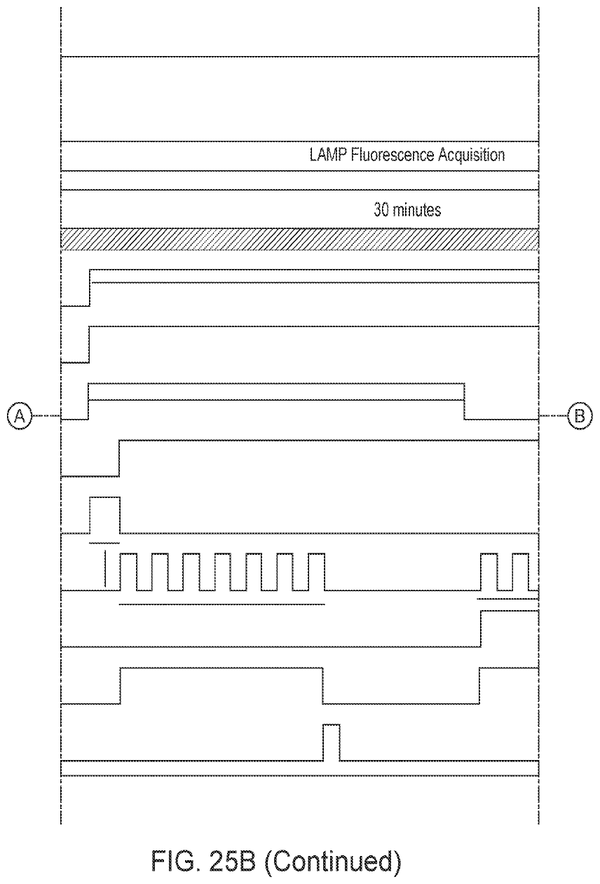

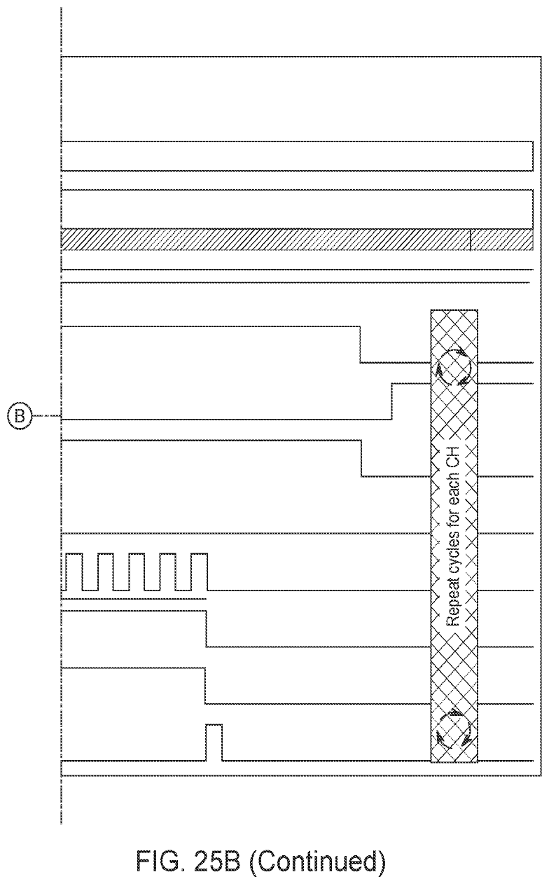

In some embodiments, a basic operating sequence 170 of a test run is shown in FIG. 22. The overall timing and sequence of a test run is shown in FIG. 23A and the timing and sequence of the “Cartridge ID” function is shown in FIG. 23B. The timing and sequence of the Cartridge ID function is shown in FIG. 24A and the timing and sequence of the “Fill Detect” function is shown in FIG. 24B. The timing and sequence of the optical fluorescence measurement during amplification is shown in FIG. 25A and the detailed timing and sequence of the optical fluorescence measurement is shown in in FIG. 25B.

Referring to FIG. 22, which illustrates an exemplary flow of operation according to the present embodiments, the basic operating sequence or method 170 may include the following steps: applying power 166 at the start step, performing a system boot sequence at step 168, and confirming reader ready state at step 172. The basic operating sequence or method 170 may include the following further steps: At step 188, preparing the cartridge 12 (by the user) with sample; at step 174, inserting the cartridge 12 into the reader 14; at step 176, detecting the cartridge identification; at step 178, clamping the reader and running the auto-start routine; at step 180, performing a test run; at step 182, performing the test or assay and displaying the results; at step 184, removing the cartridge 12 from the reader 14 and disposing of the cartridge 12; and at step 186, powering the reader 14 down (i.e., turning the reader 14 off).

In some embodiments, when configured for unconnected use, the system (i.e., reader 14) reports the test result to the user visually (e.g., each assay lights up for a positive result, as shown in FIG. 2A). In some embodiments, the reader 14 does not save detailed test data or transmit it to any other system or device.

In some embodiments, when configured for connected use, the reader 14 can transmit data via Bluetooth wireless radio. This data may include: Testing programs and parameters, firmware and software upgrades, user-entered information from cell phone applications, test results, raw test data, error codes, and test run metadata.

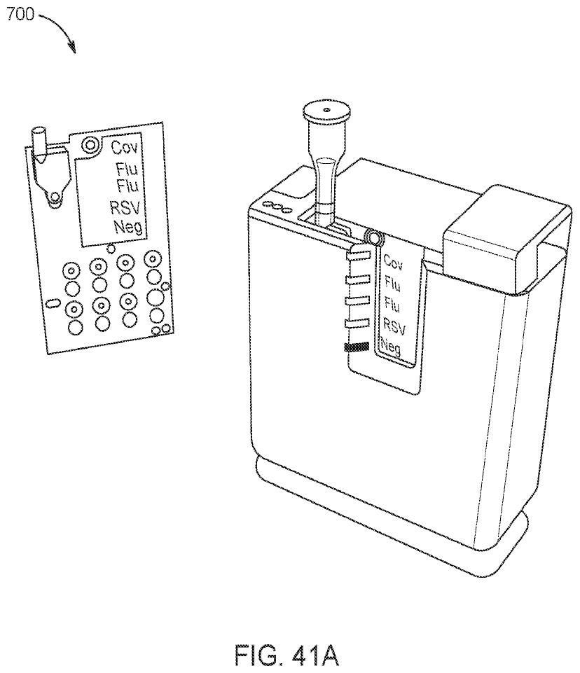

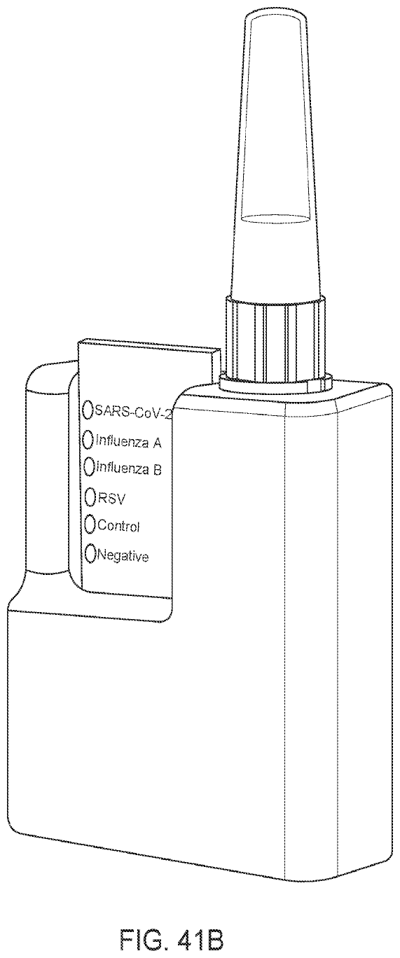

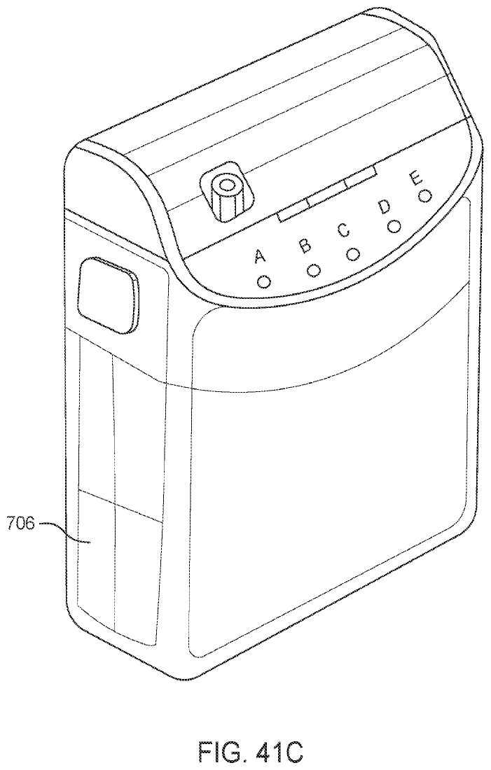

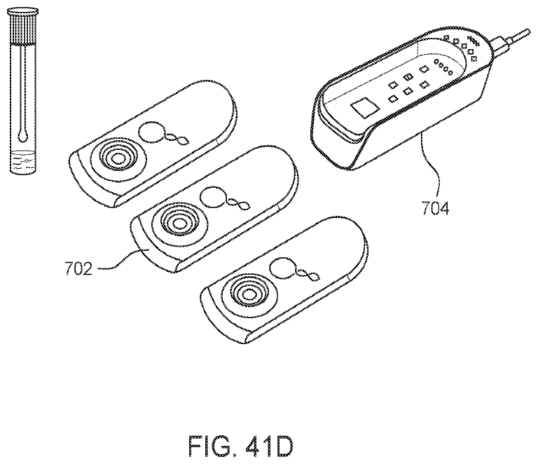

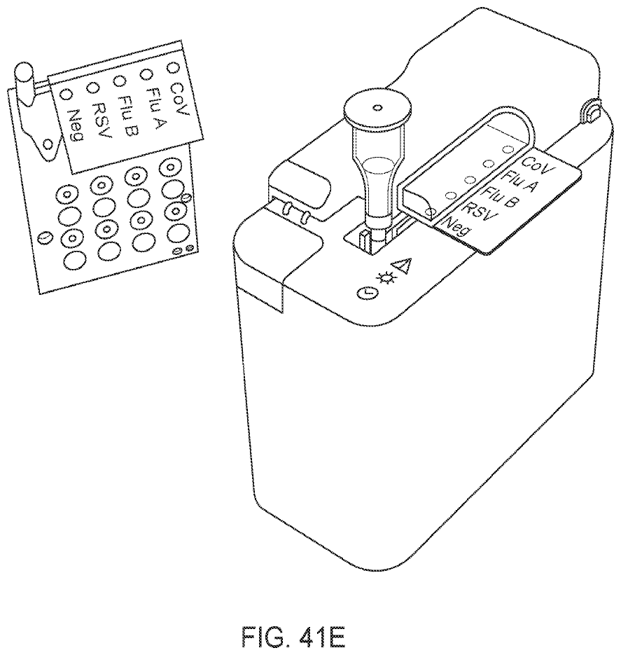

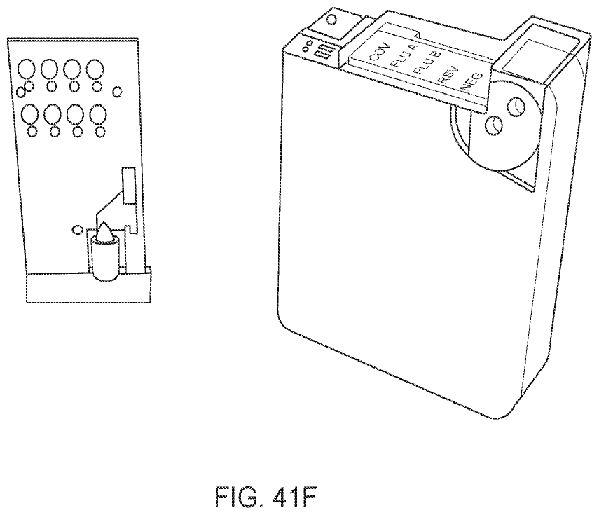

In some embodiments, a reader 14 may include an alternate form to e.g., optimize the usability and workflow. Viable industrial design concepts 700 for the systems are shown in FIG. 41. For example, the industrial design concepts may include a cartridge 702 configured to be horizontally inserted into a corresponding reader 704, as well as a reader 706 with a top surface that is at least partially angled, and at least partially flat (i.e., at least partially parallel with a horizontal plane).

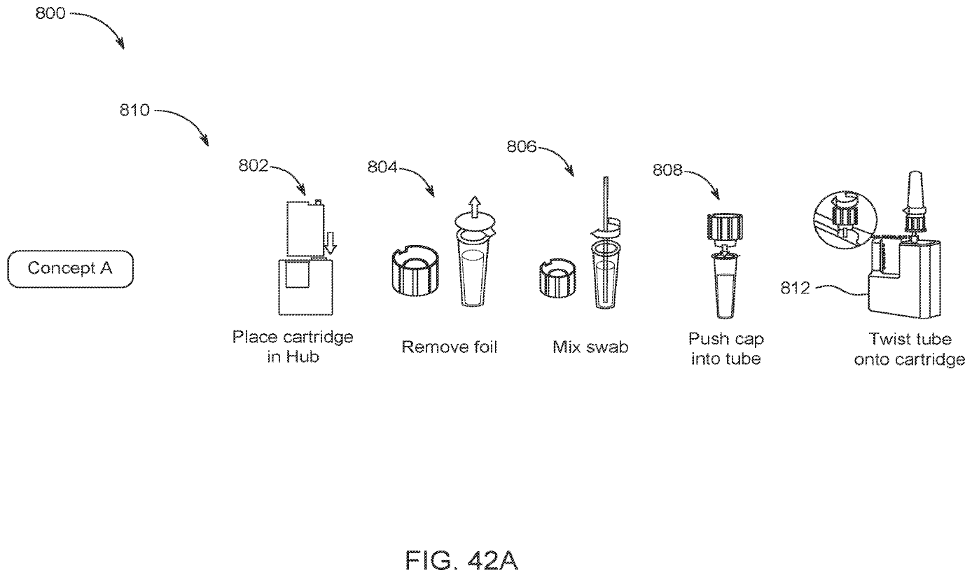

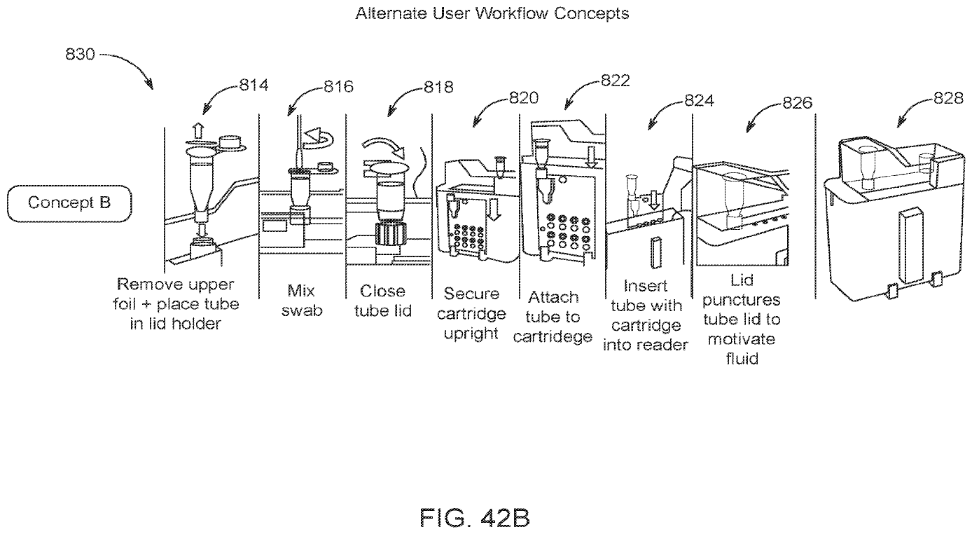

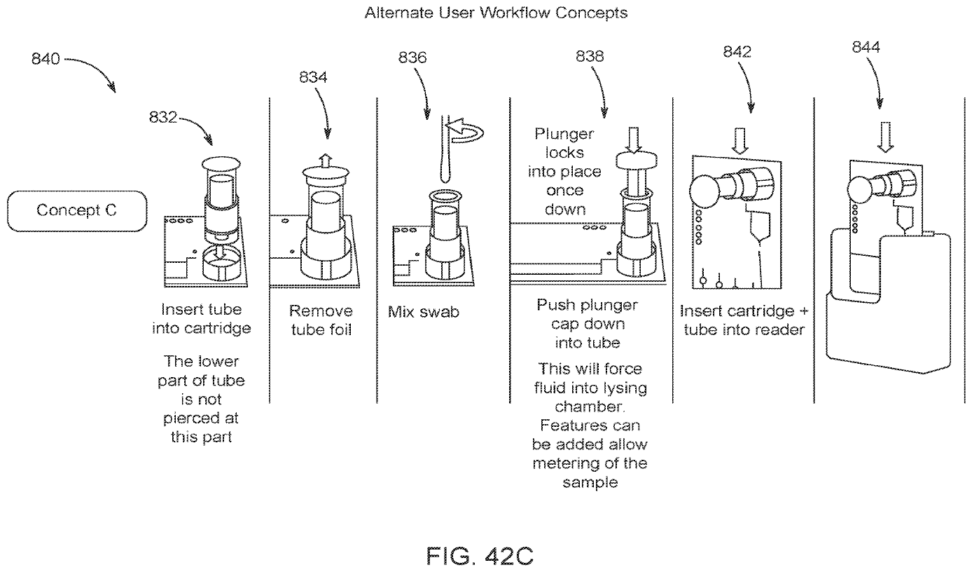

Alternate user workflows 800 are shown in FIGS. 42A, 42B, and 42C. A first alternate workflow 810 is shown as Concept A in FIG. 42A, and may include the following steps: At step 802, placing the cartridge into the hub; at step 804, removing foil that covers the vessel containing the buffer; at step 806, mixing the swab in the vessel; at step 808, pushing a cap into the tube; at step 812, twisting the tube onto the cartridge and proceeding with the assay according to the present description.

Referring to FIG. 42B, a second alternative workflow 830 is shown as Concept B, and may include the following steps: At step 814, removing upper foil and placing the tube in a lid on the holder (i.e., the reader); at step 816, mixing the swab in the vessel; at step 818, closing the tube lid; at step 820, securing the cartridge upright; at step 822, attaching the tube to the cartridge; at step 824, inserting the tube with the cartridge into the reader. At step 826, the second alternate workflow 830 may include puncturing the tube (i.e., by the lid) to motivate the fluid. At step 828, the second alternate workflow 830 may include proceeding with the assay according to the present disclosure.

Referring to FIG. 42C, a third alternate workflow 840 is shown as Concept C, and may include the following steps: At step 832, inserting the tube into the cartridge without piercing the lower part of the tube; at step 834, removing the tube foil; at step 836, mixing the swab in the vessel; at step 838, pushing the plunger cap down into the tube, thereby forcing fluid into the lysing chamber. In some embodiments, this may include the use of metering features. The third alternate workflow 840 may further include the following steps: At step 842, inserting the cartridge and tube into the reader; and at step 844, proceeding with the assay, after insertion into the reader is complete. In some embodiments, the system 100 may include a pierceable foil.

FIG. 23A shows an overall sequence timing 200 for the operation of the cartridge 12, reader 14, and system 10 in general (i.e., the timing sequence for a system complete run). For example, the overall sequence timing 200 includes LED status indicator timings 188, the sequence timing for cartridge insertion 190, cartridge identification 192, cartridge clamping 194, lysis heating 196, valve actuation 198, fill detection 202, LAMP heating 204, and results display 206, among other timings. The overall sequence timing 200 also includes a time bar 208 (FIG. 23A) illustrating the time corresponding to each step in the sequence. For example, cartridge insertion 190 begins around 1 minute and 25 seconds and the cartridge remains inserted for about 33 minutes until about 5 or 10 seconds before the end of the overall sequence. In another example, valve actuation 198 occurs during a roughly 10-second period following lysis heating 196.

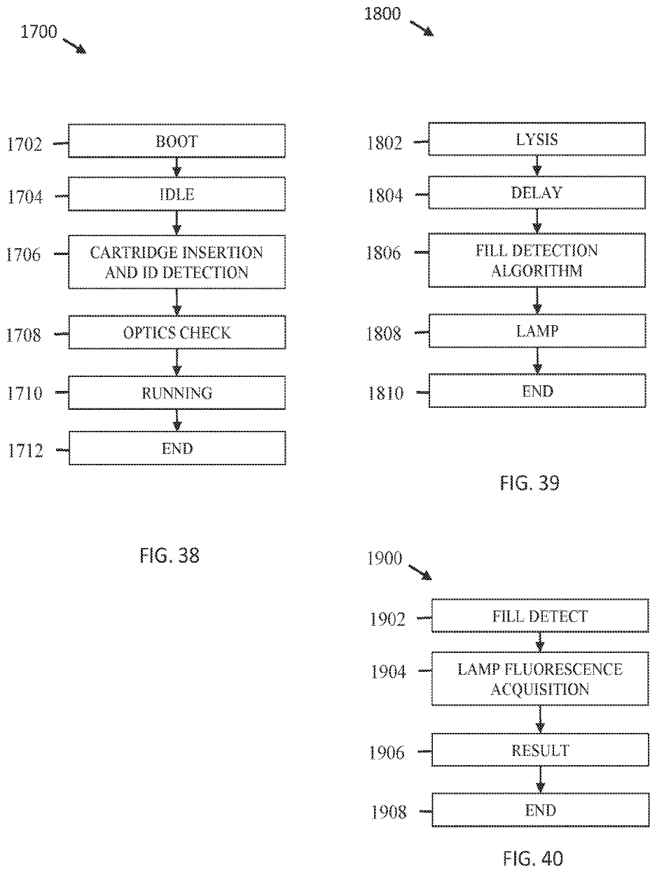

FIG. 23B illustrates the sequence timing 210 associated with the cartridge identification process or algorithm 1700. The cartridge identification process or algorithm 1700, at a high level, is illustrated in FIG. 38. The process 1700 may include the following steps: At step 1702, system booting; at step 1704, system idling; at step 1706, cartridge insertion and identification detection; at step 1708, performing one or more optics check; at step 1710, running the identification routine; and at step 1712, concluding with sending an internal communication to the microprocessor (within the reader) confirming the identification of the cartridge. As illustrated in FIG. 23B, the cartridge identification sequence timing 210 may include LED status indicator timings 188, cartridge clamping 194, cartridge position sensing 216 (i.e., sensing if the cartridge 12 is fully inserted), activation of the red LEDs 212, activation of the blue LEDs 218, and photodetector reads 214 from each of the eight (8) photodetectors (channels 1-8). As illustrated, the cartridge identification sequence timing 210 may include cycling through each of the 8 photodetector channels in sequence, and then repeating the cycles several times (for example, 3, 4, 5, 6, 7, 8, 9, and/or more than 9 time) to read the identification markings on the cartridge 12, as described herein.

FIG. 24A illustrates a cartridge identification sequence timing 220 in a high-speed mode, according to aspects of the present embodiments. At a high level, the high-speed mode cartridge identification sequence timing 220 follows the same process flow shown in FIG. 38 as the normal mode. The high-speed mode cartridge identification sequence timing 220 is similar to the sequence timing 210 shown in FIG. 23B with the exception that it includes a first period of sequential activation 222 of the first four (4) photodetector channels at higher frequency followed by a second period of sequential activation 224 of all eight (8) photodetector channels at the normal frequency, with the normal frequency being roughly half of the higher frequency.

FIG. 24B illustrates a fill detection algorithm sequence timing 230, according to aspects of the present embodiments. The fill detection algorithm 1800 or process, at a high level, is illustrated in FIG. 39. The fill detection algorithm or process 1800 may include the following steps: At step 1802, lysis (for example via lysis heating in the lysis chamber 94); at step 1804, a delay following the period during which lysis is occurring and, in some embodiments, concurrent with or prior to, the ball valve activation. As described herein, after ball valve activation, biological solution flows from the lysis chamber 94 into the reaction chambers 106 (during which time the biological solution is passively cooled). The process 1800 may further include the following steps: At step 1806, performing the fill detection algorithm; at step 1808, performing loop-mediated isothermal amplification (LAMP); and at step 1810, completion of the fill detect routine and communication, by the microprocessor, that the cartridge fill detect routine is complete (and fill has been detected or not detected). As shown in FIG. 24B, in some embodiments, fill detect 226 is initiated concurrent with ball valve activation (or actuation) and occurs at a frequency of 10 Hz (or from about 5 Hz to about 20 Hz, or from about 3 Hz to about 50 Hz, or from about 2 Hz to about 100 Hz, and/or other subranges therebetween). In some embodiments, actuation of the ball valve 98 occurs via mechanical means. In some embodiments, the system 10 includes thermal means (for example, the lysis area heating element 116) for actuating the ball valve 98. In some embodiments, concurrent with the initiation of fill detect 226, the fill detection algorithm sequence timing 230 may include monitoring singles 228 for fill activity.

FIG. 25A illustrates a LAMP fluorescence measurement timing sequence 240, according to aspects of the present embodiments. The LAMP fluorescence measurement timing sequence 1900 or process, at a high level, is illustrated in FIG. 40.

The process 1900 may include the following steps: At step 1902, performing (or completing) the fill detect routine; at step 1904, acquiring LAMP fluorescence (i.e., via the photodetectors and LEDs as described herein); at step 1902, displaying a result, following acquisition of the LAMP fluorescence; at step 1908, completion of the LAMP fluorescence acquisition routine and communication, by/to/within the microprocessor, that the LAMP fluorescence acquisition routine is complete. As shown in FIG. 25A (and FIG. 24B), in some embodiments, LAMP reaction heating 232 is initiated concurrent with the completion of fill detect 226. The timing sequence 240 may also include LAMP fluorescence acquisition mode 234 being initiated slightly after (for example, 1, 2, 5, 10, 20, or 30 seconds following) the initiation LAMP reaction heating 332. The timing sequence 240 may also include the initiation of curve algorithm monitoring of signals 236 concurrent with the initiation of LAMP fluorescence acquisition mode 234. Finally, as shown in FIG. 25A, the timing sequence 240 may also include the initiation of the call algorithm 238 upon (i.e., concurrent with) completion of each of the LAMP reaction heating 232, LAMP fluorescence acquisition mode 234, and curve algorithm monitoring of signals 236.

FIG. 25B illustrates a LAMP fluorescence measurement A/D timing sequence 250, according to aspects of the present embodiments. The LAMP fluorescence measurement A/D timing sequence 250 allows for oversampling, thereby enhancing the resolution of the fluorescence acquisition. The LAMP fluorescence measurement A/D timing sequence 250 in FIG. 25B is illustrated in terms of a single channel (i.e., for just a single reaction chamber 106 (CH1) as well as corresponding LEDs and photodetectors) but would apply with equal force to the any and all channels/reaction chambers 106 as well. The LAMP fluorescence measurement A/D timing sequence 250 may include initiating LAMP fluorescence acquisition mode 242 (for example, in 10 second scans) including acquisition at each of the reaction chambers 106 (for example, channel 1 (CH1) acquisition 244). The LAMP fluorescence measurement A/D timing sequence 250 may also include activating 246 blue LEDs in synchronization (for example, at a frequency of 100 ms) with the reaction chamber scans 244. Following a 20 ms LED delay 252, a CHI photodetector read 248 is performed, and photodetector A/D oversampling acquisition 254 is initiated (for example, in 10 ms intervals, repeated for several cycles, followed by a period (for example, 30 ms to 50 ms of inactivation, followed by a second and subsequent periods of activation in 10 ms intervals). During the second period of photodetector A/D oversampling acquisition 254, a dark current read 256 may be performed. A/D specific acquisition 258 may also be initiated in 50 ms intervals following the LED delay 252. Finally, the LAMP fluorescence measurement A/D timing sequence 250 may include performing signal processing 262 (i.e., to help facilitate data storage) at the completion of each 50 ms interval A/D specific acquisition 258.

### B. Cartridge Assembly

In some embodiments, the present disclosure provides a cartridge 12. In some embodiments, a cartridge 12 is assay specific (agnostic). In some embodiments, a cartridge 12 is a disposable cartridge. In some embodiments, a cartridge 12 is a low-cost disposable device that comprises a plurality of reagents for detecting a target nucleic acid.

In some embodiments, a cartridge 12 interfaces with a reader 14.

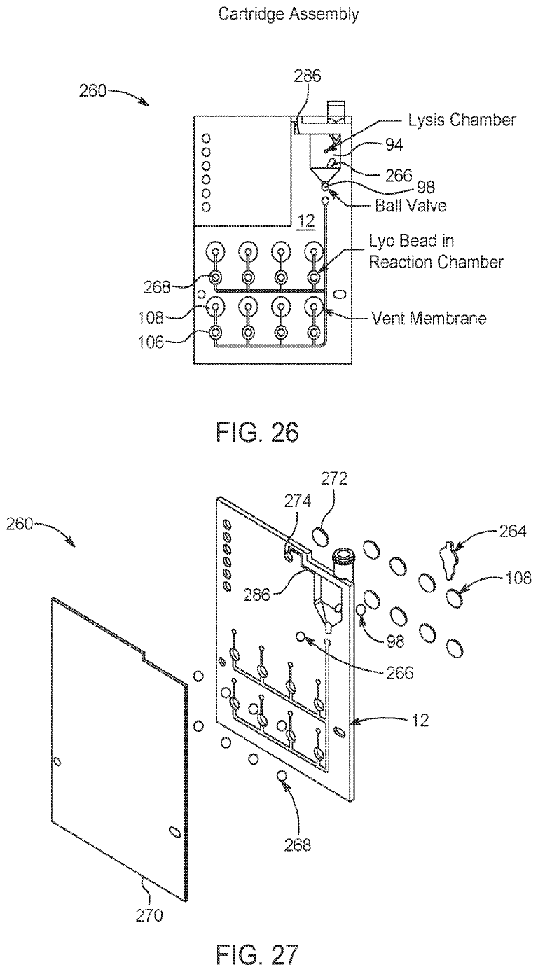

Referring to FIGS. 26 and 27, according to the present embodiments, a cartridge assembly 260 may include the cartridge 12, a plurality of vent membranes 108, a ball valve 98, a lyophilized lysis bead 266, a plurality of lyophilized PCR beads 268, and a front film 270. The lyophilized lysis bead 266 may be preloaded in the lysis chamber 94 while each of the plurality of (for example, eight (8)) lyophilized PCR beads 268 may be preloaded in the respective plurality of (e.g., eight (8)) reaction chambers 106. Accordingly, when the cartridge assembly 260 is fully assembled, each of the lyophilized reaction/PCR beads 268 and the lysis bead 266 are disposed between the cartridge 12 and film 270 such that they are partially encapsulated in the respective reaction chambers 106 or lysis chamber 94, with the film 270 holding them in place. In some embodiments, the lyophilized reaction/PCR beads 268 may include C7 FAM reporter, RS9 enzyme, DNA polymerase, dNTPs, buffer, and corresponding primers and guides thereof. In some embodiments, due to the material properties of the film 270, the film 270 acts as a heat spreader 128, thereby helping to enable heat transfer from the reaction area heating element 126 into the reaction chambers 106. As shown in FIG. 27, the cartridge assembly 260 may include an additional vent membrane 272 (i.e., a ninth vent membrane 272 in addition to the eight (8) vent membranes 108 used for venting each of the eight (8) reaction chambers 106 during fill). The additional vent membrane 272 may be used in connection with vent hole 274 for venting the lysis chamber 94 (i.e., allowing air to leave) when the cartridge 12 is initially filled via the lysis chamber 94. The cartridge assembly 260 may also include valve film 264 for holding the ball valve 98 in place and for facilitating actuation of the ball valve 98, which in some embodiments may include a 3 mm diameter. In some embodiments, the cartridge 12 may be molded (for example, via injection molding). In some embodiments, the cartridge 12 may be 3D printed or formed via machining, for example via CNC drilling. In some embodiments, the ball valve 98 may be coated with a lubricant (for example, a polymer coating (for example, a parylene coating)) to facilitate actuation of the ball valve 98 and to provide a moisture barrier to protect the ball valve 98 from corrosion or deterioration due to exposure to the biological solution (i.e., the saliva and buffer/reagent mixture). In some embodiments, the film 270 (i.e., front film 270) may include or be a microfluidic layer film with a thickness from about 0.003 inches to about 0.008 inches, for example, with a thickness of about 0.003, about 0.004, about 0.005, about 0.006, about 0.007, about 0.008 inches, and various subranges therebetween. In some embodiments, the front film 270 may include a polypropylene laminate with an adhesive layer. In some embodiments, the front film 270 (i.e., film layer 270) may be thermally, laser and/or ultrasonically welded to the cartridge 12.

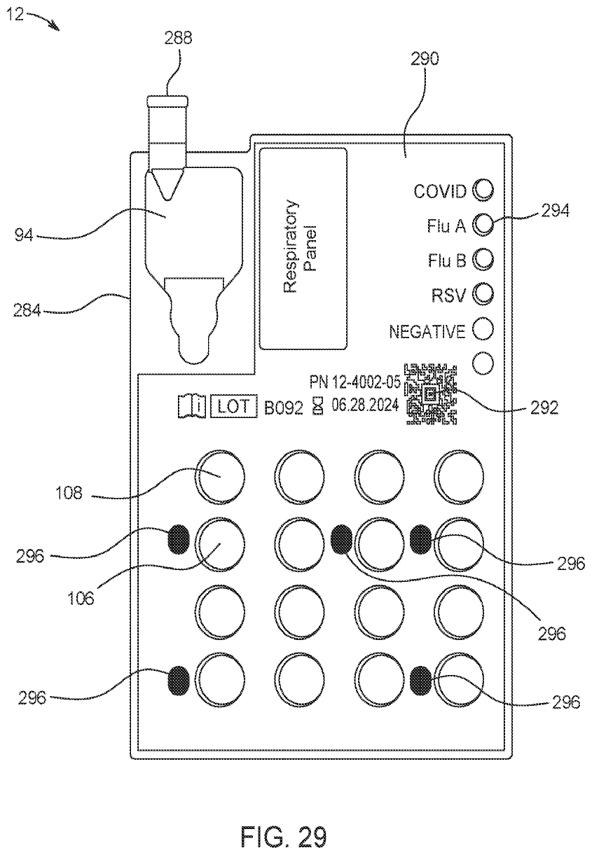

In some embodiments, a cartridge 12 comprises an injection molded cartridge body 284. An exemplary cartridge body 284 is shown in FIGS. 6 and 29. In some embodiments, a cartridge body 284 is a plastic cartridge body. In some embodiments, a plastic cartridge body is made of a plastic polymer selected from the groups consisting of polycarbonate, polymethylmethacrylate (PMMA), cyclo-olefin polymer (COP), and cyclo-olefin copolymer (COC).

In some embodiments, one or more reaction chambers 106 are molded into the cartridge body 284. In some embodiments, 1 to 30 reaction chambers 106 (for example, 2 to 20, 4 to 16, 5 to 15, 6 to 12, 7 to 10, or about 8) are molded into the cartridge body 284. In some embodiments, reaction chambers 106 are fluidly connected to a sample lysis chamber 94 via a valve (not shown). In some embodiments, multiple reaction chambers 106 are molded into the plastic body 284. An exemplary cartridge body 284 comprising 8 reaction chambers 106 is depicted in FIG. 29; the number of reaction chambers can range from 1 to 30.

In some embodiments, a cartridge 12 comprises one or more reaction chambers 106. In some embodiments, a cartridge comprises two or more reaction chambers 106. In some embodiments, a cartridge comprises three or more reaction chambers 106. In some embodiments, a cartridge comprises four or more reaction chambers 106. In some embodiments, a cartridge comprises five or more reaction chambers 106. In some embodiments, a cartridge comprises six or more reaction chambers 106. In some embodiments, a cartridge comprises seven or more reaction chambers 106. In some embodiments, a cartridge comprises eight or more reaction chambers 106.

In some embodiments, the reaction chamber 106 volume is about 5 μL to about 100 μL (such as about 10 μL to about 60 μL, such as about 20 μL to about 50 μL, such as about 40 μL). In some embodiments, a reaction chamber 106 comprises a lyophilized reagent. In some embodiments, a reaction chamber 106 is sealed by a film layer 270 (shown in FIG. 27).

In some embodiments, one or more reaction chambers 106 comprise lyophilized reagents. In some embodiments, a detection system 10 comprises lyophilized beads. In some embodiments, a lyophilized bead 268 (shown in FIG. 27) is used to verify filling of each reaction chamber 106. A change in fluorescence that occurs when a lyophilized bead 268 is rehydrated can be measured and used to verify filling of the reaction chamber 106 with a sample.

In some embodiments, the present disclosure provides a disposable cartridge 12 for detecting a target nucleic acid, the disposable cartridge 12 comprising a lysis chamber 94 (for example, a first chamber or first heating zone) for receiving a sample comprising the target nucleic acid; a reaction chamber 106 connected via a first channel 104 (shown in FIG. 9) to the lysis chamber and connected via a second channel 286 (for example, a vent channel 286, shown in FIGS. 26 and 27) to a first vent hole 274.

The cartridge 12, in some embodiments, is a single-use element that receives the sample, contains the dry reagents, and perform the assay inside a reader 14. The cartridge 12 is sealed by a cap 20 (shown in FIG. 1) after sample transfer, and contains all of the reagents and reaction products during and after the test. The cartridge 12 contains the dry reagents (in lyophilized bead 255, 268 form) within the reaction chambers and the lysis chamber (FIG. 27).

Referring again to FIG. 29, in some embodiments, a cartridge body 284 comprises a sample lysis chamber 94. In some embodiments, a cartridge body 284 comprises a sample inlet port 288. In some embodiments, a cartridge body 284 comprises a label 290. In some embodiments, a cartridge body 284 comprises a unique device identification (UDI) barcode 292. In some embodiments, a cartridge body 284 comprises a result display 294. The vent membranes 108 are also illustrated in FIG. 29, each being disposed on the cartridge body 284 and spaced vertically above each respective reaction chamber 106 to which it is fluidly coupled.

In some embodiments, a cartridge assembly 260 includes a fluidic ball valve 98, hydrophobic vents 101, 272, and two cover films 264, 270 (FIG. 27). In some embodiments, the cartridge body 284 is the primary component and is constructed primarily of an optically-clear polymer (such as polycarbonate, COC, COP, etc.).

Alternately, the cartridge body 284 can be constructed from optically clear material only in the reaction chamber 106 areas, and another material (i.e., a non-optically clear material) elsewhere (such as using a 2-shot injection molding process, insert-molding process, or other assembly method).

In some embodiments, a cartridge 12 can be in an alternate form to e.g., optimize the usability and workflow. Viable industrial design concepts for the systems are shown in FIG. 41. Alternate form factors for the cartridge 12, sample collection, and user workflow are shown in FIGS. 42A, 42B, and 42C.

In some embodiments, a cartridge 12 comprises a Cas enzyme, a probe and a guide.

In some embodiments, a cartridge 12 contains a label 290 indicating the test menu type and the assay and/or analytical result.

### Cartridge ID Optics

In some embodiments, a reader 14 detects the cartridge 12 type using an optical reading of the cartridge device label, as explained herein. For example, cartridge 12 type may be detected using either a static ID procedure or a dynamic ID procedure.

### Static ID

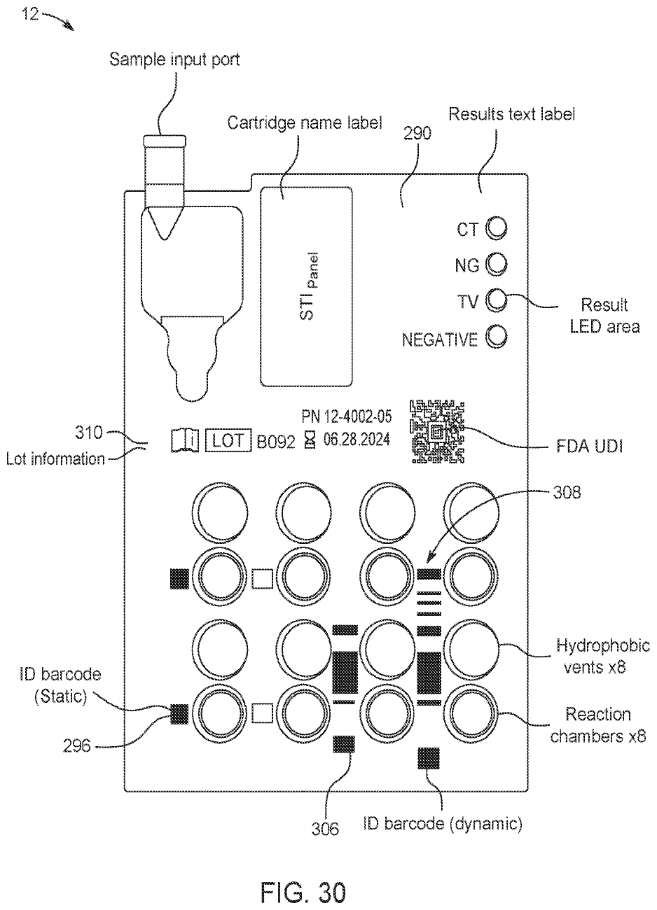

In some embodiments, static ID mode is used as a barcode to identify what type of cartridge 12 is being used (i.e., with the corresponding reagents, buffers, and/or lyophilized beads) such that the reader 14 may run the appropriate test sequence(s) and routine(s) for the type of assay(s) that are being performed. In some embodiments, static ID mode is used to identify a specific cartridge 12, such as a specific test panel. In some embodiments, a static identification label 290 may include printed optical targets 296 (or label marks 296, or identification markers 296). In some embodiments, optical targets 296 (or printed marks 296) change the reflection of an illumination LED whose wavelength is beyond the long-pass detector cutoff. Exemplary optical targets 296 (or printed marks 296) are shown printed as black circles in FIG. 29 and black rectangles in FIG. 30. In some embodiments, a photodetector 144, 162 located adjacent to the illumination LED (e.g., 158, 146) detects the presence of a label mark 296 (or printer mark 296). The corresponding array of optical targets 296 (or printed mark 296) comprise a barcode or unique signature. The reflection measurement sequence is shown in FIGS. 23B and 24A.

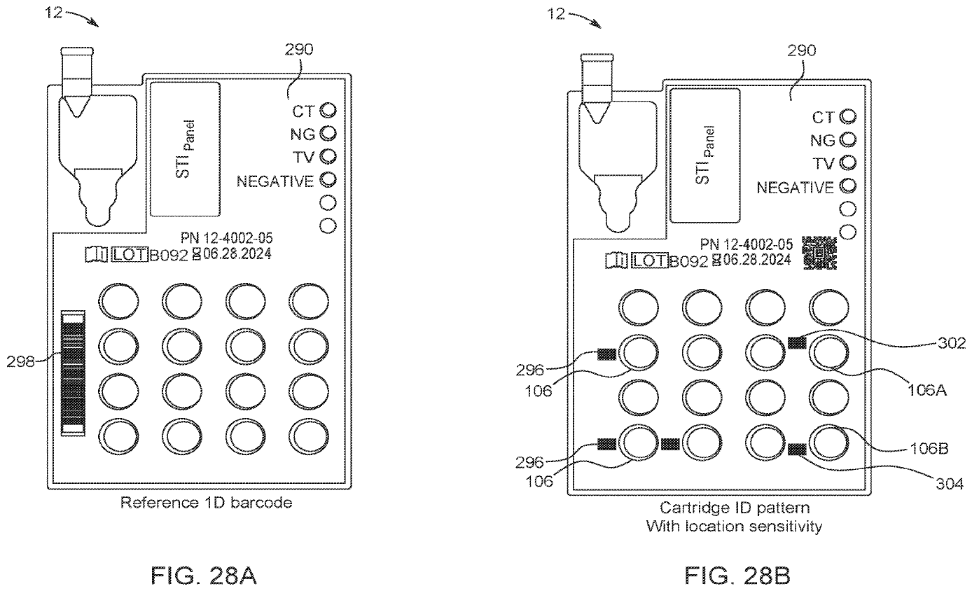

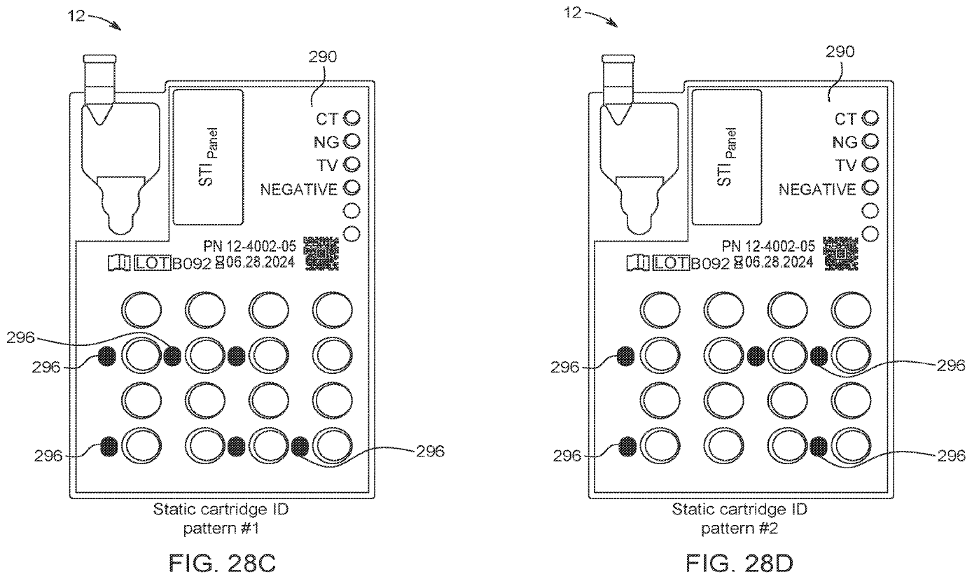

In some embodiments, a cartridge 12, in connection with the reader 14, uses one or more specific label patterns to identify a specific cartridge 12. An exemplary label pattern for respiratory diseases is show in FIG. 29. FIG. 28 shows exemplary label embodiments including a traditional 1D barcode reference method (FIG. 28A) using a conventional barcode 298 (which may be read in connection with a conventional bar code scanner).

### Position Sensing

In some embodiments, a cartridge 12 incorporates a position sensing feature 302, 304. An example thereof is shown in FIG. 28B. A position sensing feature 302, 304 is helpful and useful in determining that the cartridge 12 is appropriately and fully inserted into the reader 14. Such a feature 302, 304 may be employed as a check (upon the cartridge 12 being inserted) that a positive condition is met. In some embodiments, a position sensing feature 302, 304 is enabled by optical targets 302, 304. In some embodiments, a sensing feature 302, 304 is a target located slightly above the centerline of the adjacent reaction chamber 106. In some embodiments, a sensing feature 302, 304 is slightly below the horizontal axis centerline of its adjacent reaction chamber 106. Examples thereof are shown in FIG. 28B. In operation, the LED (e.g., red LED 158) in FIGS. 19A and 19B is located on the centerline of the reaction chamber 106 and photodiode 144 (or phototransistor 162). For the optic module 140 to determine that the cartridge 12 is fully inserted in the proper position, the system 10 measures a reflection from both optical targets 302, 304). If the cartridge 12 is located too high or too low (vertical axis in the figure), only one condition is met. Hence an insertion fault can be detected. The configuration and system are capable of measuring position to a resolution of about 0.010 inches when properly gain calibrated.

Referring again to FIG. 28B, a first position sensing feature 302 is located slightly above the centerline of an adjacent reaction chamber 106A. Similarly, the cartridge 12 (in connection with label 290) may include a second position sensing feature 304 located slightly above the centerline (i.e., horizontal centerline) of an adjacent reaction chamber 106B. Each of the first and second position sensing features 302, 304 may include, comprise, or be a geometric shape of one shade or color on top of a background surface or area of the label 290 of a different shade or color such that contrast between the position sensing features 302, 304 and the background is visible or detectable via the photodiode 144 (or phototransistor 162). For example, in the embodiment of FIG. 28B, the position sensing features 302, 304 include black rectangles on a white background. In the embodiment of FIG. 28C and FIG. 28D, the position sensing features 302, 304, as well as the printed optical targets 296 (or label marks 296, or identification markers 296) may include a circle, an octagon, a square with rounded corners, and/or other suitable shapes. In order for a “position correct” signal to be processed by the microprocessor (i.e., in the reader), the first position sensing feature 302 must be identified as being above the reference reaction chamber 106A centerline while the second position sensing feature 304 must be identified as being below the reference reaction chamber 106B centerline. If both the first and second position sensing features 302, 304 are either below or above the reference reaction chamber 106A, 106B centerlines, the cartridge 12 will not be sensed as in the correct position, and the additional algorithms and routines will not proceed. In contrast to the relative positions of the first and second position sensing features 302, 304, and still referring to FIG. 28B, the other printed optical targets 296 (or label marks 296, or identification markers 296) may be aligned with the respective centerlines of adjacent reaction chambers 106.

FIGS. 28C and 28D illustrate cartridges 12 with two different static ID patterns. In the cartridge of FIG. 28C, the other printed optical targets 296 (or label marks 296, or identification markers 296) are in different positions than those of FIG. 28D. For example, in the embodiment of FIG. 28C, the cartridge includes 6 identification markers 296, while the embodiment of FIG. 28D includes 5 identification markers 296. In addition, some of the identification markers 296 in FIG. 28C are in different positions than those of FIG. 28D, and vice versa. With eight (8) potential positions for the identification markers 296, and anywhere from 0 to 8 identification markers 296 possible for inclusion on a given cartridge, there are 8 factorial (plus 1) or 40,321 potential identifications that can be encoded on the label 290 using the identification markers 296.

### Dynamic ID

In some embodiments, a cartridge 12 may be identified in dynamic ID mode, i.e., while the cartridge 12 is in motion as it is being inserted into a reader 14. In some embodiments, a cartridge 12 comprises an identifying ID label 290. An exemplary identifying ID label is shown in FIG. 30. In some embodiments an identifying ID label 290 comprises a first barcode optical target pattern 306 and/or a second barcode optical target pattern 308, in addition to one or more static identification markers 296.

In operation, LEDs (e.g., red LEDs 158 as shown in FIGS. 19A and 19B) are constantly illuminated during the cartridge insertion step. In some embodiments, this is enabled via software control. During the insertion (vertical downward motion of the cartridge 12 on the vertical axis) barcode optical target patterns 306, 308 of FIG. 30 are illuminated by LEDs (e.g., red LEDs 158), and the resulting optical signature is detected by the photodetectors 144, 162 (e.g., as shown in FIGS. 19A and 19B), thereby forming a dynamic detection system that senses the identification of the cartridge 12 while it is moving using the photodetectors 144, 162 already present in the reader 14.

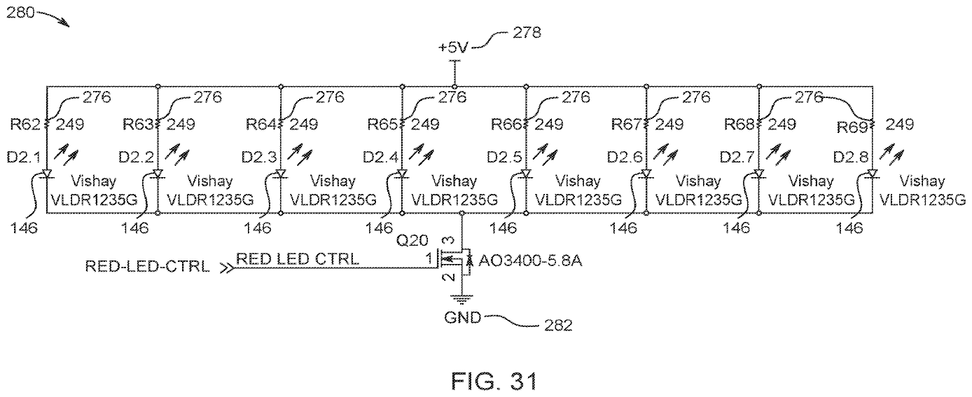

An exemplary design schematic of the LED drive circuit 280 and components is shown in FIG. 31. In some embodiments, the LED drive circuit 280 may include the eight (8) LEDs 146 (or 158 as the case may be) arranged in parallel between a voltage source 278 (for example, a 5-volt source) and ground 282, each of the LEDs 146 being positioned downstream of a corresponding resistor 276. In some embodiments, adjacent photodetectors 144, 162 above the reaction chambers 106 measure the reflection intensities. In some embodiments, photodetectors 144, 162 are multiplexed at a frequency high enough to resolve the velocity of the cartridge 12 insertion. Typically, the upper row of reaction chamber 106 and detectors 144, 162 is used as that row senses the label barcode 306, 308 first (upon insertion from the top). In some embodiments, first barcode optical target pattern 306 and second barcode optical target pattern 308 are the same. In some embodiments, first barcode optical target pattern 306 and second barcode optical target pattern 308 are different. It is further realized that one pattern 306 or 308 could be a timing marker of equally spaced marks to measure the cartridge 12 velocity should it prove to make the software identification easier. Stated otherwise, one of the patterns (i.e., either first barcode optical target pattern 306 or second barcode optical target pattern 308) could be the same each time and therefore used to assess how quickly the cartridge 12 is being inserted into the reader 14. The other pattern (i.e., either first barcode optical target pattern 306 or second barcode optical target pattern 308) could then be used as an actual identifier representing the type of cartridge 12 being inserted. In some embodiments, the insertion speed information from the first pattern (i.e., the timing pattern or barcode, for example, first barcode optical target pattern 306) can be used to calibrate the optical signature from the second pattern (i.e., the identification barcode, for example, second barcode optical target pattern 308) such that proper spacing between bars of the barcode can be adjusted and/or corrected as needed to ensure accurate interpretation of the cartridge identification. The identification markers 296, position sensing features 302, 304, and barcode optical target pattern 306, 308 (i.e., shown in FIGS. 28-30 and 32) allow cartridges 12 to be both properly identified by the reader 14 and positioned correctly within the reader 14, without the need for a barcode scanner, by making use of photodetectors 144, 162 that are already present in the reader 14.

Referring still to FIGS. 28-30 and 32, once the identification of a cartridge 12 has been determined, the reader 14 will then automatically execute the routines and sequences corresponding to the specific cartridge 12 in question. The cartridge 12 identification dictates which set of internal (via software) sequences are run by the reader. In some embodiments, the reader 14 is Wi-Fi (i.e., network or internal) enabled such that updates to the software may be implemented (i.e., remotely uploaded to, and installed on the reader 14) to allow new and/or updated assays to be run on the reader 14 (i.e., using the same reader 14 hardware), in some cases, with newly developed disposable cartridges 12 (for example, to allow for strain-specific assays (i.e., new strains) of various viruses to be run via the same underlying system).

### Cartridge ID With Ambient Light Blocking Label

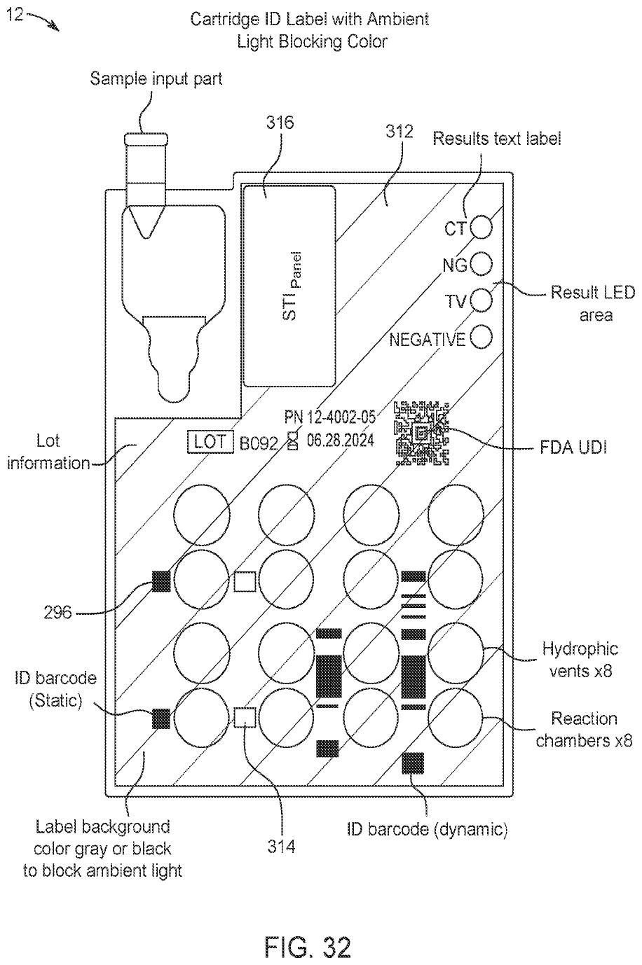

In some embodiments, a cartridge 12 comprises a label 312 with a colored or shaded background. In some embodiments, a cartridge 12 comprises a dark or opaque label 312 background. An exemplary dark or opaque label background 312 is shown in FIG. 32.

The shaded or colored label 312 provides ambient light blocking properties. When utilized, the barcode optical target features 296 may be alternate colors, transparent, or white 314 in order to enable reflection changes that the photodetector 144, 162 can measure. The cartridge 12 may also include a cartridge name label 316 (i.e., a second, different label from the first label 290, 312).

In some embodiments, an LED used for fluorescence excitation has certain spectral emission characteristics. FIG. 34 shows an emission spectrum of a fluorescence excitation LED. In some embodiments, it is important that an illumination pattern contains a narrow beam for maximum energy transfer in exciting the reaction chamber 106 fluid.

Typical half-angle emission patterns are in the range of 5 to 10 degrees for typical total viewing angles of 20 degrees. In some embodiments, an LED may be an LED XZCBD78W by SunLED. There are several alternate part numbers that may also be used that are in the categories of surface mount, InGaN emitting material, narrow cone angle beam pattern, and high intensity output.

In some embodiments, a cartridge 12 comprises a filter. In some embodiments, an optic module comprises a filter. In some embodiments, a filter is a fluorescence long-pass filter. In some embodiments, a filter has a centerline cutoff of about 520 nm. In some embodiments, a fluorescence long-pass filter is utilized to measure a FAM-labelled reaction fluid. The optical module 140 uses this and/or other gel, film, or plastic filters instead of a traditional dichroic glass filter, which may be too expensive to be practically useful in the reader 14 and product concept developed in connection with the present disclosure. In some embodiments, a filter is a Kodak Wratten 12 filter with a centerline cutoff of about 520 nm. In some embodiments, a separate filter may be disposed on the outer surface of each reaction chamber dome. In some embodiments, a filter may be disposed over more than one reaction chamber.

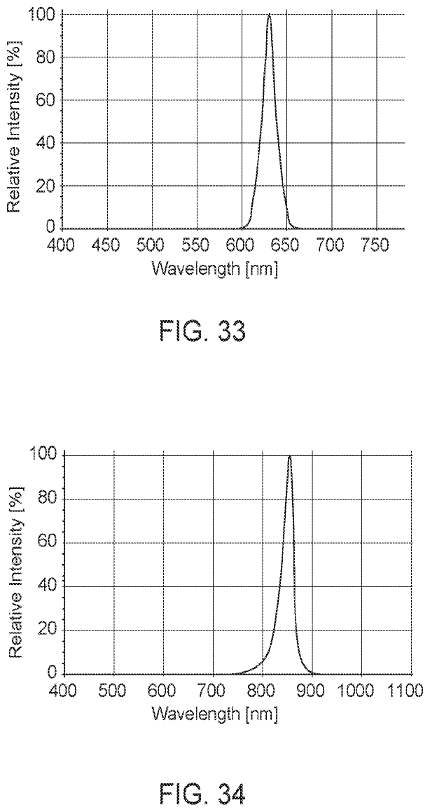

FIG. 33 shows an emission spectrum of a red illumination LED. In some embodiments, an illumination pattern contains a narrow beam for enhanced performance. Typical half-angle emission patterns are in the range of 5 to 10 degrees. In some embodiments, a red illumination LED is a LED VLDR1235G by Vishay Semiconductor, and may emit light in a range from about 600 nm to about 660 nm wavelength.

FIG. 34 shows the emission spectrum of an alternative illumination LED emitting light with wavelengths in the near-infrared in a range from about 730 nm to about 900 nm, and centered at about 865 nm. In some embodiments, an illumination pattern contains a narrow beam for optimum performance. In some embodiments, the illumination near-infrared spectrum is within a range not visible to the human eye (for example, in a range from about 730 nm to about 900 nm and/or at wavelengths above 700 nm). Typical half-angle emission patterns are in the range of 5 to 10 degrees. In some embodiments, an LED is a LED SFH 4059-QS by OSRAM Opto Semiconductors GmhH. In some embodiments, the photodetectors 144, 162 described herein include dual mode photodetectors or tri mode photodetectors (i.e., dual/tri mode photodiodes 144 and/or dual/tri mode phototransistors 162) capable of being calibrated to measure wavelengths in the ranges shown in FIG. 33 (i.e., 600 nm to 660 nm), FIG. 34 (i.e., 730 nm to 900 nm), and/or FIG. 17 (i.e., 400 nm to 640 nm). Accordingly, in some embodiments the photodetectors 144, 162 described herein may be calibrated to measure wavelengths in a range from about 400 nm to about 900 nm.

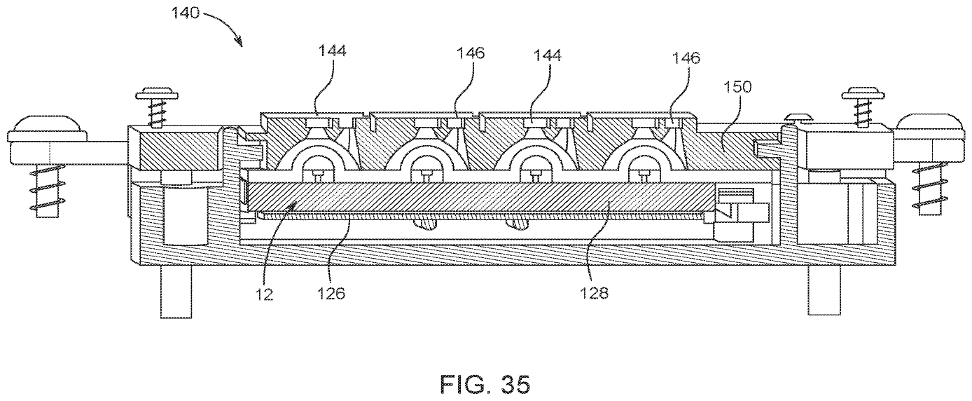

FIG. 35 shows an embodiment of the prototype design. FIG. 35 illustrates a cut-away side view showing the cartridge 12 and its interface with the heating element 126, heater spreader 128, and the optical module 140.

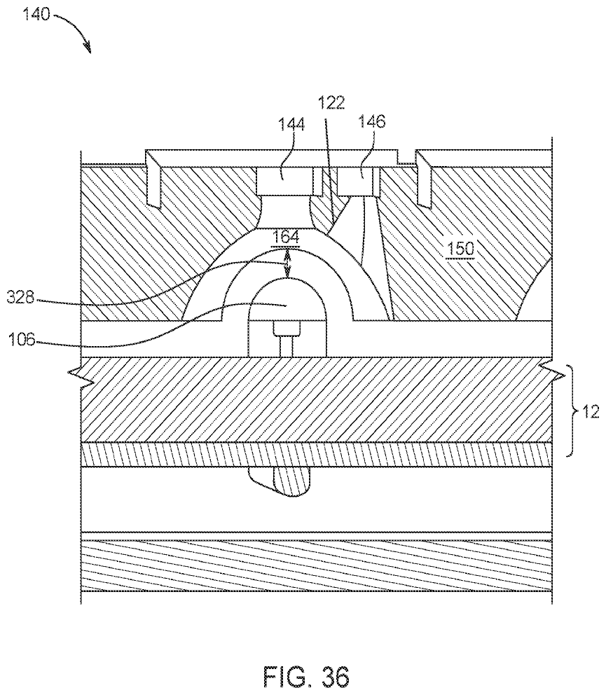

FIG. 19A shows a detail of the optic module PCBA layout. Enhanced optical signal-to-noise ratio is achieved by placement of the optical components with respect to the cartridge reaction chamber 106 shown in FIG. 35. In some embodiments, and as shown in FIG. 36 (magnified view of a portion of FIG. 35), a photodiode 144 is located directly above the reaction chamber 106 volume such that maximum photo energy is collected from the fluorescent emission. In some embodiments, a 460 nm blue excitation LED 146 is placed as close as practicable so that maximum output energy from the narrow (e.g. 20 degree cone angle) beam enters and excites the reaction mixture in the reaction chamber 106. An optical shield 122 may be used to prevent stray excitation light from entering the photodiode 144 from the side or via unwanted reflections. In some embodiments, a photodiode 144 incorporates a filter 160 (shown in FIGS. 19A and 19B) on the top surface via an optical epoxy or similar assembly method.

In the embodiments shown in FIGS. 35 and 36, the photodetector may include a photodiode 144 as shown or a photo transistor 162. As shown in FIG. 36, the optical module 140 may include a recessed portion 164 with concave contouring to match the outer shape of the reaction chamber 106 domes (or hemispheres). The recessed portion 164 may be disposed in the printed circuit board (PCBA) 150. When the cartridge 12 is inserted into the reader 14, in some embodiments it is inserted vertically (i.e., in a downward direction, i.e., out of page in the view of FIGS. 35 and 36), and then is pushed laterally (i.e., upward in the view of FIGS. 35 and 36) such that the reaction chambers 106 are close to the optical module 140, and such that the heating elements 116, 126 and heat spreaders 118, 128 are brought close to or in contact with the cartridge 12), as described in connection with the description of FIGS. 13 and 14. Accordingly, when the cartridge 12 is pushed laterally via the mechanical assembly 50, the domes (or hemispheres) of each of the reaction chambers 106 is brought within each of the corresponding recesses 164 disposed in the PCBA 150. Once the cartridge 12 is positioned correctly within the reader 14 such that the dome of the reaction chamber 106 is at least partially protruding into the arc of the recessed portion 164, as shown in FIG. 36, a spacing 328 of about 1 mm to about 4 mm (for example, from about 2 mm to about 3 mm) is maintained between the top of the dome of the reaction chamber 106 and the center point of recessed portion 164.

In some embodiments, the dome (or hemisphere) of the reaction chamber 106 is concentric within the recessed portion 164, when inserted correctly. In some embodiments, the dome (or hemisphere) of the reaction chamber 106 is composed of a transparent polycarbonate such as COC and/or COP (cyclic olefin copolymer and cyclic olefin polymer). The arrangement described herein with respect to FIGS. 35 and 36 has been shown to result in an enhanced signal-to-noise ratio with respect to fluorescence detection. For example, the present embodiments to not include or require a lens, due to the close reaction chamber 106 placement to both the light sources (i.e., LEDs 146, 158) and the photodetectors 144, 162. Accordingly, the optical assembly 140 of the present embodiments may be less costly and less complex than other comparable systems due to not needing an optical lens.

In the embodiment of FIG. 36, the optical shield prevents light emitted by the LED from being directly detected by the photodetector. After activating the biological solution in the reaction chamber, the photodetector detects the activation solution. Because of the location of the photo detector in close proximity to the reaction chamber, an optical lens is not required. In some embodiments, a first LED 158 (e.g., red LED) in FIGS. 19A and 19B is placed to one side of a photodiode 144, while a second LED (e.g., a 460 nm (blue) LED 146) is placed to another adjacent side of the photodiode 144, such that the first LED 158, the photodiode 144, and the second LED 146 form a 90-degree angle. In some embodiments, an LED (e.g., red LED 158) placement is also in-line horizontally with the photodiode 144 and placed such that is it between adjacent reaction chambers. This enables the printing of a barcoded optical target 296 on a label 290 in an available space on the cartridge 12 surface which does not interfere with the hydrophobic vents 108 (i.e., vent membranes 108). The location of the red LED 158 also enables and permits alignment for dynamic ID sensing during cartridge 12 insertion, which may be advantageous.

In some embodiments, a photodiode 144 is a photodetector from TEMD5020X01 by Vishay Semiconductors. It is noted that other photodiodes 144 or phototransistors 162 (and associated components) may also be used as the photodetector 144, 162 element as long as the size and cost constraints are met, which improves feasibility for at-home testing.

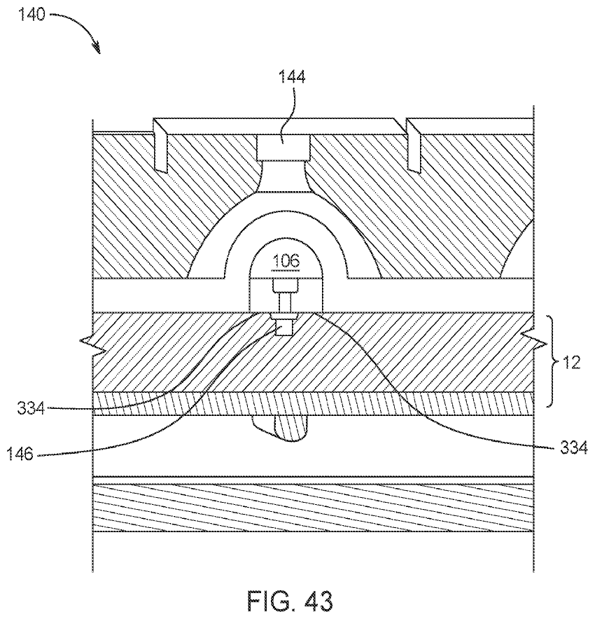

FIG. 43 illustrates a side view of the reaction chamber assembly in an alternate configuration, according to aspects of the present embodiments. In the embodiment of FIG. 43, at least one of the LEDs (for example, the blue LED 146) is integrated into the cartridge 12 such that is it is positioned immediately under the reaction chamber 106 to enhance excitation of the nucleic acids contained therein. The presence of the LED 146 disposed within the cartridge 12 would require a space, hole, and/or void in the cartridge 12, potentially diminishing the transfer of heat into the reaction chamber 106. In some embodiments, the LED 146 includes a smaller diameter than that of the reaction chamber 106, thereby allowing a ring 334 surrounding the LED 146 where the reaction chamber 106 directly contacts the cartridge 12 material, to encourage heat transfer into the reaction chamber 106. Alternatively, or in addition, inefficient LEDs may be used (i.e., LEDs that draw additional current and give off more thermal energy) such that the LED itself may act as a heat source in maintaining the reaction chamber 106 temperature at or around 60 degrees C during amplification.

### High-Speed Cartridge ID Mode

FIG. 24A shows an alternative embodiment of the cartridge ID timing sequence. In some embodiments, a cartridge ID timing sequence utilizes 4 excitation LEDs instead of 8. This provides an advantage of being able to scan (photodetector multiplexing) at a faster rate (high-speed). This may be advantageous when a user inserts the cartridge 12 into the reader 14 very quickly. The high-speed mode can be used in static or dynamic ID detection.

### C. Sample Collection

In some embodiments, the present disclosure provides one or more parts to collect a sample (i.e., a sample collection, for example, vessel or vial 18). In some embodiments, a sample collection vessel 18 is used to collect and/or contain a sample. In some embodiments, a sample collection vessel 18 comprises one or more target nucleic acid(s). In some embodiments, a sample collection vessel 18 comprises one or more parts that interfaces with a cartridge 12. In some embodiments, a sample collection vessel 18 is sample-specific. In some embodiments, a sample collection vessel 18 is assay-specific.

In some embodiments, a sample collection vessel 18 comprises a sterile swab 16 and a buffer/reagent container.

### D. Detection System

In some embodiments, the present disclosure provides a detection system 10. An exemplary detection system is shown in FIG. 1. The system 10 is designed for use by non-trained users (consumers) and incorporates simple and familiar tasks whenever possible. The detection system 10 is designed to work in a home setting. In some embodiments, the detection system uses only standard wall power (e.g., 110 V AC, using a commercially-available USB charger). The detection system 10 architecture (such as cartridge 12, reader 14, and assay reagents) provides a flexible and expandable system such that new applications and measurement menus/functionality can be added and developed. In some embodiments, the system 10 (namely reader 14) draws from about 100 mA to about 1000 mA of current when powered by the USB charger. In some embodiments, the system include a lithium-ion battery (e.g., rechargeable battery, non-rechargeable battery) to power the device so that it can be made portable, and so that it can be used when AC power is not available (e.g., in locations without power access, during power outages, etc.).

### E. Detection of Nucleic Acid

In some embodiments, systems, methods, compositions, or devices provided herein detect one or more target nucleic acid(s). In some embodiments, a target nucleic acid is a deoxyribonucleic acid (DNA). In some embodiments, a target nucleic acid is a ribonucleic acid (RNA). In some embodiments, a target nucleic acid is single stranded. In some embodiments, a target nucleic acid is double stranded.

In some embodiments, a target nucleic acid is present in a sample. In some embodiments, a sample comprises one or more target nucleic acid(s). In some embodiments, a sample comprises one or more target nucleic acid(s) and nucleic acids other than the one or more target nucleic acid(s). In some embodiments, a target nucleic acid is from a eukaryote. In some embodiments, a target nucleic acid is from a prokaryote. In some embodiments, a target nucleic acid is parasitic (e.g., protozoan), bacterial, viral, or fungal. In some embodiments, a target nucleic acid is from a human.

In some embodiments a sample is an environmental sample. In some embodiments, a sample is a biological sample. In some embodiments, a biological sample is a sample obtained or derived from a biological source (e.g., a tissue or organism or cell culture) of interest. In some embodiments, a source of interest is or comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or bronchoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc.

In some embodiments, cells in a sample are lysed to release nucleic acids. In some embodiments, cells in a sample are lysed within a composition or device as provided herein. In some embodiments, cells in a sample are lysed by heating the sample. In some embodiments, cells in a sample are lysed by heating the sample to about 80° C., 85° C., 90° C., or 95° C.

In some embodiments, systems, methods, compositions, or devices provided herein comprise amplification of a target nucleic acid. One of skill in the art will recognize various methods know in the art to amplify nucleic acids. In some embodiments, systems, methods, compositions, or devices provided herein comprise isothermal amplification of nucleic acid. In some embodiments, isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, polymerase chain reaction (PCR), multiple displacement amplification (MDA), rolling circle amplification (RCA), ligase chain reaction (LCR), or ramification amplification method (RAM). In some embodiments, isothermal amplification is LAMP, for example as described in patents U.S. Pat. No. 9,909,168; 7,374,913; 7,851,186; and 7,846,695. In some embodiments, isothermal amplification is condensed LAMP (cLAMP), for example as described in U.S. 63/470,298 and U.S. 63/511,491. In some embodiments, isothermal amplification occurs at about 50° C., 55° C., 60°C., 65° C., or 70° C. In some embodiments, LAMP occurs at about 50° C., 55° C., 60° C., 65° C., or 70° C. In some embodiments, LAMP or cLAMP occurs at ambient temperatures (e.g., room temperature).

One of skill in the art is aware of various technologies useful in detecting one or more target nucleic acid(s). In some embodiments, detection technologies comprise, for example, absorbance, CRISPR/Cas detection (e.g., SHERLOCK), FRET, or bridged ligation (e.g., INSPECTR as described in WO2020037038A1, the entire contents of which is incorporated by reference herein).

Certain CRISPR/Cas enzymes have been identified that have an ability to non-specifically cleave collateral nucleic acid(s) when activated by binding to a target site in a target nucleic acid recognized by the guide RNA with which the CRISPR/Cas enzyme is complexed. Representative examples of Cas12, Cas13, and Cas14 enzymes have been shown to have such collateral cleavage activity. See. for example, Swarts and Jinek, Mol. Cell. 2019 February 7; 73(3): 589-600.e4; Harrington L. B. et al., Science 2018; 362:839-842; Li S. Y. et al. Cell Res. 2018; 28:491-493; Chen J. S. et al., Science 2018; 360:436-439;

Abudayyeh O. O. et al., Science 2016; 353aaf 5573; East-Seletsky A. et al., Nature 2016; 538:270-273; Gootenberg J. S. et al., Science 2017; 356:438-442; Myhrvold C., et al., Science 2018; 360:444-448; and Gootenberg J. S. et al., Science 2018; 360:439-444. Some CRISPR/Cas enzyme collateral cleavage activity digests or cleaves single strand nucleic acids. Some CRISPR/Cas enzyme collateral cleavage activity digests or cleaves double stranded nucleic acids. Some CRISPR/Cas enzyme collateral cleavage activity digests or cleaves RNA. Some CRISPR/Cas enzyme collateral cleavage activity digests or cleaves DNA. Some CRISPR/Cas enzyme collateral cleavage activity digests or cleaves both RNA and DNA. Collateral activity has been harnessed to develop CRISPR/Cas detection (e.g., diagnostic) technologies that achieve detection of nucleic acids containing the relevant target site (e.g., Cas target nucleic acid), or its complement, in biological and/or environmental sample(s). See, for example Gootenberg, J. S. et al., Science 2017, 356 (438-442); WO2019/011022; U.S. Pat. Nos. 10,494,664; 10,337,051; and 10,266,887; sherlock. bio/better-faster-affordable-diagnostic-testing.

SHERLOCK is a detection technology comprising steps of: contacting a CRISPR/Cas complex comprising a Cas protein with collateral cleavage activity, a guide RNA selected or engineered to be complementary to a target nucleic acid (e.g., a Cas target nucleic acid sequence), and a sample potentially comprising a Cas target nucleic acid (see, e.g., WO 2018/107129, WO 2019/011022, which are incorporated herein by reference). In some embodiments, CRISPR/Cas-based detection may be a CRISPR/Cas 13-based detection system. In some embodiments, a CRISPR/Cas-based detection system is a CRISPR/Cas 12-based detection system. In some embodiments, a CRISPR/Cas13-or CRISPR/Cas12-based detection system is a SHERLOCK detection system. One of skill in the art is aware of various CRISPR/Cas enzymes that may be useful in the systems, compositions, and methods as provided herein. For examples CRISPR/Cas enzymes are described in WO2016/166340; WO2016/205711; WO/2016/205749: WO2016/205764; WO2017/070605; WO/2017/189308; WO2021/154866A1; WO2023/009526 the entire contents of each of which is incorporated by reference herein. In some embodiments, SHERLOCK detection technology also comprises a detectably labeled nucleic acid probe. Cleavage of the detectably labeled nucleic acid probe by the collateral cleave activity of a CRISPR/Cas enzyme can induce or increase the detectable label indicating the presence of a target nucleic acid. In some embodiments, a detectably labeled nucleic acid probe is labeled with a fluorescent label. In some embodiments, a fluorescent label comprises a fluorescent group at the 5′ end and a quenching group at the 3′ end. In some embodiments, a fluorescent group is hexachlorofluorescein (HEX) or carboxyfluorosceine (FAM). In some embodiments, a quenching group is a black hole quencher (BHQ).

In some embodiments, detection of one of more nucleic acid(s) comprises obtaining from a subject a biological sample via a sample container (see for example FIGS. 1-5); incubating the biological sample with at least one of a reagent and a buffer via the sample container, thereby producing a biological solution; inserting the sample container into a cartridge such that the biological solution flows into an interior chamber of the cartridge, the interior chamber comprising a first heating zone; inserting the cartridge into an electronic reader comprising multiple heating elements for creating the first heating zone and a second heating zone within the cartridge; performing a lysis step on the biological solution within the first heating zone; passively cooling the biological solution by opening an internal passage of the cartridge such that the biological solution flows via gravity feed into the internal passage, the internal passage being fluidly downstream of, and vertically below, the interior chamber; amplifying one or more target nucleic acid(s) in the biological solution by isothermal amplification within the second heating zone which comprises multiple reaction chambers fluidly downstream of the internal passage; wherein each of the multiple reaction chambers comprises: a CRISPR/Cas enzyme having collateral cleavage activity, a guide RNA that specifically hybridizes with one target nucleic acid, a detectably labeled nucleic acid probe, wherein hybridization of the guide RNA with the target nucleic acid induces or increases collateral cleavage activity of the CRISPR/Cas enzyme and the CRISPR/Cas enzyme cleaves the detectably labelled nucleic acid probe, wherein cleavage of the detectably labeled nucleic acid probe results in an increase in detectable label, illuminating the biological solution within each of the multiple reaction chambers via a plurality of optical energy sources, each energy source of the plurality of optical energy sources being disposed in the vicinity of one of the multiple reaction chambers, and determining the presence of at least one target nucleic acid within the biological solution based on presence or level of the detectable label via a detection device (FIG. 28).

### F. Method and Use

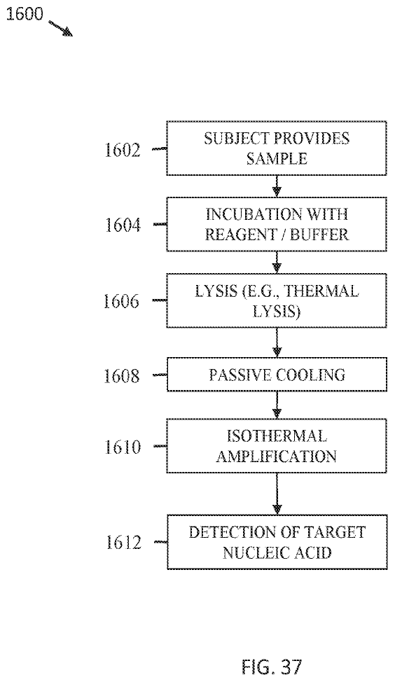

Referring to FIG. 37, in some embodiments, the present disclosure provides a method 1600 for detecting one or more target nucleic acid(s) using a reader 14, a cartridge 12, a sample collection, or a combination of those. In some embodiments, methods according to the present disclosure comprise one or more of the following steps:
i. collecting a sample;
ii. thermal lysis of cells in the sample;
iii. passive sample cooling;
iv. transfer to one or more reaction chambers;
V. isothermal amplification;
vi. Cas enzyme activation; and
vii. detection.
   For example, as shown in FIG. 37 and as described herein, at step 1602, the method 1600 may include providing a biological sample (for example, saliva, mucus, etc.) by the subject. At step 1604, the method 1600 may include incubation of the sample with reagent/buffer. At step 1606, the method 1600 may include thermal lysis of cells in the sample-reagent mixture. At step 1608, the method 1600 may include passive cooling of the sample-reagent mixture following lysis (for example, while the sample travels (i.e., flows) between the lysis chamber 94 and the reaction chambers 106). At step 1610, the method 1600 may include isothermal amplification in the reaction chambers 106. At step 1612, the method 1600 may include detection of the target nucleic acid.

The total workflow for the test is approximately 20 to 40 minutes in length, depending on the assay being run. The user steps to begin the test run are less than 2 minutes, and the remainder of the test is run automatically. FIG. 3 shows a combined summary of the user workflow and assay workflow.

In some embodiments, a sample is collected. In some embodiments, a user collects a sample from themselves using a standard sterile swab 16. Alternatively, an adult may collect a nasal swab sample from a child or other subject.

In some embodiments, a sample is eluted into a buffer. In some embodiments, a user opens a buffer tube and swirls a swab head to elute the sample into a buffer. Alternatively, a standard extraction tube can be used for the elution.

In some embodiments, a sample is transferred to a cartridge. In some embodiments, a buffer containing a sample is provided via a sample inlet port. Exemplary transfer is shown in FIG. 5. In some embodiments, a cap is used to seal the cartridge. Exemplary cap is shown in FIG. 5. After a sample is added to a cartridge, the cartridge is inserted into a reader.

In some embodiments, a reader automatically provides fluidic control, thermal control, and optical measurement of the cartridge 12 within a 15-to-45-minute (e.g., 20 to 40 minutes) test time. Upon completion of the test, a result is displayed utilizing easy to read LEDs (e.g., LED illuminates for positive result). In some embodiments, a cartridge 12 contains a label indicating the test type and the assay and/or analytical result.

In some embodiments, a cartridge 12 is inserted in a reader 14. In some embodiments, insertion of a cartridge 12 into the reader 14 initiates a detection method. Exemplary cartridge 12 installation is shown in FIG. 6.

In some embodiments, a reader 14 automatically detects the presence of a cartridge 12. In some embodiments, a reader 14 automatically detects the type of cartridge 12.

In some embodiments, a reader 14 communicates its status (e.g., Power ON/OFF, Ready/Running/Complete, Invalid Result or Error) by LEDs. In some embodiments, LEDs are positioned adjacent to corresponding labels 290. In some embodiments, the label 290 is on the case (i.e., the outer surface of the reader 14). An example of a status LED scheme is show in FIG. 7.

In some embodiments, temperature is configurable via software. In some embodiments, step time is configurable via software. In some embodiments, a run sequence comprises a heating cycle and an amplification cycle.

### Lysis of Cells in Sample

In some embodiments, a method according to the present invention comprises a step of heating a sample. In some embodiments, a sample is heated to lyse cells in the sample. In some embodiments, a heating cycle leads to cell lysis in the sample. In some embodiments, a sample is heated to at least 80° C., such as at least 85° C., such as 90° C. In some embodiments, a sample is heated in a lysis chamber 94 in the cartridge 12.

In some embodiments, a lysis chamber 94 area of the cartridge (FIG. 8) is heated (e.g., to 90° C.) by the reader 14 for the duration set by the assay sequence. In some embodiments, a lysis chamber 94 area is then cooled passively to below 60° C. before the next step.

In some embodiments, a sample is transferred from the lysis chamber 94 to the reaction chambers 106. In some embodiments, a sample is being split into individual reaction chambers 106. In some embodiments, a sample is transferred to one or more reaction chambers 106. In some embodiments, a sample is transferred to two or more reaction chambers 106. In some embodiments, a sample is transferred to three or more reaction chambers 106. In some embodiments, a sample is transferred to four or more reaction chambers 106. In some embodiments, a sample is transferred to five or more reaction chambers 106. In some embodiments, a sample is transferred to six or more reaction chambers 106. In some embodiments, a sample is transferred to seven or more reaction chambers 106. In some embodiments, a sample is transferred to eight or more reaction chambers 106. In some embodiments, a reaction chamber 106 can comprise a volume in the range of about 10 μl to about 100 μl, such as in the range of 15 μl to about 80μl, such as in the range of 20 μl to about 60 μl, such as in the range of 30 μl to about 50 μl, such as a 40 μl sample. In some embodiments, a sample transfer is driven by gravity flow (e.g., as a reader 14 and a cartridge 12 are positioned in a vertical orientation). Exemplary vertical orientation is shown in FIG. 9.

In some embodiments, a detection system 10 uses rehydration of a lyophilized bead within each reaction chamber 106 to verify the filling of the reaction chamber. Filling of the chamber can be detected by, e.g., a change in fluorescence that occurs when a lyophilized bead 268 is rehydrated.

### Amplification Cycle

In some embodiments, an amplification cycle is performed in the temperature range of about 50° C. to about 70° C., such as about 55° C. to about 65° C. In some embodiments, amplification is performed at isothermal conditions.

In some embodiments, a reader 14 uses a film heater to heat the reaction chamber area 126 of the cartridge 12 to about 50° C. to about 70° C., such as about 55° C. to about 65° C., such as to about 60° C. for the duration of the amplification.

In some embodiments, amplification is LAMP amplification.

### Detection

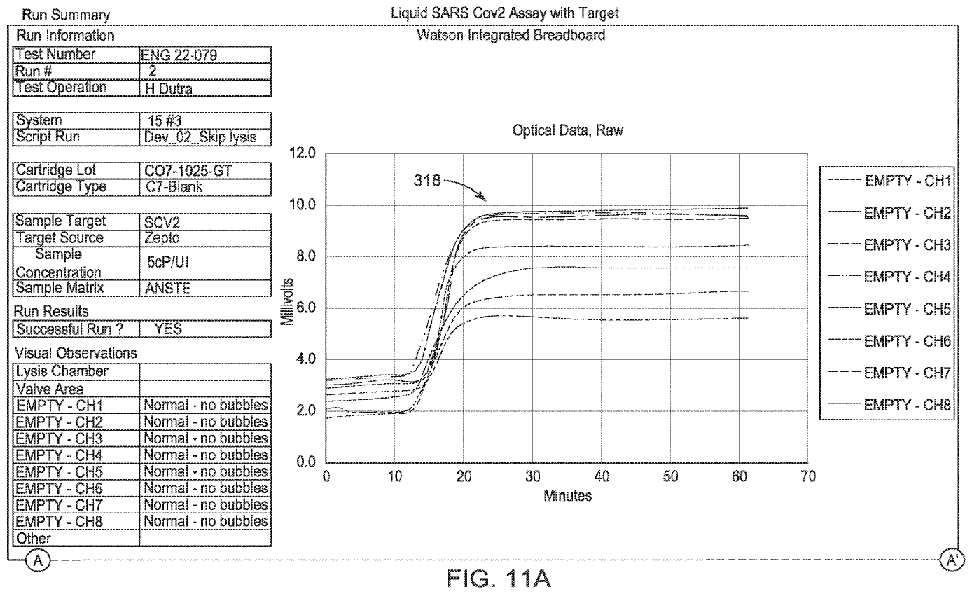

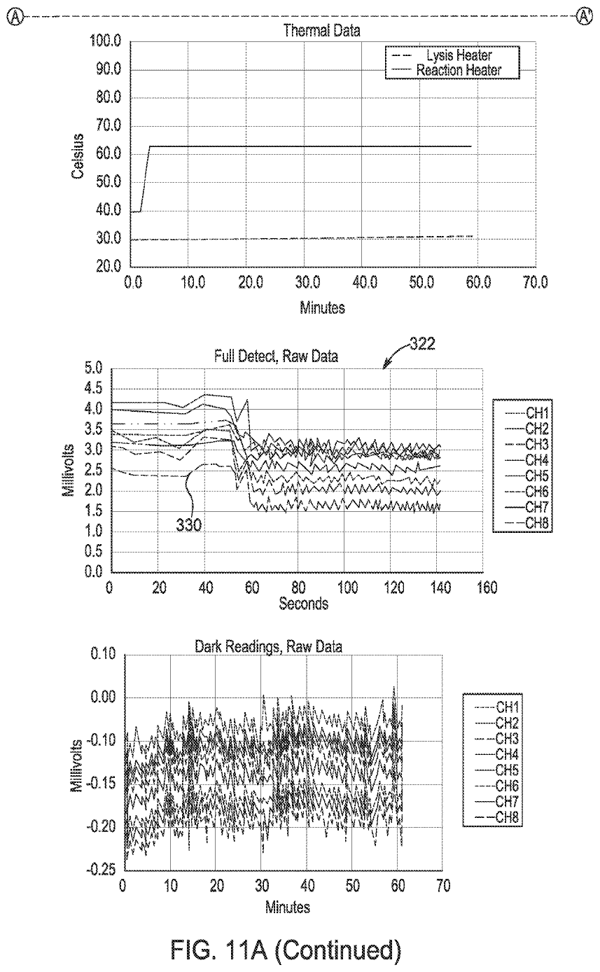

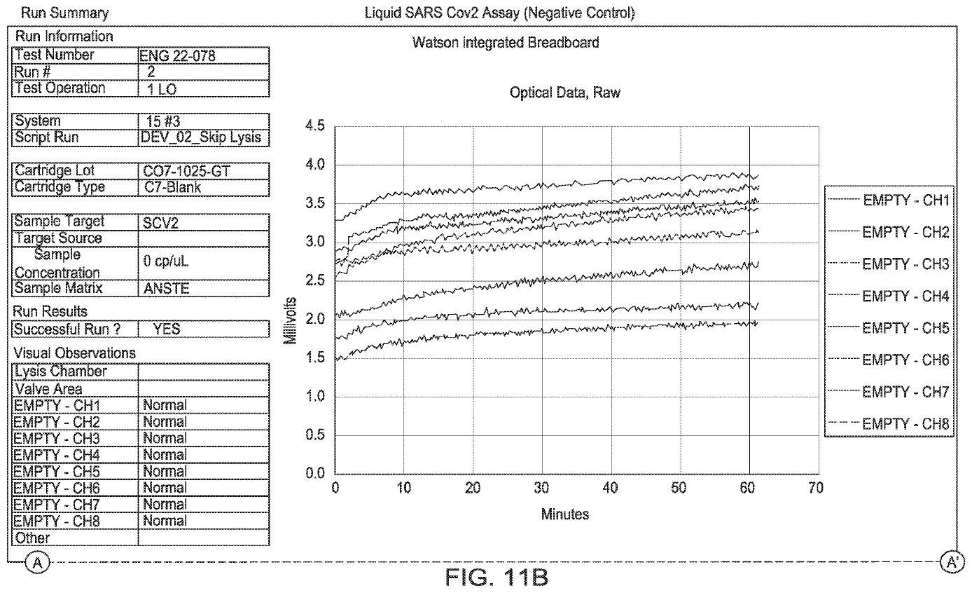

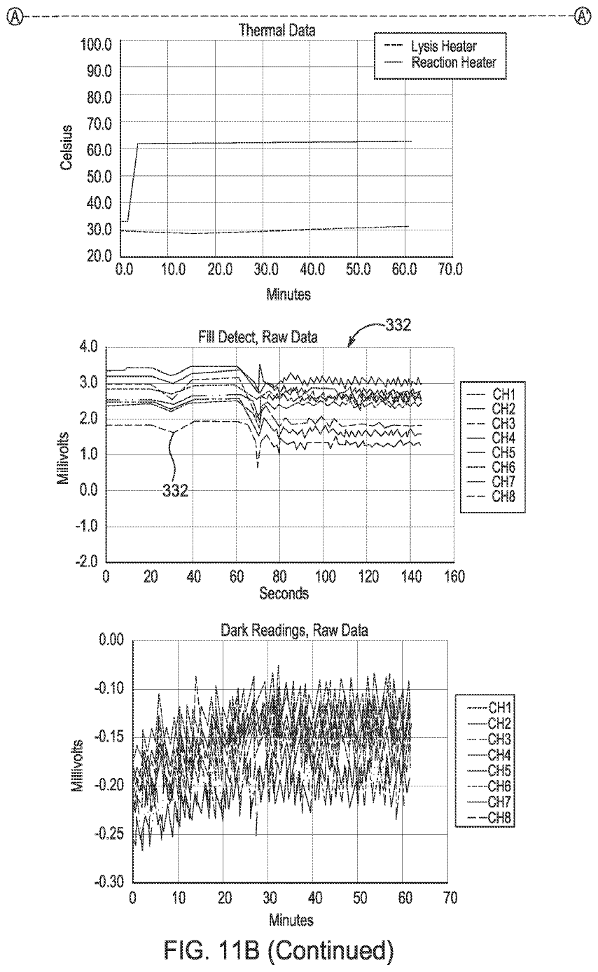

In some embodiments, an amplification product is detected. In some embodiments, a LAMP amplification product is detected. In some embodiments, an operating software detects whether amplification has occurred for each reaction chamber. In some embodiments, detection is based on raw optical data. FIG. 11 shows two examples of raw optical data plotted on a graph. The Liquid SARS-CoV-2 samples (FIG. 11A) show amplification 318 in about 15 minutes, and the negative control samples (FIG. 11B) show no amplification after 60 minutes.

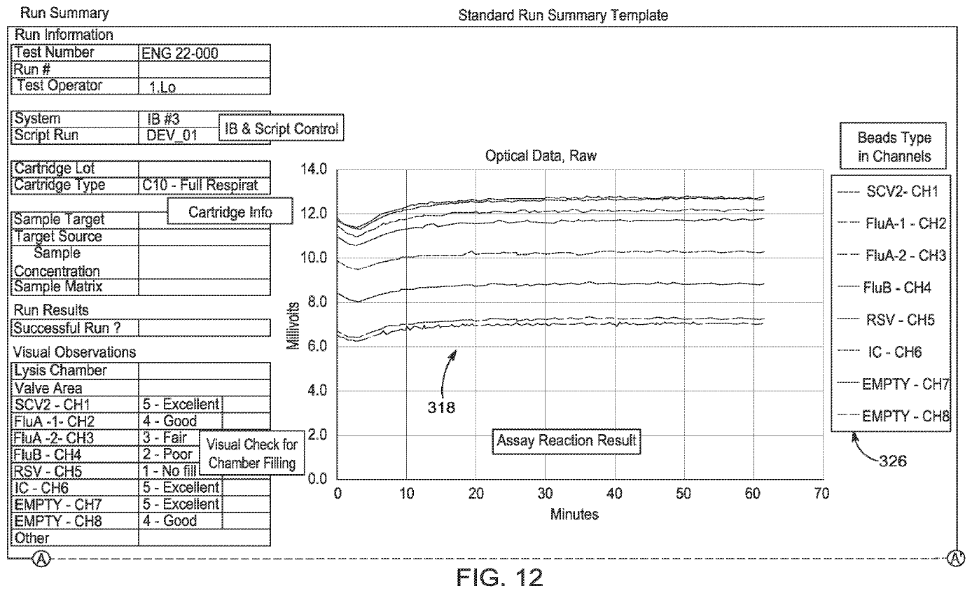

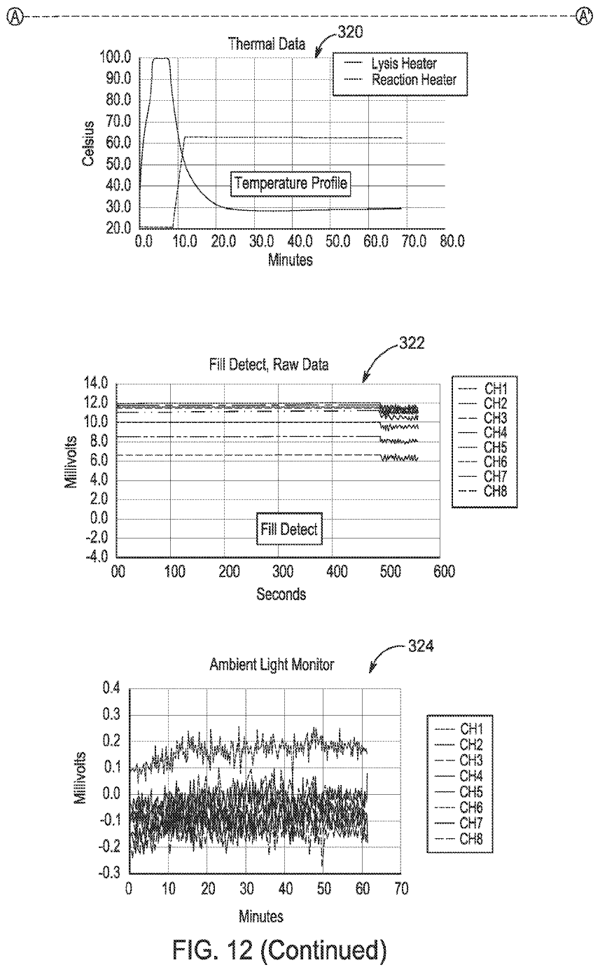

In some embodiments, raw data is plotted for analysis in a standardized format that also captures key metadata from the run, such as the identification of the instrument (reader 14), sample type, and cartridge 12 type (see FIG. 12). The displayed data may include assay reaction results 318 for each of the 8 channels, temperature profiles 320 for each of the two heating zones, fill detect signals 322 for each of the eight channels, and ambient light monitoring 324 for each of the eight channels.

As shown in FIG. 11A, the fill detect signals 322 may include a drop 330 in amplitude when fluid enters the reaction chambers 106 due to less light from the LEDs reaching the photodetectors 144, 162 when the reaction chambers 106 are filled with fluid. In addition, as shown in FIG. 11B, the fill detect signals 322 may experience an increase 332 in magnitude when the cartridge is inserted due to increased reflectance. The fill detect algorithm 230, as shown in FIG. 24B, verifies that the drop 330 in magnitude is observed in each (i.e., all 8) of the reaction chambers 106, thereby confirming that all of the reaction chambers 106 are filled with fluid. As shown in the legend 326 in FIG. 12, each of the channels (i.e., reaction chambers 106) may hold or contain a lyophilized bead corresponding to a different indication (for example, including but not limited to: respiratory viruses such as SARS-CoV-2 (SCV2), FluA-1, FluA-2, FluB, RSV (respiratory syncytial virus), as well as sexually transmitted infections (STIs) including Chlamydia trachomatis (CT), Neisseria gonorrhoeae (NG), and Trichomonas vaginalis (TV), as well as other potential indications, infections, bacteria, and/or viruses. That is, each lyophilized bead may contain reagents, primers for nucleic acids, enzymes, etc., that are specifically chosen for performing a particular assay to detect a particular indication. This arrangement allows multiple assays corresponding to multiple viruses or conditions to be performed simultaneously based on a single patient sample, by having a different lyophilized bead composition in each reaction chamber 106.

In some embodiments, a reader 14 displays the assay results. In some embodiments, a reader 14 displays the assay results via illuminated LEDs. In some embodiments, illuminated LEDs correspond to labels 290 on a cartridge 12. That is, each cartridge 12 may have different printed labels 290 corresponding to the conditions being tested in the cartridge 12, so that the labels are visible adjacent to LEDs on the reader 14 so that the same LEDs on the reader 14 may indicate a variety of different conditions depending on what cartridge 12 is inserted. In some embodiments, a target nucleotide acid is detected in a reaction chamber 106 resulting in a positive result. In some embodiments, an assay that is considered positive will be indicated. Exemplary indication is shown in FIG. 2. If no assays were positive and the test run was valid (including control reactions), the LED corresponding to ‘NEG’ (i.e., indicating that all assays are negative) will illuminate, as shown in FIG. 2B.

### EXEMPLIFICATION

### Example 1

The present Example demonstrates detection of SARS-CoV-2 virus using a detection system according to the present invention. FIG. 11 shows two examples of raw optical data plotted on a graph. The vertical axis shows optical signal at a photodetector in millivolts, while the horizontal axis shows reaction time in minutes. Liquid samples containing SARS-CoV-2 (FIG. 11A) show amplification in about 15 minutes (i.e., an increase in fluorescence), and the negative control samples (FIG. 11B) show no amplification after 60 minutes (i.e., no increase in fluorescence). The eight channels of optical signals in FIG. 11A and FIG. 11B correspond to signals measured by eight separate photodetectors, each corresponding to a different reaction chamber in a cartridge containing eight reaction chambers. While the example shown here had the same detection assay (i.e., SARS-CoV-2) in each of the eight reaction chambers, in other examples each reaction chamber may feature a different assay to detect a different target nucleic acid.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A system for detecting the presence of a nucleic acid associated with at least one indication in a biological solution, the system comprising:
an electronic reader for performing one or more assays and displaying the results thereof;
a cartridge configured to be inserted into the reader, the cartridge comprising a cartridge assembly; and
a sample collection vessel for collecting a biological sample and transferring it into an interior chamber of the cartridge.

2. The system of claim 1, the system further comprising an internal assembly comprising the cartridge assembly once the cartridge is inserted into the reader, the internal assembly further comprising:
a heating unit comprising one or more heating elements; and
an optical assembly comprising at least one LED and at least one photodetector.

3. The system of claim 2, wherein the cartridge assembly comprises a lysis chamber for receiving the biological sample from the sample collection vessel, and one or more reaction chambers disposed fluidly downstream from the lysis chamber.

4. The system of claim 3, wherein the one or more heating elements comprise a first heating element for maintaining the lysis chamber at a first temperature and a second heating element for maintaining the one or more reaction chambers at a second temperature.

5. The system of claim 3, wherein the cartridge assembly comprises:
at least one of a buffer and a reagent disposed within the lysis chamber;
at least one lyophilized lysis bead disposed within the lysis chamber; and
a lyophilized PCR bead disposed within each one of the one or more reaction chambers.

6. (canceled)

7. The system of claim 3, wherein the cartridge assembly comprises a polymer casing forming a back surface of the cartridge and a film layer comprising a front surface of the cartridge, the polymer casing and film layer sandwiching each of the buffer, reagent, at least one lyophilized lysis bead and/or lyophilized PCR bead therebetween.

8. (canceled)

9. The system of claim 1, wherein the at least one LED comprises a first LED in optical communication with a reaction chamber of the one or more reaction chambers,
wherein the at least one LED is configured to illuminate an interior of the one or more reaction chambers, and
wherein the at least one photodetector is configured to measure fluorescence emitted from an illuminated interior of the one or more reaction chambers.

10-11. (canceled)

12. The system of claim 1, wherein each of the at least one LED and the at least one photodetector is integrated into a printed circuit board assembly (PCBA), and
wherein the heating unit is integrated into the PCBA,

13-14. (canceled)

15. The system of claim 5, wherein the one or more reaction chambers comprise multiple reaction chambers, and
wherein a type of the lyophilized PCR bead in a first reaction chamber of the multiple reaction chambers is different from a type of lyophilized PCR bead in a second reaction chamber of the multiple reaction chambers.

16-17. (canceled)

18. The system of claim 3, wherein light emitted from the at least one LED excites at least one nucleic acid contained within the one or more reaction chambers without passing through an optical lens.

19. The system of claim 2, further comprising a mechanical assembly, wherein the mechanical assembly is configured to laterally move the cartridge within the reader after the cartridge is inserted into the reader, upon the closing of a lid of the reader, the mechanical assembly comprising:
at least one linkage coupling the lid to an internal apparatus disposed within the reader; and
a cam coupled to both the at least one linkage and the internal apparatus, the cam converting the closing movement of the lid to lateral movement of the internal apparatus.

20. The system of claim 1, wherein the cartridge comprises at least one identification mark configured to be illuminated by the at least one LED, and
wherein the at least one identification mark comprises at least one printed barcode.

21-24. (canceled)

25. A method of identifying an attribute of a cartridge configured to be inserted into an electronic reader, the method comprising:
providing the electronic reader, the electronic reader comprising at least one internal LED, at least one internal photodetector, and at least one internal microprocessor communicatively coupled to both the at least one photodetector and the at least one LED;
providing the cartridge, the cartridge comprising at least one identification mark;
inserting the cartridge into the reader;
illuminating, by the at least one internal LED, the at least one identification mark, thereby creating an illuminated identification mark;
sensing, by the at least one internal photodetector, an optical signature of the illuminated identification mark; and
identifying, by the at least one internal microprocessor, at least one attribute of the cartridge based on the illuminated identification mark.

26. The method of claim 25, wherein illuminating the at least one identification mark occurs as the cartridge is inserted into the electronic reader, and wherein the at least one identification mark comprises at least one barcode.

27. (canceled)

28. The method of claim 26, wherein the at least one internal LED comprises at least a first LED and a second LED,
wherein the at least one barcode comprises:
a first barcode used to quantify the speed at which the cartridge is inserted into the electronic reader, the first barcode being illuminated by the first LED; and
a second barcode used to identify the at least one attribute of the cartridge, the second barcode being illuminated by the second LED, and
wherein the at least one microprocessor uses the quantified speed to calibrate the optical signature of the second barcode, thereby allowing the attribute of the cartridge to be identified.

29-30. (canceled)

31. The method of claim 25, wherein the at least one internal photodetector comprises a dual mode photodetector configured to measure light emitted from at least one LED within both a first spectrum and a second spectrum, and
wherein the second spectrum does not overlap with the first spectrum.

32. The method of claim 31, wherein the first spectrum comprises wavelengths in a range from about 600 nm to about 660 nm, and
wherein the second spectrum comprises wavelengths in a range from about 730 nm to about 900 nm.

33. The method of claim 25, wherein the at least one identification mark comprises one or more printed marks on a label disposed on a surface of the cartridge.

34. The method of claim 33, wherein the at least one LED comprises from about 4 to about 12 identification LEDs,
wherein the one or more printed marks comprises from about 4 to about 12 printed marks, each printed mark being positioned to correspond to a position of one of the identification LEDs such that presence or lack of presence of a printed mark at a position may be sensed by the identification LED at the corresponding position.

35-36. (canceled)

37. The method of claim 25, wherein the attribute comprises a type of one or more assays that may be performed using the cartridge.

38. (canceled)

39. The method of claim 33, wherein the one or more printed marks comprises one or more position sensing features.

40. The method of claim 39, wherein the one or more position sensing features comprises:
a first position sensing feature positioned on the label such that a centerline of the first position sensing feature is positioned so as to be slightly above a centerline of a first component of the reader when the cartridge is positioned correctly within the reader; and
a second position sensing feature positioned on the label such that a centerline of the second position sensing feature is positioned so as to be slightly below a centerline of a second component of the reader when the cartridge is positioned correctly within the reader;
the method comprising determining, by the microprocessor, that the cartridge is or is not positioned correctly within the reader based on:
1) the position of the first position sensing feature relative to the first component of the reader, and
2) the position of the second position sensing feature relative to the second component of the reader,
wherein each of the first component and the second component comprises at least one of the at least one internal photodetector and the at least one internal LED.

41. The method of claim 25, wherein the reader is configured to identify the attribute of the cartridge based on multiple modes of operation, the multiple modes of operation comprising:
a static identification mode comprising identification of the attribute, via the at least one identification mark and the reader, after the cartridge is inserted into the reader; and
a dynamic identification mode comprising identification of the attribute, via the at least one identification mark and the reader, while the cartridge is being inserted into the reader.

42-96. (canceled)
